SEQUENCE LISTING (18 sequences)

SEQ ID NO: 1 - DNA, 20 bp, Artificial Sequence
  Synthetic sequence
ggctctcatggaatggctaa

SEQ ID NO: 2 - DNA, 20 bp, Artificial Sequence
  Synthetic sequence
tctgcgaaacaggttttacg

SEQ ID NO: 3 - DNA, 100 bp, Artificial Sequence
  Genomic Influenza A nucleotide sequence
cagatcttgaggctctcatggaatggctaaagacaagagaattttaggatttgtgttcacactgcagcgtagacgctttgtccaaaatgccctaaatggg

SEQ ID NO: 4 - DNA, 96 bp, Artificial Sequence
  Synthetic sequence
cagatcttgaggctctcatggaatggctaaagacaagagaattttagggtgttcacactgcagcgtagacgctttgtccaaaatgccctaaatggg

SEQ ID NO: 5 - DNA, 83 bp, Artificial Sequence
  Synthetic sequence
tctaagcagcccaaccgaagggaagacagatgccagcactaatgatacggcgaccaccgactcatggaatggctaaagacaag

SEQ ID NO: 6 - DNA, 21 bp, Artificial Sequence
  Synthetic sequence
tagactctgccttggaatgga

SEQ ID NO: 7 - DNA, 22 bp, Artificial Sequence
  Synthetic sequence
ttcttctaagtagtgtctcggg

SEQ ID NO: 8 - DNA, 100 bp, Artificial Sequence
  Genomic Influenza B nucleotide sequence
gaatttgacctagactctgccttggaatggataaaaagaaagcactaattggtgcctctatctagaaaaagaagattcatcacagagcccttatcgggaa

SEQ ID NO: 9 - DNA, 96 bp, Artificial Sequence
  Synthetic sequence
gaatttgacctagactctgccttggaatggataaaaagaaagcactaatgcctctatctagaaaaagaagattcatcacagagcccttatcgggaa

SEQ ID NO: 10 - DNA, 81 bp, Artificial Sequence
  Synthetic sequence
tctaagcagcccaaccgaagggaagacagatgccagcactaatgatacggcgaccaccgaactctgccttggaatggataa

SEQ ID NO: 11 - DNA, 21 bp, Artificial Sequence
  Synthetic sequence
agacggcatcatatgggttgc

SEQ ID NO: 12 - DNA, 23 bp, Artificial Sequence
  Synthetic sequence
gaaggagttccttgttgtaacgg

SEQ ID NO: 13 - DNA, 103 bp, Artificial Sequence
  Genomic SARS-CoV-2 nucleotide sequence
gtgctaacaaaagacggcatcatatgggttgcaactgaggatcacattggcacccgcaatcgtgctacaacttcctcaaggaacaacattgccaaaaggcttc

SEQ ID NO: 14 - DNA, 99 bp, Artificial Sequence
  Synthetic sequence
gtgctaacaaaagacggcatcatatgggttgcaactgaggatcacattcccgcaatcgtgctacaacttcctcaaggaacaacattgccaaaaggcttc

SEQ ID NO: 15 - DNA, 83 bp, Artificial Sequence
  Synthetic sequence
tctaagcagcccaaccgaagggaagacagatgccagcactaatgatacggcgaccaccgacatatgggttgcaactgagggag

SEQ ID NO: 16 - DNA, 22 bp, Artificial Sequence
  Synthetic sequence
aagacggcatcatatgggttgc

SEQ ID NO: 17 - DNA, 23 bp, Artificial Sequence
  Synthetic sequence
ggcaatgttgttccttgaggaag

SEQ ID NO: 18 - DNA, 72 bp, Artificial Sequence
  Synthetic sequence
ccgtaacgtggcactggaccactactaggcgttacagcttcaacacctggaagacggcatcatatgggttgc

(19) United States
(12) Patent Application Publication
(10) Pub. No.: US 2021/0292829 A1
(43) Pub. Date: Sep. 23, 2021

(54) HIGH THROUGHPUT ASSAYS FOR DETECTING INFECTIOUS DISEASES USING CAPILLARY ELECTROPHORESIS

(71) Applicant: BillionToOne, Inc., Menlo Park, CA (US)

(72) Inventors: Devon Brian Chandler Brown, Campbell, CA (US); Anna Bueno, Santa Clara, CA (US); David Tsao, San Carlos, CA (US); Oguzhan Atay, Menlo Park, CA (US)

(21) Appl. No.: 17/210,488

(22) Filed: Mar. 23, 2021

Related U.S. Application Data
(60) Provisional application No. 62/993,556, filed on Mar. 23, 2020.
(60) Provisional application No. 63/006,507, filed on Apr. 7, 2020.

(51) Int. Cl. C12Q 1/6858; C12Q 1/6853; C12Q 1/686

(52) U.S. Cl. CPC C12Q 1/6858; C12Q 1/686; C12Q 1/6853

(57) ABSTRACT

Aspects of the present disclosure include methods of detecting the presence or absence of one or more infectious diseases using quantitative approaches. In some aspects, the methods of the present disclosure include generating a spike-in mixture including target sample molecules (e.g., endogenous sample) and artificial molecules (e.g., spike in molecule, synthetic target-associated molecule), amplifying the spike in mixture to generate a co-amplified spike in mixture, and performing capillary electrophoresis to detect the presence or absence of one or more infectious diseases.

### 1. CROSS-REFERENCE TO RELATED APPLICATIONS

This application claims the benefit of U.S. Provisional Application No. 62/993,556, filed Mar. 23, 2020, and U.S. Provisional Application No. 63/006,507, filed Apr. 7, 2020, which are hereby incorporated in their entirety by reference.

### 2. SEQUENCE LISTING

The instant application contains a Sequence Listing which has been submitted via EFS-Web and is hereby incorporated by reference in its entirety. Said ASCII copy, created on Mar. 23, 2021, is named 48411SEQ LISTINGST25, and is 5 kilobytes in size.

### 3. SUMMARY

Aspects of the present disclosure include methods of detecting the presence or absence of one or more infectious diseases using quantitative approaches. In some aspects, the methods of the present disclosure include generating a spike-in mixture including target sample molecules (e.g., endogenous sample) and artificial molecules (e.g., spike in molecule, synthetic target-associated molecule), amplifying the spike in mixture to generate a co-amplified spike in mixture, and performing capillary electrophoresis to detect the presence or absence of one or more infectious diseases. Aspects of the present disclosure include systems for carrying out the methods of the present disclosure.

In some aspects, capillary electrophoresis involves encoding molecular information in a sequencing output (e.g., sanger sequencing) and decoding the sequencing information to generate quantitative signals for determining the presence or absence of one or more infectious diseases. By adding a spike-in artificial RNA or DNA sample with a sequence very similar to the endogenous sample's sequence (e.g. infectious disease), but offset by a set number of bases or a variable intra-sequence region, Sanger sequencing will generate an output with mixed sequence traces composed of the combination of the spike-in artificial sequence and endogenous sequences when the sample is positive, and only spike-in artificial sequence trace when the sample is negative for the infectious disease. This allows a perfect intrasample control for extraction, RT, and PCR reactions. At the same time, extracting the sequence information gives qSanger a very high specificity for all positive results, while also allowing population-level analyses such as mutation clustering and help with contact tracing. In addition, the ratio of the amplitudes of corresponding bases between the endogenous and spike-in artificial sequences at offset positions reflects the ratio of the molecular abundances of the two sequences. The combining the amplitude ratios of multiple corresponding bases computationally can be used to estimate the viral load over 1000-fold dynamic range using a single capillary

In other aspects of the present methods, capillary electrophoresis involves a fragment analysis approach for detecting the presence or absence of one or more infectious diseases. Fragment analysis is similar to Sanger in that it is run via Capillary Electrophoresis (CE) using the same DNA Analyzer instrument. The use of CE results in a measurable size separation of signals, making a qSanger-like analysis is possible. Rather than labeling each base as in Sanger, fragment analysis uses fluorescent end-point labeling wherein fluorescent dyes are attached to labeling primers and incorporated into samples through a PCR reaction. Fragment analysis allows for target molecules to be separated by both size and color space, and thus a single injection can generate data for many independent loci. Additionally, fragment analysis requires only two PCR reactions (amplification and labeling) and does not involve any bead purification as labeled product is directly diluted and denatured in formamide for injection.

In one aspect, the present disclosure provides a method of detecting the presence or absence of a coronavirus in a sample obtained from a subject. In some embodiments, the method comprises generating a spike-in mixture including sample molecules from the sample and synthetic target-associated molecules, wherein the synthetic target-associated molecules comprise: a target-matching region having a nucleotide sequence that matches a corresponding nucleotide sequence in a first region of the coronavirus's nucleotide sequence; and a target-variation region that is distinguishable from a second region of the coronavirus's nucleotide sequence, the target-variation region having a nucleotide sequence with an insertion or deletion as compared to a corresponding nucleotide sequence in the second region of the coronavirus's nucleotide sequence; co-amplifying the spike-in mixture to generate a co-amplified spike-in mixture; performing capillary electrophoresis on the co-amplified spike-in mixture to generate a chromatogram-related output comprising a plurality of chromatogram intensities, the intensities including one or more peaks. In some embodiments, the one or more peaks include at least one of: a peak associated with the synthetic target-associated molecules; or a peak associated with the coronavirus nucleotide sequence. The method further includes determining the presence or absence of the coronavirus based on the peaks, wherein a position of the peak associated with the synthetic target-associated molecules is offset as compared to an expected location of the peak associated with the coronavirus nucleotide sequence.

In some embodiments, the generating a spike-in mixture including sample molecules from the sample and synthetic target-associated molecules comprises: mixing the target-associated molecules with the sample molecules; and performing reverse transcription on the spike-in mixture to convert the sample molecules into DNA.

In some embodiments, the method does not include RNA extraction of the sample molecules.

In some embodiments, the chromatogram-related output comprises alignment positions corresponding to the chromatogram intensities, wherein the chromatogram intensities comprise first peaks associated with: the target-matching region of the synthetic target-associated molecules; the target-variation region of the synthetic target-associated molecules; and the region of the sample molecules of the subject that corresponds to the target-variation region of the synthetic target-associated molecules. In some embodiments, for each of the different pairs, the base of the nucleotide sequence of the synthetic target-associated molecule corresponds to a first alignment position that is different from a second alignment position corresponding to the base of the nucleotide sequence of the sample molecule, and wherein the alignment positions of the chromatogram-related output comprise the first and the second alignment positions.

In some embodiments, co-amplifying the spike-in mixture comprises amplifying the synthetic-target associated molecules and the sample molecules with a set of primers, wherein the set of primers include nucleotide sequences that are complementary or reverse complementary to the target matching region of the synthetic target-associated molecules and are complementary or reverse complementary to the first region of the coronavirus's nucleotide sequence.

In some embodiments, amplifying is performed using polymerase chain reaction (PCR).

In some embodiments, the set of primers further comprise universal-tailed primers comprising universal tailed sequences. In some embodiments, the set of primers comprise forward and reverse primers. In some embodiments, the forward and reverse primers further comprise one or more fluorescently labeled tags. In some embodiments, the fluorescently labeled tags are attached at the 5′ end of the primer sequences.

In some embodiments, the co-amplified mixture comprises synthetic target-associated amplicon products and, when coronavirus is present in the sample, coronavirus amplicon products, the synthetic target-associated amplicon products comprising a nucleotide length that is shorter than a nucleotide length of the second region of the coronavirus's nucleotide sequence.

In some embodiments, the nucleotide length of the synthetic target-associated amplicon products is shorter by 1-50 nucleotides.

In some embodiments, the co-amplified mixture comprises synthetic target-associated amplicon products and, when coronavirus is present in the sample, coronavirus amplicon products, the target-associated amplicon products comprising a nucleotide length that is longer than the nucleotide length of the second region of the coronavirus's nucleotide sequence. In some embodiments, the nucleotide length of the synthetic target-associated amplicon products is longer by 1-50 nucleotides.

In some embodiments, each chromatogram peak comprises one or more peak intensities associated with at least one of: the target-matching region of the synthetic target associated molecules; the target variation region of the synthetic target associated molecules; or the region of the coronavirus's nucleotide sequence that corresponds to the target-variation region of the synthetic target-associated molecules.

In some embodiments, the peak intensity of the region of the sample molecules that corresponds to the target-variation region of the synthetic target-associated molecules includes a peak intensity position that is offset as compared to a peak intensity position of the target-variation region, wherein the offset corresponds to the insertion or deletion of one or more nucleotides in the target-variation region. In some embodiments, the peak intensity of the region of the sample molecules that corresponds to the target-variation region of the synthetic target-associated molecules includes a peak intensity position that is offset as compared to the peak intensity position of the target-variation region, wherein the peak intensity position is offset by a distance away from the peak intensity of the synthetic target-associated molecule.

In some embodiments, the method further comprises determining an absolute abundance of coronavirus nucleotide molecules by comparing the peak intensities of the region of the coronavirus's nucleotide sequence that corresponds to the target-variation region of the synthetic target-associated molecules to the peak intensities of the target-variation region of the synthetic target-associated molecules, wherein the absolute abundance is determined based on a known number of synthetic target-associated molecules added to the sample spike-in mixture.

In some embodiments, the method further comprises calculating the ratio of peak intensities of the region of the coronavirus's nucleotide sequence that corresponds to the target-variation region of the synthetic target-associated molecules to the peak intensities of the target variation region of the synthetic target-associated molecules.

In some embodiments, determining the presence or absence of the coronavirus comprises calculating relative abundances for the synthetic target-associated molecules and coronavirus nucleotide molecules by comparing the intensities across peaks for the synthetic target associated molecules and for the coronavirus's nucleotide sequence.

In some embodiments, the target variation region of the target-associated molecule comprises one or more deletions, wherein each deletion is 1-100 nucleotides (e.g. 1-2 nucleotides, 1-10 nucleotides, 1-25 nucleotides, 1-50 nucleotides, 1-4 nucleotide, 1-5 nucleotides, 25-50 nucleotides, 50-75 nucleotides, and the like). In some embodiments, the target variation region of the synthetic target-associated molecules comprise one or more insertions, wherein each insertion comprises 1-100 nucleotides.

In some embodiments, the coronavirus is selected from the group consisting of: coronavirus OC43, coronavirus 229E, coronavirus NL63, coronavirus HKU1, middle east respiratory syndrome beta coronavirus (MERS-CoV), severe acute respiratory syndrome beta coronavirus (SARS-CoV), and SARS-CoV-2.

In another aspect, the present disclosure provides a method of detecting the presence or absence of one or more infectious diseases from a sample obtained from a subject, the method comprising: generating a spike-in mixture including sample molecules from the sample and synthetic target-associated molecules, wherein the synthetic target-associated molecules comprise: a target-matching region that matches a corresponding nucleotide sequence in a first region of the infectious disease's nucleotide sequence, and a target-variation region that is distinguishable from a second region of the infectious disease's nucleotide sequence, the target-variation region having a nucleotide sequence with an insertion or deletion as compared to a corresponding nucleotide sequence in the second region of the infectious disease's nucleotide sequence; co-amplifying the spike-in mixture to generate a co-amplified spike-in mixture; performing capillary electrophoresis on the co-amplified spike-in mixture to generate a chromatogram-related output comprising a plurality of chromatogram intensities, the intensities including an intensity associated with: the synthetic target-associated molecules; and the sample molecules of the subject; and determining the presence or absence of at least one infectious disease based on the chromatogram intensities associated with the synthetic target-associated molecules and the sample molecules.

In some embodiments, determining the presence or absence of at least one infectious disease comprises comparing a peak intensity position associated with the synthetic target-associated molecules and a peak intensity position of the sample molecules of the subject, wherein the peak intensity position of the synthetic target-associated molecules is offset as compared to the peak intensity position of the sample molecules.

In some embodiments, performing capillary electrophoresis on the co-amplified spike-in mixture comprises sanger sequencing the co-amplified spike-in mixture.

In some embodiments, generating a spike-in mixture including sample molecules from the sample and synthetic target-associated molecules comprises: mixing the target-associated molecules with the sample molecules; and performing reverse transcription on the spike-in mixture to convert the sample molecules into DNA.

In some embodiments, the method does not consist of RNA extraction from the sample molecules suspected to contain the infectious disease.

In some embodiments, the chromatogram-related output comprises alignment positions corresponding to the chromatogram intensities, wherein the chromatogram intensities comprise peaks associated with: the target-matching region of the synthetic target-associated molecules; the target-variation region of the synthetic target-associated molecules; and the second region of the infectious disease's nucleotide sequence. In some embodiments, for each of the different pairs, the base of the nucleotide sequence of the synthetic target-associated molecule corresponds to a first alignment position that is different from a second alignment position corresponding to the base of the nucleotide sequence of the sample molecule, and wherein the alignment positions of the chromatogram-related output comprise the first and the second alignment positions.

In some embodiments, co-amplifying the spike-in mixture comprises amplifying the synthetic-target associated molecules and the sample molecules with a set of primers, wherein the set of primers include nucleotide sequences that are complementary or reverse complementary to the target matching region of the synthetic target-associated molecules and are complementary or reverse complementary to the first region of the infectious disease's nucleotide sequence.

In some embodiments, amplifying is performed using polymerase chain reaction (PCR).

In some embodiments, the method comprises, before co-amplifying, performing hybridization capture. In some embodiments, co-amplifying is performed using ligation amplification reaction (LAR). In some embodiments, the set of primers further comprise universal-tailed primers comprising universal tailed sequences. In some embodiments, the primers further comprise one or more fluorescently labeled tags. In some embodiments, the fluorescently labeled tags are attached at the 5′ end of the primer sequences. In some embodiments, the co-amplified mixture comprises synthetic target-associated amplicon products and, when the infectious disease is present in the sample, infectious disease amplicon products, the synthetic target-associated amplicon products comprising a nucleotide length that is shorter than the nucleotide length of the second region of the infectious disease's nucleotide sequence.

In some embodiments, the nucleotide length of the synthetic target-associated amplicon products is shorter by 1-50 nucleotides. In some embodiments, the co-amplified mixture comprises synthetic target-associated amplicon products, and, when the infectious disease is present in the sample, infectious disease amplicon products, the synthetic target-associated amplicon products comprising a nucleotide length that is longer than the nucleotide length of the second region of the infectious disease's nucleotide sequence. In some embodiments, the nucleotide length of the synthetic target-associated amplicon products is longer by 1-50 nucleotides.

In some embodiments, the peak associated with the second region of the infectious disease's nucleotide sequence includes a peak intensity position that is offset as compared to a peak intensity position of the target-variation region, the offset corresponding to the insertion or deletion of one or more nucleotides in the target-variation region. In some embodiments, the method further comprises determining an absolute abundance of infectious disease nucleotide molecules by comparing the intensity peaks of the second region of the infectious disease's nucleotide sequence to the intensity peaks of the target-variation region of the synthetic target-associated sample molecules, and wherein determining the absolute abundance is based on a known number of synthetic target-associated molecules added to the sample spike-in mixture.

In some embodiments, the chromatogram intensities comprise one or more fluorescence intensity peaks. In some embodiments, the method further comprises calculating the ratio of fluorescent intensity peaks of the sample amplicon products to the fluorescent intensity peaks of the synthetic target-associated amplicon products.

In some embodiments, the synthetic target-associated molecule is a DNA or RNA molecule. In some embodiments, the sample molecule is a DNA or RNA molecule.

In some embodiments, the infectious disease is: coronavirus, influenza virus, rhinovirus, respiratory syncytial virus, metapneumovirus, adenovirus, or boca virus. In some embodiments, the influenza virus is: parainfluenza virus 1, parainfluenza virus 2, influenza A virus, or influenza B virus. In some embodiments, the coronavirus is: coronavirus OC43, coronavirus 229E, coronavirus NL63, coronavirus HKU1, middle east respiratory syndrome beta coronavirus (MERS-CoV), severe acute respiratory syndrome beta coronavirus (SARS-CoV), or SARS-CoV-2.

In another aspect, the present disclosure provides A method of detecting the presence or absence of one or more infectious diseases in a sample obtained from a subject, the method comprising: generating a spike-in mixture including sample molecules from the sample and synthetic target-associated molecules, wherein the synthetic target-associated molecules comprise: a plurality of target-matching regions, each target matching region matching a corresponding nucleotide sequence in a first region of a corresponding infectious disease's RNA or DNA from a set of infectious diseases, and a plurality of target-variation regions, each target-variation region is distinguishable from a second region of the corresponding infectious disease's RNA or DNA from the set of infectious diseases, the target-variation region having a nucleotide sequence with an insertion or deletion as compared to a corresponding nucleotide sequence in the second region of the corresponding infectious disease's RNA or DNA from the set of infectious diseases; co-amplifying the synthetic target-associated molecules and sample molecules to generate a co-amplified spike-in mixture comprising amplicon products, wherein an amplicon product generated by amplifying a given infectious disease's RNA or DNA differs by a predetermined length from an amplicon product generated by amplifying the corresponding target matching and target variation regions of the synthetic target-associated molecules; performing capillary electrophoresis on the co-amplified spike-in mixture to determine a chromatogram-related output comprising a plurality of chromatogram intensities corresponding to the amplicon products; and determining the presence or absence of at least one infectious disease based on a chromatogram intensity associated with the amplicon product generated by amplifying the at least one infectious disease's RNA or DNA and a chromatogram intensity associated with an amplicon product having a length that differs by the predetermined length from the amplicon product generated by amplifying the at least one infectious disease's RNA or DNA.

In some embodiments, the synthetic target-associated molecules comprise: a first target-matching region that matches a corresponding nucleotide sequence in a first region of a first infectious disease's RNA or DNA, and a first target-variation region that is distinguishable from a second region of the first infectious disease's RNA or DNA, the target-variation region having a nucleotide sequence with an insertion or deletion as compared to a corresponding nucleotide sequence in the second region of the first infectious disease's RNA or DNA; a second target-matching region that matches a corresponding nucleotide sequence in a first region of the second infectious disease's RNA or DNA, and a second target-variation region that is distinguishable from a second region of the second infectious disease's RNA or DNA, the target-variation region having a nucleotide sequence with an insertion or deletion as compared to a corresponding nucleotide sequence in the second region of the second infectious disease's RNA or DNA.

In some embodiments, the synthetic target-associated molecules further comprise: a third target-matching region that matches a corresponding nucleotide sequence in a first region of the third infectious disease's RNA or DNA, and a third target-variation region that is distinguishable from a second region of the third infectious disease's RNA or DNA, the target-variation region having a nucleotide sequence with an insertion or deletion as compared to a corresponding nucleotide sequence in the second region of the third infectious disease's RNA or DNA.

In some embodiments, the amplicon products associated with the first infectious disease have a sample nucleotide length that is different by a second predetermined amount than that of sample amplicon products associated with the second infectious disease and of sample amplicon products associated with the third infectious disease.

In some embodiments, sets of primers used in co-amplification comprise a first set of primers including nucleotide sequences that are complementary to the first target matching region of the synthetic target-associated molecules and are complementary to the first region of the first infectious disease's RNA or DNA. In some embodiments, sets of primers comprise a second set of primers including nucleotide sequences that are complementary to the second target matching region of the synthetic target-associated molecules and are complementary to the first region of the second infectious disease's RNA or DNA. In some embodiments, sets of primers comprise a third set of primer including nucleotide sequences that are complementary to the third target matching region of the synthetic target-associated molecules and are complementary to the first region of the third infectious disease's RNA or DNA.

In some embodiments, the co-amplifying is performed using polymerase chain reaction (PCR), hybridization capture, or ligation amplification reaction (LAR). In some embodiments, the plurality of sets of primers further comprise universal-tailed primers comprising universal tailed sequences. In some embodiments, the co-amplified spike-in mixture comprises amplicon products of the synthetic target associated molecules and, when the corresponding infectious disease is present in the sample, amplicon products of the infectious disease's RNA or DNA.

In some embodiments, the synthetic target-associated amplicon products have a shorter nucleotide length as compared to amplicon products of the sample amplicon products by 1-100 nucleotides. In some embodiments, the synthetic target-associated amplicon products have a longer nucleotide length as compared to the amplicon products of the sample amplicon products by 1-100 nucleotides. In some embodiments, each set of primers comprises forward and reverse primer sequences. In some embodiments, the sets of primers comprise one or more fluorescently labeled tags.

In some embodiments, the synthetic target-associated amplicon products comprise a fluorescent label that is distinct in color from a fluorescent label of the amplicon products of the infectious disease's RNA or DNA.

In some embodiments, the synthetic target-associated amplicon products comprise a first set of target-associated amplicon products comprising the first target-matching region and the first target-variation region, and a second set of target-associated amplicon products comprising the second target-matching region and the second target-variation region, wherein the first set of target-associated amplicon products comprise a fluorescent label that is distinct from a fluorescent label of the second set of target-associated amplicon products.

In some embodiments, the amplicon products further comprise a first set of sample amplicon products for detecting a first infectious disease and a second set of sample amplicon products for detecting a second infectious disease, wherein the first set of sample amplicon products comprise a fluorescent label that is distinct from a fluorescent label of the second set of sample amplicon products.

In some embodiments, the first set of sample amplicon products and the first set of target-associated amplicon products comprise the same type of fluorescent label.

In some embodiments, the second set of sample amplicon products and the second set of target-associated amplicon products comprise the same type of fluorescent label.

In some embodiments, the forward and reverse primers comprise: (a) a first set of forward and reverse fluorescently labeled primers that are complementary to a nucleotide sequence corresponding to a first infectious disease; (b) a second set of forward and reverse fluorescently labeled primers that are complementary to a second infectious disease; and (c) a third set of forward and reverse fluorescently labeled primers that are complementary to a third infectious disease.

In some embodiments, the first set of forward and reverse fluorescently labeled primers comprise a fluorescent label that is distinct from a fluorescent label of the second set of forward and reverse fluorescently labeled primers. In some embodiments, the second set of forward and reverse fluorescently labeled primers comprise a fluorescent label that is distinct from a fluorescent label of the third set of forward and reverse fluorescently labeled primers. In some embodiments, the first set of forward and reverse fluorescently labeled primers comprise a fluorescent label that is distinct from a fluorescent label of the third set of forward and reverse fluorescently labeled primers.

In some embodiments, the fluorescent labels are attached at the 5′ end of the primer sequences.

In some embodiments, the chromatogram intensities comprise one or more intensity peaks. In some embodiments, the chromatogram intensities comprise one or more fluorescence intensity peaks. In some embodiments, the one or more intensity peaks of the synthetic target-associated amplicon products is associated with the target-associated nucleotide length, and wherein the one or more intensity peaks of the sample amplicon products is associated with the sample nucleotide length.

In some embodiments, the method further comprising calculating the ratio of intensity peaks of the sample amplicon products to the intensity peaks of the synthetic target-associated amplicon products. In some embodiments, the intensity peak of the region of the sample amplicon products that corresponds to the target-variation region of the synthetic target-associated amplicon products includes a peak intensity position that is offset as compared to the peak intensity position of the target-variation region, wherein the peak intensity position is offset by one or more nucleotides associated with the insertion or deletion of the target-variation region.

In some embodiments, determining the presence or absence of the infectious disease comprises comparing the chromatogram intensities comprises comparing a location of the intensity peak associated with the first target-variation region of the synthetic target-associated amplicon products and a location of the intensity peak of the region of the sample amplicon products of the subject. In some embodiments, determining the presence or absence of the infectious disease comprises comparing the chromatogram intensities comprises calculating the ratio between the intensity peak associated with the first target-variation region of the synthetic target-associated amplicon products and intensity peak of the region of the sample amplicon products of the subject.

In some embodiments, the method further comprises aggregating peak intensities across each synthetic target-associated amplicon products of the same nucleotide length; aggregating peak intensities across each sample amplicon product of the same nucleotide length; and comparing the aggregated peak intensities of the target-associated amplicon products and the sample amplicon products.

In some embodiments, the method further comprises computing a ratio between the aggregated sample amplicon product peak intensity and the aggregated synthetic target-associated amplicon product peak intensity.

In some embodiments, the target-associated molecule is a DNA or RNA molecule. In some embodiments, a first target variation region of the synthetic target-associated molecule comprises one or more deletions, wherein each deletion is 1-10 nucleotides. In some embodiments, a first target variation region of the synthetic target-associated molecule comprises one or more insertions, wherein each insertion is comprises 1-100 nucleotides.

In some embodiments, the one or more infectious diseases include one or more of: coronavirus, influenza virus, rhinovirus, respiratory syncytial virus, metapneumovirus, adenovirus, or boca virus. In some embodiments, the influenza virus is: parainfluenza virus 1, parainfluenza virus 2, influenza A virus, or influenza B virus. In some embodiments, the coronavirus is: coronavirus OC43, coronavirus 229E, coronavirus NL63, coronavirus HKU1, middle east respiratory syndrome beta coronavirus (MERS-CoV), severe acute respiratory syndrome beta coronavirus (SARS-CoV), or SARS-CoV-2.

In another aspect, the methods of the present disclosure provide a method of detecting the presence or absence of one or more infectious diseases in a sample obtained from a subject, the method comprising: generating a spike-in mixture including sample molecules from the sample and synthetic target-associated molecules, wherein the synthetic target-associated molecules comprise: a first target-matching region that matches a corresponding nucleotide sequence in a first region of a first infectious disease's RNA or DNA; and a target-variation region that is distinguishable from a second region of the first infectious disease's RNA or DNA, the target-variation region having a nucleotide sequence with an insertion or deletion as compared to a corresponding nucleotide sequence in the second region of the first infectious disease's RNA or DNA; co-amplifying the synthetic target-associated molecules and sample molecules from a subject with a set of primers to generate a co-amplified mixture of synthetic target-associated amplicon products, and sample amplicon products when the infectious disease is present in the sample, wherein co-amplifying the spike-in mixture comprises amplifying the synthetic target-associated molecules and the sample molecules with a set of primer sequences, wherein the set of primer sequences include nucleotide sequences that are complementary or reverse complementary to the first target matching region of the synthetic target-associated molecules and are complementary or reverse complementary to the first region of the first infectious disease's RNA or DNA, wherein the synthetic target-associated amplicon products have a target-associated nucleotide length that is different than a predetermined by a predetermined amount than a sample nucleotide length of the sample amplicon products; performing capillary electrophoresis on the co-amplified spike-in mixture to determine a chromatogram-related output comprising a plurality of chromatogram intensities, including an intensity associated with: amplicon products having the target-associated nucleotide length; and amplicon products having the sample nucleotide length; and determining the presence or absence of first infectious disease by comparing the chromatogram intensities associated with the amplicon products having the target-associated nucleotide length and amplicon products having the sample nucleotide length.

In some embodiments, amplifying is performed using polymerase chain reaction (PCR), hybridization capture, or ligation amplification reaction (LAR). In some embodiments, the set of primers further comprise universal-tailed primers comprising universal tailed sequences. In some embodiments, the amplicon products of the synthetic target-associated molecules have a shorter nucleotide length as compared to amplicon products of the sample molecule by 1-100 nucleotides.

In some embodiments, the amplicon products of the synthetic target-associated molecules have a longer nucleotide length as compared to the amplicon products of the sample molecule by 1-100 nucleotides. In some embodiments, the set of primer sequences comprise forward and reverse primer sequences. In some embodiments, the set of primers comprise one or more fluorescently labeled tags. In some embodiments, the synthetic target-associated amplicon products comprise a fluorescent label that is distinct from a fluorescent label of the sample amplicon products.

In some embodiments, the synthetic target-associated amplicon products comprise the target-matching region and the target-variation region, wherein the synthetic target-associated amplicon products comprise a fluorescent label that is distinct from a fluorescent label of the sample amplicon products. In some embodiments, the fluorescent labels are attached at the 5′ end of the primer sequences.

In some embodiments, the chromatogram intensities comprise one or more intensity peaks. In some embodiments, the chromatogram intensities comprise one or more fluorescence intensity peaks. In some embodiments, the one or more intensity peaks of the synthetic target-associated amplicon products is associated with a nucleotide length of the synthetic target-associated amplicon products, and wherein the one or more intensity peaks of the sample amplicon products is associated with a nucleotide length of the sample amplicon products.

In some embodiments, the method further comprises calculating comprises calculating the ratio of intensity peaks of the sample amplicon products to the intensity peaks of the synthetic target-associated amplicon products.

In some embodiments, the intensity peak of the region of the sample molecules that corresponds to the target-variation region of the synthetic target-associated molecules includes a peak intensity position that is offset as compared to the peak intensity position of the target-variation region, wherein the peak intensity position is offset by one or more nucleotides associated with the insertion or deletion of the target-variation region.

In some embodiments, determining the presence or absence of the infectious disease comprises comparing the chromatogram intensities by comparing a location of the intensity peak associated with the first target-variation region of the synthetic target-associated amplicon products and a location of the intensity peak of the region of the sample amplicon products of the subject.

In some embodiments, the method further comprises comparing the chromatogram intensities comprises calculating the ratio between the intensity peak associated with the first target-variation region of the synthetic target-associated amplicon products and intensity peak of the region of the sample amplicon products of the subject.

In some embodiments, comparing further comprises: aggregating peak intensities across each synthetic target-associated amplicon products of the same nucleotide length; aggregating peak intensities across each sample amplicon product of the same nucleotide lengths, and comparing the aggregated peaks intensities.

In some embodiments, the method further comprises computing a ratio between the aggregated sample amplicon product peak intensity and the aggregated synthetic target-associated amplicon product peak intensity.

In some embodiments, the target-associated molecule is a DNA or RNA molecule. In some embodiments, the nucleic acid molecule is a DNA or RNA molecule.

In some embodiments, the chromatogram intensities comprise one or more peak intensities associated with: the target-associated region of the target associated amplicon products; the target variation region of the target associated amplicon products; or the target region of the nucleic acid amplicon products of the subject.

In some embodiments, the first target variation region of the synthetic target-associated molecule comprises one or more deletions, wherein each deletion is 1-10 nucleotides. In some embodiments, the first target variation region of the synthetic target-associated molecule comprises one or more insertions, wherein each insertion is comprises 1-100 nucleotides.

In some embodiments, the infectious disease is: coronavirus, influenza virus, rhinovirus, respiratory syncytial virus, metapneumovirus, adenovirus, or boca virus. In some embodiments, the influenza virus is: parainfluenza virus 1, parainfluenza virus 2, influenza A virus, or influenza B virus. In some embodiments, the coronavirus is: coronavirus OC43, coronavirus 229E, coronavirus NL63, coronavirus HKU1, middle east respiratory syndrome beta coronavirus (MERS-CoV), severe acute respiratory syndrome beta coronavirus (SARS-CoV), or SARS-CoV-2.

### 4. BRIEF DESCRIPTION OF THE SEVERAL VIEWS OF THE DRAWINGS

These and other features, aspects, and advantages of the present disclosure will become better understood with regard to the following description, and accompanying drawings, where:

FIGS. 1A-1C show flow charts of various aspects of the present disclosure of sample preparation and applying a quantitative sanger sequencing (qSanger) approach during capillary electrophoresis for detecting sample molecules from the sample. FIG. 1C compares a qSanger workflow for an infectious disease, such as COVID-19, to qPCR, according to one embodiment.

FIGS. 2A-2D show a schematic illustration of a qSanger COVID-19 assay, according to one embodiment. (FIG. 2A) Specimen processing workflow. Reverse-transcription (RT) and PCR amplification of a SARS-CoV-2 target region is accomplished by directly addition of Viral Transport Media (VTM) to a one-step RT-PCR master mix containing ˜200 copies of synthetic spike-in DNA. The SARS-CoV-2 target region and spike-in DNA are co-amplified on a standard thermal cycler, and the amplification products are Sanger sequenced. Custom data analysis of the resulting chromatogram is then used to determine whether the specimen is COVID-19 negative or positive. (FIG. 2B) Synthetic spike-in is designed with sequence homology to the SARS-CoV-2 target so that it co-amplifies with the SARS-CoV-2 target. A 4-base pair (bp) deletion in the spike-in design enables quantification of relative abundances of spike-in and SARS-CoV-2 DNA from a Sanger sequencing chromatogram. The depicted forward and reverse primers are not to scale. (FIG. 2C) Representative Sanger sequencing traces showing pure genomic sequence (top), pure spike-in sequence (middle), and sequencing from a mixture of genomic and spike-in sequences (bottom). The spike-in used has a 4-bp offset compared to wild type (wt), which means that when two sequences are present, the signal from each sequence can be used to estimate their relative abundances (see arrows for examples of paired bases). (FIG. 2D) Representative genomic sequences corresponding to infectious diseases, synthetic target-associated sequences (spike in sequence) primers sequences, and sanger sequences.

FIG. 3A-3E show representative Sanger sequencing chromatograms for amplified products of spike-in DNA only, SarS-CoV-2 RNA only, or a mixture of the two, according to one embodiment. Since the spike-in is an internal control for amplification and sequencing, observing the spike-in signal only indicates that SARS-CoV-2 RNA was absent, and therefore the specimen is negative for COVID-19. In contrast, observing SARS-CoV-2 signal only indicates that SARS-COV-2 RNA was so abundant in the specimen that it is above the quantifiable range. In a mixed chromatogram of spike-in and SARS-CoV-2, the abundance of SARS-CoV-2 RNA is determined by the relative contributions of SARS-CoV-2 and spike-in signal intensities. (FIG. 3B) Additional data analysis and interpretation of qSanger COVID-19 assay. Representative Sanger sequencing chromatograms are shown for amplified products of a sample molecule that is positive for COVID-19, synthetic DNA only that is negative for COVID-19, an inconclusive result where there was a PCR failure, and an inconclusive result where there was an RNA extraction failure. (FIG. 3C—3E) Synthetic viral genomic RNA was added to RT-PCR reactions at 10, 100, 1000, and 5000 GCE. The same dilutions were subjected to qSanger testing and RT-qPCR. (FIG. 3C) RT-qPCR exhibits a linear estimate across the dilution series, consistent with previous results. (FIG. 3D) Across the same dilution series, the ratio (R) of genomic sequence to spike-in sequence scales with RNA added. (FIG. 3E) When the qPCR estimate of abundance is compared to qSanger estimates of abundance, they exhibit a strong linear relationship, indicating that qSanger performs as well as qPCR in estimates of viral RNA abundance.

FIG. 4A-4B provides an example where qSanger detects SARS-COV-2 RNA when amplified directly from viral particles in transport medium, according to one embodiment. (FIG. 4A) A total of 32 no-template controls, 32 negative control samples (Seracare) and 32 positive samples (Seracare) were assayed. All results were concordant with Seracare and NTC. Three samples were no-calls (undetermined) due to low signal-to-noise ratio in the sequencing results. (FIG. 4B) Positive Seracare samples were added to RT-PCR master mix either directly from the VTM or after purification with RNA extraction kit at 125 GCE. The ratio of reference and spike-in intensities were measured by custom data analysis. The mean qSanger ratio was 0.745 (±0.043 s.e.m., n=8) for direct addition, and 0.97 (±0.041 s.e.m., n=8) for purified. The coefficient of variation (CV) of positive seracare samples were measured for both Luna and OneTaq polymerase mixes. The CV for Luna direct VTM was 16.4% (n=8), and for Luna purified was 12.1% (n=8). This is consistent with the theoretical counting noise associated with quantifying ˜100 molecules.

FIG. 5A-5B shows that qSanger detects as little as 20 GCE without RNA purification, according to one embodiment. (FIG. 5A) Representative Sanger sequencing traces of negative control virus (left) and SARS-CoV-2 sequence containing virus (right). Even when the signal is too low to detect mixed bases, the 3′ offset caused by the deletion in the spike-in compared to the genomic sequence identifies positive samples. The sequencing peaks identified in the inset correspond to spike-in sequence offset by 4 bp (see paired arrows). (FIG. 5B) Twenty samples each of negative control virus and SARS-CoV-2 sequence containing virus were directly added to RT-PCR master mix with 100 spike-in molecules and Sanger sequencing was performed. All samples that successfully sequenced were accurately identified (one negative sample was undetermined due to sequencing failure).

FIG. 6 shows an example of a chromatogram representing intensity signals of a synthetic target-associated molecule, according to one embodiment. AccuPlex SARS-CoV-2 Negative Reference Material (only spike-in sequence should be present).

FIG. 7 shows an example of a chromatogram representing intensity signals of a sample that contains coronavirus, according to one embodiment. AccuPlex SARS-CoV-2 Positive Reference Material (mixed sequence should be present along with a 4 bp tail—see the repeat of black (G), blue (C), blue (C), green (A) at the 3′ tail).

FIG. 8 shows an example of a strongly positive result (e.g., presence of the infectious disease in the sample), according to one embodiment.

FIG. 9 shows an example of a weakly positive result (weak presence of the infectious disease in the sample), according to one embodiment. Note the mixed sequence highlighted above as well as the 3′ viral sequence. Note that the 3′ viral sequence alone is sufficient to indicate the presence of viral genomic sequence.

FIG. 10 shows a chromatogram distinguishing purely viral sequence (top) from purely spike-in sequence (bottom), according to one embodiment. Note the 4 missing bases in the bottom panel as compared to the top.

FIG. 11A-11D provides a schematic illustration of the steps of preparation and fragment analysis of an infectious disease assay, according to one embodiment. FIGS. 11A-11B provide flow charts of active steps of the fragment analysis procedure, including amplification/capture, labeling of the molecules, and performing capillary electrophoresis. FIG. 11C provides a flow chart of a multiplex approach of labeling and analyzing a plurality of molecules containing one or more infectious diseases in a single assay. FIG. 11D provides a flow chart illustration of the steps for preparation and fragment analysis of COVID-19.

FIG. 12 provides an illustration of a fluorescent labeling approach for detecting the presence or absence of one or more infectious diseases in a single assay, according to one embodiment.

FIG. 13 provides the specific nucleotide sequences of the target molecules and spike-in molecules of interest for specific infectious diseases, and the primer design for co-amplification, according to one embodiment.

FIG. 14 shows peaks that are distinguished by two varying nucleotide lengths: those less than 25 bp and those that are within the 90-120 bp range, according to one embodiment. The peaks at 25 bp or less are the result of residual unincorporated labeling primers, which are 20 bp each. The size variability of these peaks below 25 bp is not unexpected since this is below the range of the size standard, which has fragments that are 35-500 bp in length. The 6 cycle labeling method notably has a higher number of peaks measuring below 25 bp as compared to the 30 cycle labeling method. This means that the 6 cycle labeling approach has more residual labeling primers. For downstream analysis, peak data was filtered to remove peaks from the unincorporated primer. Peaks measuring in the 90-120 bp range are from the labeled target molecules, which are designed to be 96-120 bp. For peaks within this range, the observed signal intensity varies per sample. Samples labeled with the 30 cycle method appeared to have higher maximum intensities than the 6 cycle labeling approach, which is expected given the trends in residual unincorporated labeling primer across these two methods.

FIG. 15 shows how peaks are distinguished and labeled by size (e.g., length of nucleotides) in a chromatogram, according to one embodiment. Spike-ins differed from reference sequence by a 4 bp deletion, resulting in a staggered qSanger-like peak arrangement. The processed peak data outputs both the beginning and ending points of the detected peak, documented as data points or scan numbers where one base pair is approximately 20 data points. The difference in data points of consecutive peaks was calculated as shown in FIG. 15. Consecutive peaks had a minimum separation of 8 data points, meaning none of the detected target peaks overlapped with another and a 4 bp deletion gives sufficient separation. The peaks are thus all clearly resolved and can be treated independently in downstream calculations.

FIG. 16 a measurement of assay pooled shot noise, according to one embodiment. Shot noise was measured by injecting many technical replicates (n=24) on a single plate. Technical replicates were prepared by pooling products across all replicates of a labeling reaction condition. This pooled labeled product was combined with the size standard and diluted in formamide. The sample and size standard mixture was aliquotted into 24 wells of a plate for injection. The two different FAMF labeling reactions (30 cycle and 6 cycle) on the singleplex (60 bp amplicon) sample were used to assess the shot noise. The reference to spike-in ratios were calculated using three different peak values: peak area in base pairs, peak area in data points, and peak height. The CV for each of these reference to spike-in ratio types is shown.

FIG. 17 provides a graph showing the distribution of peak intensities for each labeling method, according to one embodiment. The higher shot noise in samples labeled using 6 cycles could potentially be explained by the difference in absolute intensities seen across the two labeling methods. The 30-cycle labeling method resulted in peaks of higher intensities as compared to the 6 cycle labeling method. If shot noise were a function of intensity, the systematically higher shot noise for lower signals would be explained. To confirm this hypothesis, and additional experiment would need to be run using a sample injected on a dilution gradient to control for sample composition.

FIG. 18 shows data representing the results of a noise assay test, according to one embodiment. Noise was assessed using 16 replicates of samples labeled either with the forward or reverse FAM primer at 6 or 30 cycles. Labeling template consisted of amplified product containing amplicons of 60 bp or 60 bp and 80 bp in length. Amplified product was pooled prior to labeling to eliminate noise from the initial amplification reaction. Labeled products were combined with a size standard and denatured in formamide for injection. Reference to spike-in ratios were calculated using both area and height data for the detected peaks. The CV of the reference to spike-in ratio per tested condition is shown.

### 5. DETAILED DESCRIPTION

5.1. Methods of Detecting the Presence or Absence of Infectious Diseases

Aspects of the present disclosure include methods of detecting the presence or absence of one or more infectious diseases from a sample obtained from a subject.

In one aspect, the method includes generating a spike-in mixture including sample molecules from the sample (e.g., “target sequence”, “target molecule” “target sample”) and synthetic target-associated molecules (e.g., “spike-in molecule”, “spike-in reference molecule”), co-amplifying the spike-in mixture to generate a co-amplified spike-in mixture, performing capillary electrophoresis on the co-amplified spike-in mixture to generate a chromatogram-related output, and determining the presence or absence of one or more infectious diseases by comparing the intensities associated with the first target-variation region of the synthetic target associated molecules and the region of the sample molecules of the subject that corresponds to the target-variation region of the synthetic target-associated molecules.

Embodiments of the synthetic target-associated molecules of the present methods include a target-matching region that matches a corresponding nucleotide sequence in a first region of the infectious disease's RNA or DNA, and a target-variation region that is distinguishable from a second region of the infectious disease's RNA or DNA, the target-variation region having a nucleotide sequence with an insertion or deletion as compared to a corresponding nucleotide sequence in the second region of the infectious disease's RNA or DNA RNA.

As used herein, the term “match” can include a sequence that has similar or 100% sequence identity to the nucleotide sequence in a first region of the infectious disease's RNA or DNA, a DNA complement or reverse complement of the nucleotide sequence in a first region of the infectious disease's RNA or DNA, or a RNA complement or reverse complement of the nucleotide sequence in a first region of the infectious disease's RNA or DNA.

The target-matching region that matches a corresponding nucleotide sequence in a first region of the infectious disease's RNA or DNA shares one or more characteristics (e.g., sequence characteristics, functional characteristics, structural characteristics, evolutionary characteristics, etc.) with the first region of the infectious disease's RNA or RNA (e.g., biological targets; etc.).

5.1.1. Infectious Diseases

Aspects of the present disclosure include methods of detecting one or more infectious diseases.

The infectious diseases that can be detected using the methods of the present disclosure include: coronavirus, influenza virus, rhinovirus, respiratory syncytial virus, metapneumovirus, adenovirus, boca virus, or any other infectious disease.

In some embodiments, the infectious disease is a respiratory disease. In some embodiments, the infectious disease is a bacterial infection. In some embodiments, the infectious disease is a sexually transmitted disease or another infectious disease. In some embodiments, the infectious disease is any pathogen with DNA genomes. In some embodiments, the disease is caused by herpes simplex-1 virus (HSV-1), herpes simplex-2 virus (HSV-2), human immunodeficiency virus (HIV), HIV-2 Group A, HIV-2 Group B, HIV-1 Group M, Hepatitis B, Hepatitis Delta, herpes simplex virus (HSV), streptococcus B, and Treponema pallidum. In some embodiments, the infectious disease is selected from: Influenza A Matrix protein, Influenza H3N2, Influenza H1N1 seasonal, Influenza H1N1 novel, Influenza B, an Ebola virus, a Marburg virus, a Cueva virus, Streptococcus pyogenes (A), Mycobacterium Tuberculosis, Staphylococcus aureus (MR), Staphylococcus aureus (RS), Bordetella pertussis (whooping cough), Streptococcus agalactiae (B), Influenza H5N1, Influenza H7N9, Adenovirus B, Adenovirus C, Adenovirus E, Hepatitis b, Hepatitis c, Hepatitis delta, Treponema pallidum, HSV-1, HSV-2, HIV-1, HIV-2, Dengue 1, Dengue 2, Dengue 3, Dengue 4, Malaria, West Nile Virus, Trypanosoma cruzi (Chagas), Klebsiella pneumoniae (Enterobacteriaceae spp), Klebsiella pneumoniae carbapenemase (KPC), Epstein Barr Virus (mono), Rhinovirus, Parainfluenza virus (1), Parainfluenza virus (2), Parainfluenza virus (3), Parainfluenza virus (4a), Parainfluenza virus (4b), Respiratory syncytial virus (RSV) A, Respiratory syncytial virus (RSV) B, Coronavirus 229E, Coronavirus HKU1, Coronavirus OC43, coronavirus OC43, coronavirus 229E, coronavirus NL63, coronavirus HKU1, middle east respiratory syndrome beta coronavirus (MERS-CoV), severe acute respiratory syndrome beta coronavirus (SARS-CoV), and SARS-CoV-2, Coronavirus NL63, Novel Coronavirus, Bocavirus, human metapneumovirus (HMPV), Streptococcus pneumoniae (penic R), Streptococcus pneumoniae (S), Mycoplasma pneumoniae, Chlamydia pneumoniae, Bordetella parpertussis, Haemophilus influenzae (ampic R), Haemophilus influenzae (ampic S), Moraxella catarrhalis, Pseudomonas spp (aeruginosa), Haemophilus parainfluenzae, Enterobacter cloacae (Enterobacteriaceae spp), Enterobacter aerogenes (Enterobacteriaceae spp), Serratia marcescens (Enterobacteriaceae spp), Acinetobacter baumanii, Legionella spp, Escherichia coli, Candida, Chlamydia trachomatis, Human Papilloma Virus, Neisseria gonorrhoeae, plasmodium, and Trichomonas (vagin).

In some embodiments, the infectious disease is tuberculosis (Mycobacterium tuberculosis). In some embodiments, the disease is associated with a Staphylococcus bacterium or a Streptococcus bacterium.

In some embodiments, the infectious disease is a virus selected from the group of viruses consisting of a filo virus, a Coronavirus, West Nile Virus, Epstein-Barr Virus, and a Dengue Virus.

In some embodiments, the infectious disease is a virus. In certain embodiments, the virus is an influenza virus selected from the group consisting of: parainfluenza virus 1, parainfluenza virus 2, influenza A virus, and influenza B virus.

In some embodiments, the virus is a coronavirus selected from the group consisting of: coronavirus OC43, coronavirus 229E, coronavirus NL63, coronavirus HKU1, middle east respiratory syndrome beta coronavirus (MERS-CoV), severe acute respiratory syndrome beta coronavirus (SARS-CoV), and SARS-CoV-2.

In certain embodiments, the coronavirus is SARS-CoV-2 (COVID-19). In certain embodiments, detection of the coronavirus can be any known coronavirus strain or variant.

5.1.2. Target Sample

The target sample of the present methods include samples obtained from the subject.

Collected samples (e.g., biological samples; collected using sample containers provided to users in sample collection kits) can include any one or more of: blood, plasma, serum, tissue, biopsies (e.g., tumor biopsies, etc.), sweat, urine, feces, semen, vaginal discharges, tears, interstitial fluid, respiratory mucosa, nasal mucosa, other body fluid, and/or any other suitable samples (e.g., associated with a human user, animal, object such as food, microorganisms, etc.). In certain embodiments, the samples include target molecules (e.g., nucleic acid molecules including one or more target sequences and/or target sequence regions; etc.) and/or reference molecules (e.g., nucleic acid molecules including one or more reference sequences and/or reference sequence regions; etc.), such as where the target molecules can be amplified with the target-associated molecules under similar parameters; where the reference molecules can be amplified with the reference-associated molecules under similar parameters; etc.). Additionally or alternatively, samples can include target sample molecules collected across multiple time periods, and/or components varying across any suitable condition, such that generating spike-in mixture(s) can be performed for any suitable number and type of entities.

In some embodiments where an infectious disease is found to be present in the target sample of the subject, the target molecule will include a nucleotide sequence that has at least 80% sequence identity, at least 85% sequence identity, at least 90% sequence identity, at least 95% sequence identity, at least 96% sequence identity, at least 97% sequence identity, at least 98% sequence identity, at least 99% sequence identity, or 100% sequence identity to a nucleotide sequence region of the infectious disease.

In some embodiments, the target sample molecule is a DNA molecule. In other embodiments, the target sample molecule is an RNA sample.

5.1.3. Synthetic Target Associated Molecules

The synthetic target-associated molecules of the present methods include a first target-matching region that matches a corresponding nucleotide sequence in a first region of the a first infectious disease's RNA or DNA; and a target-variation region that is distinguishable from a second region of the first infectious disease's RNA or DNA, the target-variation region having a nucleotide sequence with an insertion or deletion as compared to a corresponding nucleotide sequence in the second region of the first infectious disease's RNA or DNA.

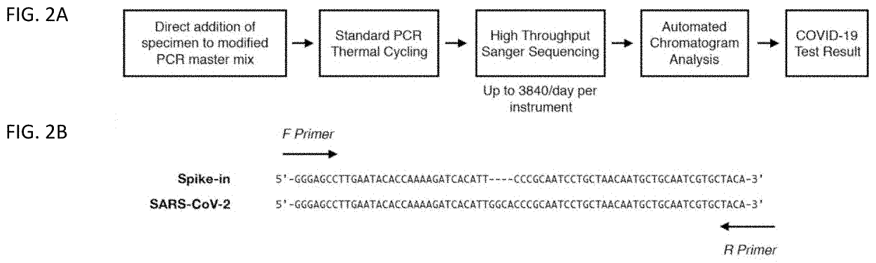

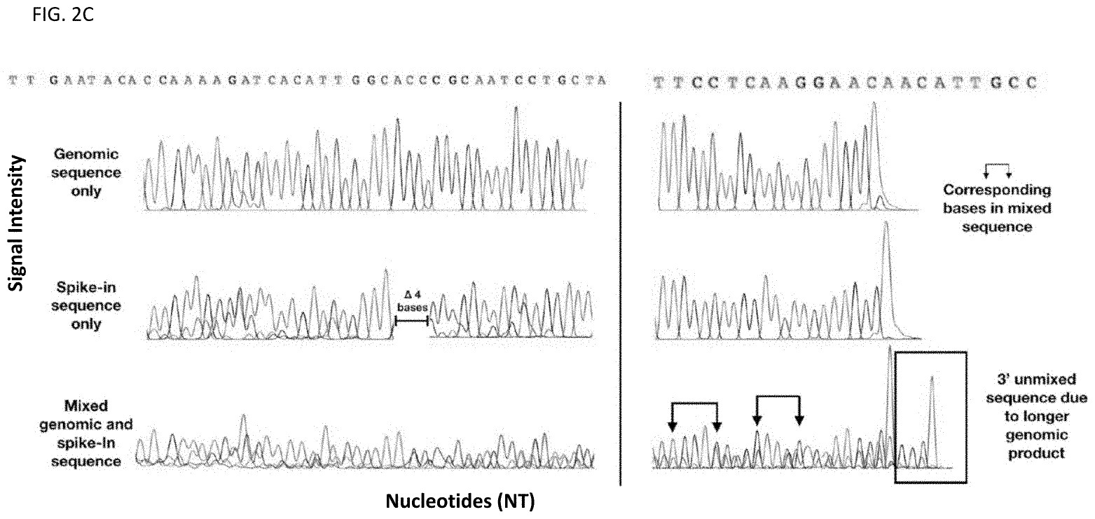

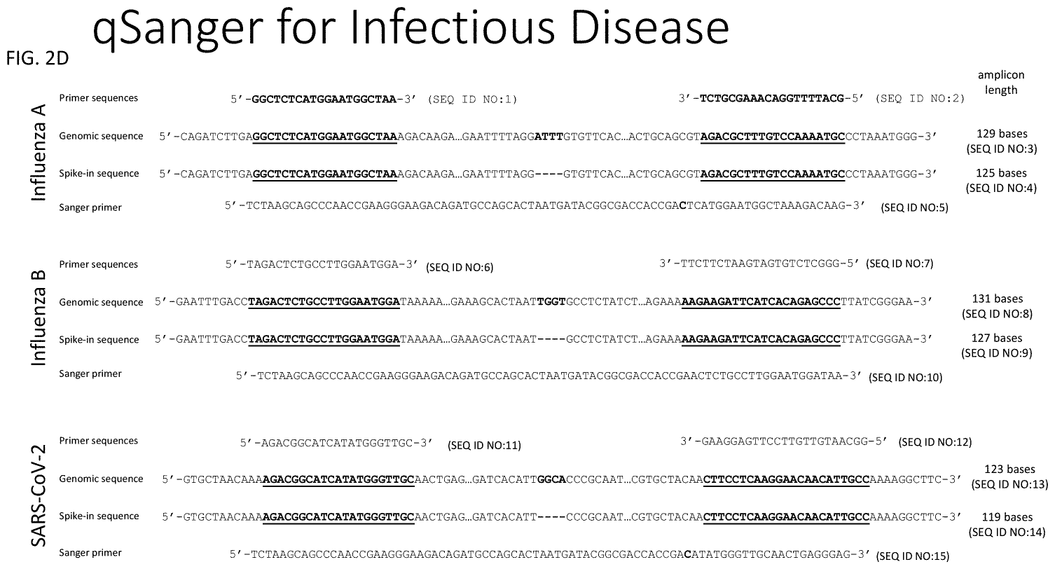

Embodiments of the method can include generating one or more synthetic target-associated molecules (e.g., “spike-in sequence” of FIG. 2D and FIG. 13, associated with one or more infectious disease biological targets, etc.), which can function to synthesize one or more molecules sharing one or more characteristics (e.g., sequence characteristics, functional characteristics, structural characteristics, evolutionary characteristics, etc.) with the one or more targets (e.g., biological targets; etc.), which can facilitate similar sample processing parameters (e.g., capillary electrophoresis, sanger sequencing conditions for sequencing of a spike-in mixture including the target-associated molecules and the target molecules, for sequencing the target-associated molecules individually or the target molecules individually; fragment analysis, similar amplification parameters during PCR-based amplification, such as through co-amplification of synthetic target-associated molecules and target molecules, of reference-associated molecules and reference molecules; etc.) to reduce bias (e.g., amplification bias, etc.) and/or to improve accuracy during downstream processing (e.g., for statistical estimation such as linear regression; peak analysis; association and/or identification of pairs and/or sets of bases of different sequence for facilitating abundance ratio determination; deconvolution; performance of multiple instances of embodiments of the method 100 over time; etc.).

Synthetic target-associated molecules can have one or more target-matching regions and one or more target-variation regions. For example, in a multiplexed method where the presence or absence of more than one infectious disease can be detected, the synthetic target associated molecules can subsets of synthetic target associated molecules, where each subset of synthetic target-associated molecules having a target matching region a target-variation region, each subset is used for targeting a different infectious disease. For example, the synthetic target associated sample molecules can include a first set of synthetic target associated sample molecules comprising a target-matching region and a target-variation region used in the spike-in mixture for detecting a first infectious disease, and a second set of synthetic target associated sample molecules comprising a target-matching region and a target-variation region for detecting a second infectious disease used in the same spike-in mixture. These subsets can be mixed together during co-amplification with the sample molecules.

Alternatively, each synthetic target-associated molecule can have a plurality of target-matching regions and target-variation regions on the same synthetic target-associated molecule, where each target-matching region and target-variation region set is for detecting a specific infectious disease for a multiplexed method. For example, the synthetic target associated sample molecules can include a first target-matching region and a first target-variation region for detecting a first infectious disease, and a second target-matching region and a second target-variation region for detecting a second infectious disease.

Moreover, in a singleplexed method where the presence or absence of a single infectious disease is being detected, the synthetic target-associated molecules will include a target-matching region and a target-variation region for detecting a single infectious disease. However, more than one target-matching region and target-variation region that is associated with the single infectious disease can be used to facilitate detection.

Synthetic target-associated molecules preferably include one or more target-associated sequences (e.g., nucleotide sequences; each target-associated molecule of a set of synthetic target-associated molecules corresponding to a same or similar target-associated molecule sequence; etc.), where a target-associated sequence can include one or more target-associated regions. For example, a target-associated sequence can include a target-associated region with sequence identity (e.g., 100% sequence identity, 99% sequence identity, 95% sequence identity, 90% sequence identity, 85% sequence identity, or 80% sequence identity, sequence similarity greater than a threshold percentage and/or amount; etc.) to one or more target sequence regions of one or more target sequences of one or more biological targets (e.g., a target DNA or RNA sequence corresponding to the biological target; etc.), where the one or more biological targets can be associated with one or more infectious diseases.

Synthetic target-associated regions (and/or the synthetic target-associated molecules and/or target-associated sequences) are preferably associated with (e.g., sharing nucleotide sequences with; sharing sets of bases with a target sequence at corresponding positions; able to be processed with; able to be Sanger sequenced with; able to be amplified with, such as through co-amplification; able to be targeted by the same primers; complementary to; targeting; digitally associated with in a computing system; etc.) one or more biological targets and/or target molecules (e.g., target molecules corresponding to biological targets; target molecules including target sequence regions of biological targets; etc.). Biological targets (e.g., target markers; corresponding to, causing, contributing to, therapeutic in relation to, correlated with, and/or otherwise associated with one or more infectious diseases; targets of interest; known or identified targets; unknown or previously unidentified targets; etc.) can include any one or more of target sequence regions (e.g., sequences identifying a chromosome; sequences indicative of a condition such as a virus; sequences that are invariant across a population and/or any suitable set of subjects; conserved sequences; sequences including mutations, polymorphisms; nucleotide sequences; amino acid sequences; etc.), genes (e.g., associated with one or more single gene disorders, etc.), loci, chromosomes (e.g., associated with one or more chromosomal abnormalities; etc.) nucleic acids encoding: proteins (e.g., serum proteins, antibodies, etc.), peptides, carbohydrates, and lipids, nucleic acids (e.g., extracellular RNA, microRNA, messenger RNA, where abundance determination for RNA targets can include suitable reverse transcriptase operations, etc.), cells (e.g., whole cells, etc.), metabolites, natural products, cancer biomarkers (e.g., molecules secreted by tumors; molecules secreted in response to presence of cancer; etc.), genetic predisposition biomarkers, diagnostic biomarkers, prognostic biomarkers, predictive biomarkers, other molecular biomarkers, gene expression markers, imaging biomarkers, and/or other suitable targets. Targets are preferably associated with conditions described herein, and can additionally or alternatively be associated with one or more conditions including: symptoms, causes, diseases, disorders, and/or any other suitable aspects associated with conditions. In an example, synthetic target-associated molecules can include nucleotide sequences identical to one or more regions of a target sequence of a target molecule (e.g., identifying SARS-CoV-2), where primers can concurrently target both the synthetic target-associated molecules and the target sample molecules by targeting the identical regions (e.g., for facilitating co-amplification, such as to reduce amplification bias, etc.). In an example, as shown in FIG. 2 and FIG. 13, a synthetic target-associated sequence (e.g., “spike-in” sequence), can include target-associated regions with sequence similarity to target sequence regions of the target sequence (e.g., “Genomic” sequence), such as where a set of primers (e.g., for a first PCR process, for a second PCR process, PCR primers including one or more hairpin sequences; etc.) can target both the synthetic target-associated sequence and the target sample sequence (e.g., for facilitating co-amplification and corresponding reduction of amplification biases; etc.).

In an example, synthetic target-associated molecules can include sequences with any suitable sequence identity to target sequences, where any number and/or type of primers can be used in concurrently or separately targeting the synthetic target-associated molecules and target molecules. In a specific example, the biological targets can include target sequences identifying an influenza virus or a coronavirus corresponding to a viral condition. In a specific example, the biological targets can include target sequences identifying viral DNA or RNA (e.g., in relation to determining virus condition metrics, evaluating virus treatments, etc.). However, targets (e.g., biological targets, etc.) can be configured in any suitable manner. Additionally or alternatively, synthetic target-associated molecules (e.g., target-associated regions of synthetic target-associated molecules; etc.) can share any suitable characteristics (e.g., components, etc.) with biological targets (e.g., with target molecules corresponding to biological targets; etc.), such as to facilitate similar sample processing parameters to be able to subsequently generate meaningful comparisons between abundance metrics for the synthetic target-associated molecules and the target molecules. However, synthetic target-associated molecules can be configured in any suitable manner.

As shown in FIG. 2 and FIG. 13, synthetic target-associated molecules preferably include target variation regions (e.g., variation regions of synthetic target-associated sequences of synthetic target-associated molecules; each synthetic target-associated molecule including one or more variation regions; etc.), where a variation region can include different characteristics from the characteristics of the target sample molecule. Variation regions preferably include one or more variations (e.g., insertions, deletions, substitutions, etc.), such as variations that can enable a corresponding synthetic target-associated molecule (e.g., the synthetic target-associated molecule including a target-associated sequence including the variation region; etc.) to proceed through sample processing operations in a similar manner to the corresponding target sample molecules (e.g., nucleic acids including a target sequence region of a biological target; etc.), while facilitating differentiation of the synthetic target-associated molecules from the target molecules (e.g., during determining of sequencing outputs; during determination of abundance metrics, such as performing statistical estimation analysis; for facilitating characterizations and/or treatments of one or more medical conditions; during post-processing of chromatogram-related outputs from capillary electrophoresis of spike-in mixtures and/or suitable samples, such as (in the case of using sanger sequencing) during deconvolution of overlapping peaks for pairs of target-associated base and target base, such as pairs corresponding to positions of variation regions and/or other suitable regions; during statistical estimation analyses to fit the abundances for target-associated sequences and target sequences; etc.). Such differentiation can facilitate determination of different corresponding abundance metrics that can be meaningful compared (e.g., quantitative comparison between peak intensities of pairs and/or sets of bases; comparison and/or combination of individual abundance metrics, such as to determine overall abundance metrics; such as for facilitating characterization and/or treatment; etc.) In an example, the variation region can include a sequence variation region including a nucleotide sequence differing from a sequence region of a target sequence of a target molecule. In a specific example, as shown in FIG. 2 and FIG. 13, a target-associated sequence (e.g., “spike-in” sequence; etc.), can include a deletion (e.g., a three nucleotide deletion; a four nucleotide deletion, a five-nucleotide deletion, a six nucleotide deletion, a seven nucleotide deletion, a eight nucleotide deletion, a nine nucleotide deletion, a ten nucleotide deletion, etc.) relative a sequence region of the target sample sequence (e.g., relative an “ATTT” sequence region of the “influenza A” target sequence; relative an “TGGT” sequence region of the “influenza B” target sequence; relative an “GGCA” sequence region of “SARS-CoV-2” target sequence, etc.). Additionally or alternatively, variation regions can include any suitable number of substitutions, insertions, deletions, and/or other modifications of any suitable size (e.g., insertions and/or deletions of any suitable number of nucleotides; any suitable number of point mutations, such as to point mutations; etc.) in relation to any suitable bases and/or base types.

In some embodiments, the target variation region of the synthetic-target associated sequence comprises 1-100 nucleotide deletion, such as (1-4 nucleotide deletion, 1-2 nucleotide deletion, 1-3 nucleotide deletion, 1-10 nucleotide deletion, 1-20 nucleotide deletion, 1-50 nucleotide deletion, 5-10 nucleotide deletion, 1-5 nucleotide deletion, 10-50 nucleotide deletion, 50-75 nucleotide deletion, 75-100 nucleotide deletion, and the like). In certain embodiments, the target variation region comprises a single nucleotide deletion. In certain embodiments, the target variation region comprises a 2 nucleotide deletion. In certain embodiments, the target variation region comprises a 3 nucleotide deletion. In certain embodiments, the target variation region comprises a 4 nucleotide deletion. In certain embodiments, the target variation region comprises a 5 nucleotide deletion. In certain embodiments, the target variation region comprises a 6 nucleotide deletion. In certain embodiments, the target variation region comprises a 7 nucleotide deletion. In certain embodiments, the target variation region comprises a 8 nucleotide deletion. In certain embodiments, the target variation region comprises a 9 nucleotide deletion. In certain embodiments, the target variation region comprises a 10 nucleotide deletion.

In some embodiments, the target variation region of the synthetic-target associated sequence comprises 1-100 nucleotide insertion, such as (1-4 nucleotide insertion, 1-2 nucleotide insertion, 1-3 nucleotide insertion, 1-10 nucleotide insertion, 1-20 nucleotide insertion, 1-50 nucleotide insertion, 5-10 nucleotide insertion, 1-5 nucleotide insertion, 10-50 nucleotide insertion, 50-75 nucleotide insertion, 75-100 nucleotide insertion, and the like). In certain embodiments, the target variation region comprises a single nucleotide insertion. In certain embodiments, the target variation region comprises a 2 nucleotide insertion. In certain embodiments, the target variation region comprises a 3 nucleotide insertion. In certain embodiments, the target variation region comprises a 4 nucleotide insertion. In certain embodiments, the target variation region comprises a 5 nucleotide insertion. In certain embodiments, the target variation region comprises a 6 nucleotide insertion. In certain embodiments, the target variation region comprises a 7 nucleotide insertion. In certain embodiments, the target variation region comprises a 8 nucleotide insertion. In certain embodiments, the target variation region comprises a 9 nucleotide insertion. In certain embodiments, the target variation region comprises a 10 nucleotide insertion.

In some embodiments, the target variation region of the synthetic-target associated sequence comprises 1-100 nucleotide substitution, such as (1-4 nucleotide substitution, 1-2 nucleotide substitution, 1-3 nucleotide substitution, 1-10 nucleotide substitution, 1-20 nucleotide substitution, 1-50 nucleotide substitution, 5-10 nucleotide substitution, 1-5 nucleotide substitution, 10-50 nucleotide substitution, 50-75 nucleotide substitution, 75-100 nucleotide substitution, and the like). In certain embodiments, the target variation region comprises a single nucleotide substitution. In certain embodiments, the target variation region comprises a 2 nucleotide substitution. In certain embodiments, the target variation region comprises a 3 nucleotide substitution. In certain embodiments, the target variation region comprises a 4 nucleotide substitution. In certain embodiments, the target variation region comprises a 5 nucleotide substitution. In certain embodiments, the target variation region comprises a 6 nucleotide substitution. In certain embodiments, the target variation region comprises a 7 nucleotide substitution. In certain embodiments, the target variation region comprises a 8 nucleotide substitution. In certain embodiments, the target variation region comprises a 9 nucleotide substitution. In certain embodiments, the target variation region comprises a 10 nucleotide substitution.

In a specific example, the variation regions can facilitate determination of sequencing outputs (e.g., peak intensities, peak area, peak data, chromatograms, etc.) for any target-associated base (e.g., of a target-associated sequence; etc.) and/or target base (e.g., of a target sequence; etc.), such as where a sequencing output (e.g., peak intensity metric) for a target-associated base at one or more regions (e.g., a target-associated region, a variation region, etc.) can be compared to a sequencing output (e.g., peak intensity metric, etc.) for a corresponding target base at a different position (e.g., where a position of a corresponding base can be shifted due to one or more insertions and/or deletions of a variation region; etc.) or same position (e.g., for point substitutions of a variation region; etc.), such as for determining one or more abundance metrics.

Variation regions can be designed in coordination with the synthetic target-associated regions to facilitate appropriate sequence dissimilarity and sequence similarity, respectively (e.g., determining characteristics of the variation regions and/or target-associated regions to facilitate improved sequencing outputs given sequencing parameters associated with the sequencing technologies, such as Sanger sequencing; etc.).

Sequence variation regions can differ by target sequences by any suitable number and type of bases, at any suitable positions (e.g., sequential positions, non-sequential; etc.), across any suitable loci, for any suitable chromosome and/or other target, and/or can differ from target sequences in any suitable manner. Sequence variation regions can include any one or more of substitutions, insertions, deletions, any suitable mutation types, and/or any suitable modifications (e.g., relative one or more sequence regions of a target sequence and/or biological target; etc.).

In a variation, sequence variation regions can include randomly shuffled bases (e.g., in equal proportion of base types, in predetermined portions for the base types, etc.). In a variation, the method 100 can include selecting bases of the target sequence to modify (e.g., based on optimizing Sanger sequencing output results, such as through selecting a specific sequence of base types to account for a Sanger output quality dependence on order of bases and base type in a sequence; based on facilitating statistical estimation, unmixing, deconvolution during computational post-processing; based on a number of base differences required to achieve a threshold abundance metric accuracy while minimizing amplification biases; etc.).

Additionally or alternatively, variation regions can include non-sequence variation regions, with functional, structural, evolutionary, and/or other suitable characteristics that are different from the characteristics of the one or more target molecules (e.g., of any suitable type, etc.). However, variation regions can be configured in any suitable manner, and synthetic target-associated molecules can include any suitable nucleotide sequence regions.

In some embodiments, the synthetic target-associated molecules include a target-matching region that matches a corresponding nucleotide sequence in a first region of the first infectious disease's RNA or DNA, and a target-variation region that is distinguishable from a second region of the first infectious disease's RNA or DNA, the target-variation region having a nucleotide sequence with an insertion or deletion as compared to a corresponding nucleotide sequence in the second region of the first infectious disease's RNA or DNA. For example, a target-matching region of the synthetic target-associated sequence that “corresponds to” the region of the infectious disease's DNA or RNA is a position (e.g., sequence position) that “matches” (e.g., has 100% sequence identity) to the position of the infectious disease's DNA or RNA. As used herein, the term “position” may refer to 1 or more nucleotide positions, 2 or more nucleotide positions, 3 or more nucleotide positions, 4 or more nucleotide positions, 5 or more nucleotide positions, 6 or more nucleotide positions, 7 or more nucleotide positions, 8 or more nucleotide positions, 9 or more nucleotide positions, or 10 or more nucleotide positions.

Additionally, a target-variation region of the synthetic target-associated sequence that “corresponds to” a second region of the first infectious disease's RNA or DNA is a position (e.g., sequence position) that is offset by the size of an insertion or deletion to the position of the infectious disease's DNA or RNA. As used herein, the term “position” may refer to 1 or more nucleotide positions, 2 or more nucleotide positions, 3 or more nucleotide positions, 4 or more nucleotide positions, 5 or more nucleotide positions, 6 or more nucleotide positions, 7 or more nucleotide positions, 8 or more nucleotide positions, 9 or more nucleotide positions, or 10 or more nucleotide positions.

In variations, synthetic target-associated molecules can include one or more sequencing regions (e.g., of sequencing molecules; etc.) configured to aid in sequencing operations (e.g., operation of sequencing systems; determination of sequencing outputs, such as of increased accuracy and/or of a form enabling quantitative comparison and/or quantification; etc.), determining abundance metrics, and/or any suitable portions of the method 100 (e.g., facilitating characterizations and/or facilitating treatment S16; etc.). In a variation, a target-associated molecule (e.g., a target-associated sequence of a target-associated molecule; etc.) can include (e.g., through addition of, etc.) one or more Sanger-associated sequence regions (e.g., configured to improve Sanger sequencing outputs, etc.) and/or any suitable sequencing regions, which can include any one or more of additional target-associated regions (e.g., with sequence similarity to additional target sequence regions of one or more target sequences, such as the same or different target sequences, of one or more biological targets, such as the same or different biological targets; etc.); sequence repeats (e.g., of any suitable regions of synthetic target-associated molecules, target molecules, reference-associated molecules, reference molecules, any suitable sequences, regions, and/or molecules described herein; etc.); and/or any suitable sequence regions (e.g., sequencing regions described herein in relation to being added to one or more molecules; etc.).

In a variation, the Sanger-associated sequence regions can include specific nucleotide sequences (e.g., of a predetermined length, with specifically selected nucleotides; etc.) preceding (and/or in any suitable positional relationship with) a sequence variation region and/or other suitable region of the target-associated molecule, which can facilitate repositioning of the sequence variation region to be at positions (e.g., at bases 100-500, at bases 200-500, during Sanger sequencing, and/or at any suitable positions) corresponding to improved Sanger sequencing chromatogram-related outputs. In a specific example, Sanger BigDye 1.1 chemistry can be applied for improved accuracy in relation to the beginning regions of a sequence (e.g., where LCR and/or RCA, can be omitted; etc.). In a specific example, Sanger BigDye 3.1 chemistry can be applied to enable longer sequencing reads, where a beginning sequence region (e.g., around 200 bp and/or other suitable size) can be used (e.g., inserted prior to the target sequence region and/or target-associated sequence region) for improved accuracy (e.g., such as through LCR and/or RCA, which can enable multiplexing). However, Sanger-associated sequence regions can be configured in any suitable manner.

Additionally or alternatively, sequencing molecules can include sequencing primers configured to facilitate processes by sequencing systems, adapter sequences, and/or other suitable components associated with any suitable sequencing systems. However, sequencing molecules can be configured in any suitable manner.

The synthetic target-associated molecules (and/or other suitable components described herein, such as reference-associated molecules, components of spike-in mixtures, etc.) can be of any suitable size (e.g., 100-500 base pairs, 200-500 base pairs, in length and including repeats of sequence regions, such as target-associated regions and/or variation regions; similar or different length as target molecules; 80-150 base pairs in length, including a variation region of two base pairs of shuffled base types; etc.). The set of synthetic target-associated molecules can include any number of synthetic target-associated molecules associated with any suitable number of targets (e.g., any number of target sequences associated with any number of loci, chromosomes, cancer biomarkers, target biomarkers, etc.), samples (e.g., concurrently synthesizing a batch of molecules for use with samples across multiple users, for user with multiple samples for a single user, to improve efficiency of the sample handling system; etc.), conditions (e.g., set of synthetic target-associated molecules associated with biological targets associated with different conditions; etc.), and/or other suitable aspects.

In variations, generating synthetic target-associated molecules can include generating different types of synthetic target-associated molecules (e.g., including different target-associated regions, different variation regions, different sequence molecules, etc.), such as sets of synthetic target-associated molecules (e.g., each set corresponding to a different type of synthetic target-associated molecules; etc.). Synthetic target-associated molecules can include sets of synthetic target-associated molecules (e.g., a plurality of different sets, etc.), each set including a different target-associated region associated with (e.g., with sequence similarity to; etc.) a different target sequence region (e.g., different target sequence regions of a same target sequence and/or biological target such as a chromosome; different target sequence regions of different target sequences and/or biological targets such as different genes; etc.), which can facilitate different pairs and/or sets of a target-associated region type (e.g., corresponding to a specific target-associated region sequence; etc.) and a target sequence region type (e.g., corresponding to a specific target sequence of a biological target; etc.), and/or different pair and/or sets of bases (e.g., where the bases of the pair and/or set can be from a target-associated sequence and a target sequence; etc.), such as to determine corresponding abundance metrics such as individual abundance ratios (e.g., corresponding to the different pairs; such as individual abundance ratios corresponding to different sets of bases, where the different sets of bases can correspond to different loci of a chromosome biological target; etc.), which can be used in determining an overall abundance metric with increased accuracy through, for example, averaging and/or performing any suitable combination operations with the individual abundance metrics.

In some embodiments, the synthetic target-associated molecules include a two or more, three or more, four or more, five or more, six or more, seven or more, eight or more, nine or more, or ten or more target-matching regions, where each target matching region matches a nucleotide sequence region of a different infectious disease genomic sequence. For example, a target-associated molecule can have a first target-matching region that matches a first region of an Influenza A virus's DNA or RNA, a second target-matching region that matches a first region of an Influenza B virus's DNA or RNA, a third target-matching region that matches a first region of a SARS-CoV-2 virus's DNA or RNA. This allows for multiplexed detection of more than one infectious disease in a single assay.

In some embodiments, the synthetic target-associated molecules are DNA molecules. In some embodiments, the synthetic target-associated molecules are RNA molecules. In some embodiments, the synthetic target-associated molecules are a mixture of DNA and RNA molecules. In some embodiments, the synthetic target-associated molecules is selected from one or more of: a nucleotide sequence, a peptide nucleic acid (PNA), a DNA/RNA hybrid, oligomers, oligonucleotide, polynucleic acid, a nucleotide sequence encoding a fusion molecule, a bridged nucleic acid, Multi-Functional Bridged Nucleic Acid (BNA), a nucleic acid analog, a locked nucleic acid, a cysteine-labeled DNA or RNA molecule, a PEG-labeled DNA or RNA molecule, a fluorescently labeled DNA or RNA molecule, DNA scaffold, RNA scaffold, and the like.

In certain embodiments, the synthetic target-associated molecules will also include two or more, three or more, four or more, five or more, six or more, seven or more, eight or more, nine or more, or ten or more target-variation regions, depending on the infectious disease being detected or the number of infectious diseases being tested for. For example, in the detection of SARs-CoV-2, two types of spike-in molecules may be used. The first spike-in molecule may be associated a wild type (WT) allele and include a single variation region. The second spike-in molecule may be associated with a mutated allele and may include two variation regions. In certain embodiments, each target-variation region is distinguishable from a second region of a different infectious disease's RNA or DNA. In certain embodiments, the synthetic target-associated molecules will also include 1-50, 30-50, 10-20, 1-10, 5-10, 1-3, 1-4, 1-5, 1-25, or 25-50 target-variation regions. Thus, in one aspect, the methods of the present disclosure can test for about 1-50 pathogens or infectious diseases. For example, a first target-variation region can have a nucleotide sequence with an insertion or deletion as compared to a corresponding nucleotide sequence in the second region of the Influenza A virus's RNA or DNA RNA, a second target-variation region can have a nucleotide sequence with an insertion or deletion as compared to a corresponding nucleotide sequence in the second region of the Influenza B virus's RNA or DNA RNA, a third target-variation region can have a nucleotide sequence with an insertion or deletion as compared to a corresponding nucleotide sequence in the second region of the SARS-CoV-2 virus's RNA or DNA RNA.

In some embodiments, the synthetic target-associated molecules include a two or more, three or more, four or more, five or more, six or more, seven or more, eight or more, nine or more, or ten or more target-variation regions. The location of the variation region may vary. In some embodiments, the variation region is located within the center of the amplicon sequence of the spike-in molecule, at an end of the amplicon (e.g. 5′ end or 3′ end), or the like.

In a specific example, different sets of synthetic target-associated molecules can be associated with different target sequences (and/or target sequence regions, etc.) across different loci. In a specific example, each set can be associated with a different locus for the same chromosome (e.g., a first, second, third, and fourth locus for a chromosome; etc.), where a sequence of a target-associated molecule of a given set can include a sequence region shared by the locus corresponding to the set, and can include a sequence variation region differing (e.g., by insertions, deletions, base substitutions, etc.) from the sequence for the locus.

Any number of sets of synthetic target-associated molecules and/or any number of types of synthetic target-associated molecules can be generated and/or associated with any suitable number of biological targets (e.g. biological targets associated with one or more infectious diseases). In an example, selecting different synthetic target-associated molecule sets can be based on sequencing parameters, accuracy requirements for a given condition and/or application (e.g., selecting a number of sets leading to a corresponding suitable number of individual abundance metrics to be used in achieving a target accuracy for diagnosing SARS-CoV-2), and/or can be selected based on any suitable criteria (e.g., parameter to be optimized). However, generating different sets of synthetic target-associated molecules can be performed in any suitable manner.

Generating synthetic target-associated molecules can include determining target sequences (e.g., target sequence regions of target sequences; any suitable regions of target sequences; etc.), which can function to select target sequences upon which the generation of synthetic target-associated molecules can be based. Determining target sequences can be based on any one or more of: infectious diseases (e.g., selecting target sequences identifying DNA or RNA sequences associated with an infectious disease; selecting target sequences identifying the infectious disease; etc.), sequencing parameters (e.g., selecting target sequences of a particular length, nucleotide sequence, and/or other parameter for optimizing chromatogram-related output quality from capillary electrophoresis; for generating chromatogram-related outputs suitable for statistical estimation analysis and/or other suitable; for reducing cost, improving accuracy, improving reproducibility, and/or for other suitable optimizations in relation to sequencing systems and/or operations, etc.); amplification parameters (e.g., selecting target sequences of a particular length, nucleotide sequence, and/or other parameter for optimizing amplification specificity, such as in relation to primer specificity for the target sequences in relation to polymerase chain reaction amplification; hybridization capture, ligation etc.), other sample processing parameters, and/or other suitable criteria. In an example, determining target sequences can include computationally searching a database (e.g., DNA database, genome database, gene expression database, phenotype database, RNA database, protein databases, etc.) to generate a target sequence candidate list; and filtering the target sequence candidate list based on criteria described herein, and/or any suitable criteria. In a specific example, determining targeting sequences can include extracting a target sequence candidate list (e.g., based on exome pull down; merge into chunks of a suitable number of base pairs; etc.); filtering out candidates including defined types of mutations and/or polymorphisms (e.g., filtering out candidates associated with common single nucleotide polymorphisms to obtain candidates with relative invariance across subjects of a population, etc.); identifying primers for the remaining candidates (e.g., with a Primer-BLAST for 80-150 bp amplicons); and determining candidate regions that are suitable for variation in generating a variation region of target-associated molecule (e.g., through scrambling bases at positions relative a forward primer and/or other region of the sequence, etc.). However, determining target sequences can be performed in any suitable manner.

Generating the synthetic target-associated molecules can include synthesizing the molecules through performing any one or more of: amplification (e.g., PCR amplification, such as with PCR primers including one or more hairpin sequences; etc., hybridization capture, ligation-based techniques), plasmid-based nucleic acid synthesis (e.g., including both synthetic target-associated molecules and reference-associated molecules respectively corresponding to different loci of a target DNA or RNA and a reference RNA or DNA; using plasmids including any suitable cut sites, origin of replication sites, multiple cloning sites, selectable markers, reporter markers, backbone, and/or other components; etc.), other artificial gene synthesis techniques, amplification techniques (e.g., polymerase chain reaction, rolling circle amplification, etc.), ligation techniques (e.g., Ligase Cycling Reaction, etc.), phosphoramidite approaches, post-synthetic processing, purification (e.g., using high-performance liquid chromatography or other chromatography approaches, desalting, washing, centrifuging, etc.), tagging techniques (e.g., molecular tagging techniques, fluorescent tagging techniques, particle labeling techniques, etc.), molecule cloning techniques, and/or any suitable sample processing technique.

In a variation, synthesizing synthetic target-associated molecules can include generating a target-associated sequence including a plurality of sequence regions associated with different targets (e.g., different viral genes, etc.). In a specific example, a type of target-associated molecule can be configured to reduce the number of required operations (e.g., a target-associated molecule type that facilitates generation of a chromatogram-related output informative of a plurality of targets and generated using a single Sanger sequencing run; or number of capillary electrophoresis runs or capillaries used etc.); however, target-associated molecule types can be synthesized to optimize for any suitable parameter. Additionally or alternatively, any suitable number of molecules and/or types of molecules associated with any number of targets can be generated at any suitable time and frequency.

5.1.4. Co-Amplification

Aspects of the present methods include co-amplifying the synthetic target-associated molecules and the target sample molecules to generate a co-amplified spike-in mixture (e.g., amplicon products).

Embodiments of the method 100 can include generating (e.g., facilitating generation of, etc.) one or more spike-in mixtures (e.g., based on processing the set of synthetic target-associated molecules with target sample molecules from one or more samples from a subject, etc.), which can function to amplify (e.g., under similar amplification parameters), perform pre-processing upon (e.g., sample preparation, lysis, bead-based processes, other purification and/or nucleic acid extraction techniques, etc.), modify (e.g., generate sequence repeats, combine sequences associated with different targets, etc.) and/or otherwise process the target-associated molecules, target molecules, and/or other suitable molecules (e.g., reference-associated molecules, reference molecules, etc.) into a form suitable for subsequent analysis (e.g., capillary electrophoresis with Sanger sequencing or fragment analysis, etc.) and abundance metric determination (e.g., based on outputs from the capillary electrophoresis methods etc.).

Generating one or more spike-in mixtures preferably includes combining synthetic target-associated molecules with target sample molecules (e.g., DNA or RNA nucleic acids including target sequence regions and/or target sequences, etc.) from the sample; and/or combining reference-associated molecules with reference molecules; and/or combing any suitable molecules. Combining can include one or more of: combining each of the molecules into a single mixture (e.g., including different subsets of synthetic target-associated molecules and corresponding subsets of target sample molecules; etc.); subsampling the sample (e.g., a preprocessed sample) into a plurality of mixtures, each designated for a different subset of synthetic target-associated molecules (e.g., corresponding to different target gene for a target gene, etc.); subsampling the sample into different mixtures for synthetic target-associated molecules and reference-associated molecules; and/or any other suitable approach to combining the molecules. In an example, target sample molecules and synthetic target-associated molecules (e.g., different pairs of types of target molecules and target-associated molecules; corresponding to different pairs of target-associated regions and target sequence regions; associated with a plurality of different targets; etc.) can be amplified in the same compartment (e.g., tube; etc.) (and/or any suitable number of compartments), such as through multiplex PCR and/or suitable amplification processes, which can facilitate conserving a precious sample; and the resulting amplification products can be subsequently subsampled into separate mixtures for subsequent capillary electrophoresis using targeting different target types (e.g., using a primer associated with an invariant region, such as a region of sequence similarity, shared by the target-associated region and target sequence region; etc.). In examples, subsampling and/or other sample modification operations can be performed in any suitable order.

Additionally or alternatively, separate samples (e.g., mixtures, solutions, etc.) can be generated for different types of molecule (e.g., without combining different types of molecules). For example, a first sample including synthetic target-associated molecules (e.g., without target sample molecules) can be generated, and a sample mixture including target sample molecules (e.g., without synthetic target-associated molecules) can be generated, where the first and second mixtures can be separately used in downstream processing (e.g., performing separate Sanger sequencing runs to generate separate chromatogram-related outputs such as separate chromatograms that can be used during statistical estimation, deconvolution, and/or other computational processing operations, such as for determining abundance metrics, etc.). However, any suitable number of samples including any suitable separate or combination of types of molecules can be generated and/or processed.

Combining molecules includes using a known abundance of synthetic target-associated molecules, but an unknown abundance of target sample molecules can alternatively be used (e.g., where results from preceding sequencing runs with the unknown abundance can be used to inform results from subsequent sequencing runs, etc.). Further, combining molecules preferably includes using the same or substantially similar abundances across different subsets of synthetic target-associated molecules (e.g., associated with different loci), and/or same or similar abundances relative to reference-associated molecules. Additionally or alternatively, any suitable abundances for different molecule types can be used.

In a variation, combining molecules can include modifying (e.g., during pre-processing) abundances of the synthetic target-associated molecules, the reference-associated molecules, and/or other suitable components. For example, modifying abundances of molecules can include measuring initial abundances of the molecules (e.g., abundance of the synthetic target-associated molecules); and modifying the abundances (e.g., through dilution, amplification, etc.) based on expected abundances of target molecules (e.g., expected count for endogenous target molecules in the sample, etc.). In a variation, generating spike-in mixtures can omit modification (e.g., during pre-processing) of abundances. However, combining molecules can be performed in any suitable manner.

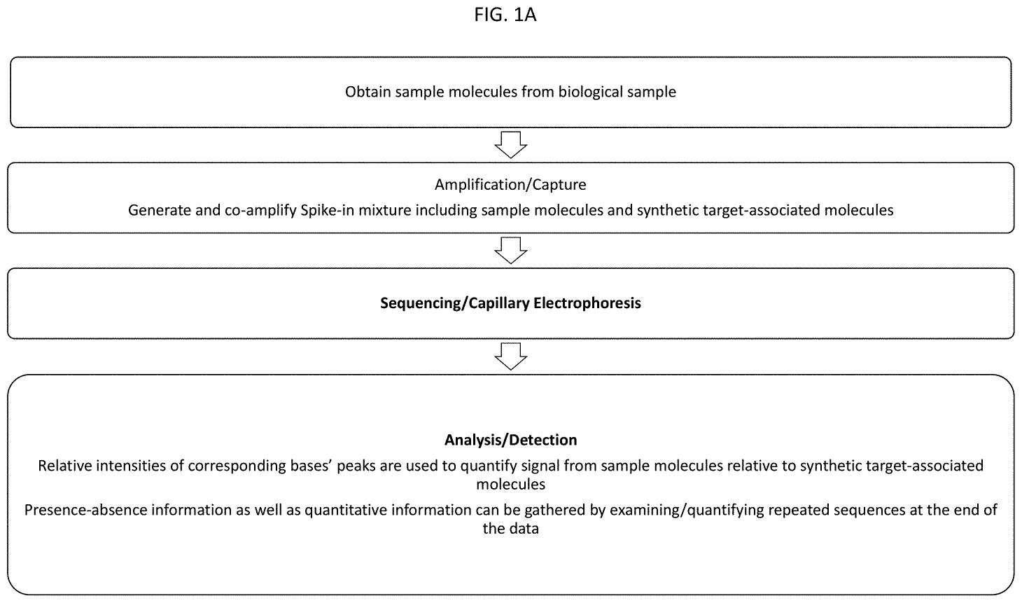

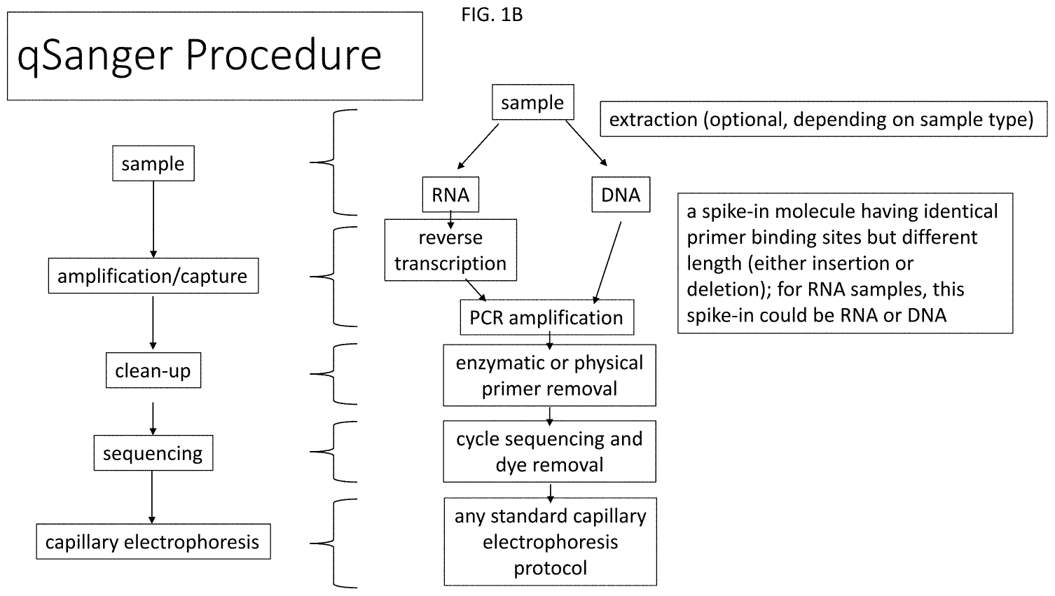

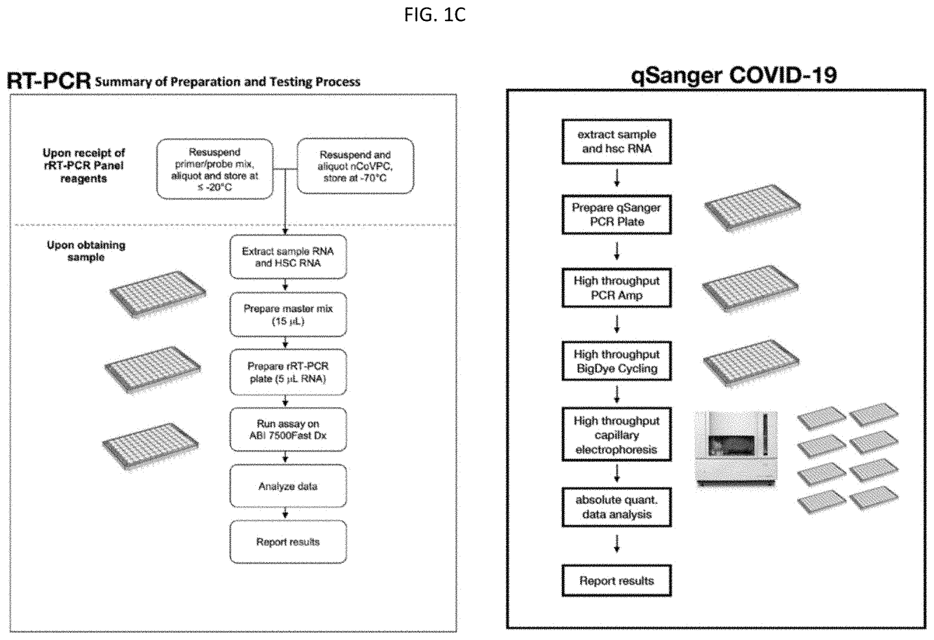

In some embodiments, generating the spike-in mixture includes amplifying the target-associated molecules with the target molecules. Amplification can include performing any one or more of: polymerase chain reaction-based techniques (e.g., solid-phase PCR, RT-PCR, qPCR, multiplex PCR, touchdown PCR, nanoPCR, nested PCR, hot start PCR, etc.), helicase-dependent amplification (HDA), loop mediated isothermal amplification (LAMP), self-sustained sequence replication (3 SR), nucleic acid sequence based amplification (NASBA), strand displacement amplification (SDA), rolling circle amplification (RCA), ligase chain reaction, ligase cycling reaction (LCR), and/or any other suitable amplification techniques and/or associated protocols (e.g., protocols for minimizing amplification bottlenecking). In an example, as shown in FIG. 1C, generating a spike-in mixture can include performing a plurality of PCR rounds (e.g., any number of PCR rounds) to co-amplify the target-associated molecules with the target molecules (e.g., using sets of primers targeting a sequence shared by both the synthetic target-associated molecules and the target molecules; using different sets of primers corresponding to different primer types and sequences, where one or more of the sets of primers can include one or more hairpin sequences, such as for facilitating addition of sequence repeats; etc.).

In certain embodiments, generating a spike-in mixture where samples including target-associated molecules are independently prepared and sequenced from samples including target molecules; where samples including reference-associated molecules are independently prepared and sequenced from samples including reference molecules; etc., can include adding one or more sequence regions to one or more molecules (e.g., one or more regions and/or sequences of one or more molecules; to target-associated molecules, to target molecules, to reference-associated molecules, to reference molecules, etc.).

Adding sequence regions can include one or more of: generating sequence repeats (e.g., generating a modified sequence including repeats of a target-associated sequence and/or target sequence; etc.); adding sequence regions identifying different targets (e.g., different loci of a chromosome identified by the original target; loci of different chromosomes; sequence regions associated with different conditions; etc.); and/or adding any suitable nucleotide sequences, e.g., for fluorescently labeling a target sample region or synthetic target associated molecule region. For example, the method can include adding at least one sequence region to at least one of the set of synthetic target-associated molecules and the target sample molecules, where the at least one sequence region includes at least one of (a) a second target-associated region with sequence similarity to a second target sequence region (e.g., where the set of target-molecules includes a first target-associated region with sequence similarity to a first target sequence region of a target sequence of a biological target; etc.), and (b) at least one sequence repeat of at least one of a region of the target-associated sequence and a region of the target sequence.

Adding sequence regions can function to: facilitate improved output quality from sequencing systems (e.g., quality of chromatogram results), such as through adding sequence regions positionally preceding variation regions upon which abundance metric extraction will be based (e.g., where the added sequence regions can enable repositioning of the variation regions to be at positions corresponding to improved sequencing outputs; etc.); facilitate determination of additional individual abundance metrics for the added sequence regions (e.g., by analyzing sequence repeats of the variation region in relation to corresponding target bases; etc.), which can be used in calculating an overall abundance metric of improved accuracy; facilitate reduction in number and/or cost of required sequencing operations (e.g., fewer capillary electrophoresis runs; etc.) to analyze a plurality of targets (e.g., across different loci, chromosomes, genes, etc.), such as through ligating different sequences associated with the different targets.

In certain embodiments, adding one or more sequence regions (e.g., sequence repeats; etc.) can be based on one or more hairpin sequences (e.g., of primers, such as used in PCR amplification; etc.), such as where amplification with PCR primers including the one or more hairpin sequences can enable a plurality of nucleotide extension instances (e.g., through self-priming) for adding sequence repeats that can run through capillary electrophoresis. For example, adding one or more sequence repeats (e.g., to any suitable molecules; etc.) can include co-amplifying (and/or separately amplifying), with one or more sets of primers including one or more hairpin sequences or universal tail sequences, the set of target-associated molecules and nucleic acid molecules from the sample (e.g., biological sample; etc.), where the nucleic acid molecules include the target sequence region. In an example, primers (e.g., PCR primers) including a hairpin sequence can include one or more portions (e.g., sequence portions, structural portions; etc.) of a forward/reverse primer sequence shown in FIGS. 2B and 23.

However, target-associated sequences, target sequences, and/or other suitable sequences can be modified in any suitable manner (e.g., deleting regions, modifying nucleotides at specific positions, etc.) using any suitable sample processing operations. However, generating spike-in mixtures can be performed in any suitable manner.

5.1.4.1 Primers

Aspects of the present methods include co-amplifying the spike-in mixture comprising the synthetic target-associated molecules and the target sample molecules.

In some embodiments, co-amplifying the spike-in mixture with a plurality of primer sequences. A plurality includes one or more sets of primer sequences comprising nucleotide sequences that are complementary to the target matching region of the synthetic target-associated molecules and are complementary to the first region of the infectious disease's RNA or DNA.

For example, FIG. 2 and FIG. 13 show a plurality of primer sequences comprising: forward and reverse primer sequences (“primer sequences” in bold) that are complementary to a target matching region of the synthetic target-associated molecules (e.g., bold and underlined sequence of “spike in sequence”) and that are complementary to the first region of the infectious disease's RNA or RNA (see e.g., bold and underlined region of the “Genomic sequence” for each infectious disease).

In some embodiments, the plurality of primer sequences comprises a first set of primer sequences, a second set of primer sequences, and a third set of primer sequences, each set of primer sequences having sequence complementary to a different infectious disease's DNA or RNA region. In certain embodiments, the plurality of primer sequences comprise multiple sets of primer sequences, each set corresponding to a different biological target (e.g., one or more sets, two or more sets, three or more sets, four or more sets, five or more sets, six or more sets, seven or more sets, eight or more sets, nine or more sets, or ten or more sets).

As a non-limiting example, the plurality of primers comprise a first set of primer sequences, including forward and reverse primers that are complementary to the first region of a nucleotide sequence of a first infectious disease's DNA or RNA, a second set of primer sequences, including forward and reverse primers that are complementary to the first region of a nucleotide sequence of a second infectious disease's DNA or RNA, and a third set of primer sequences, including forward and reverse primers that are complementary to the first region of a nucleotide sequence of a third infectious disease's DNA or RNA.

In some embodiments, primers of the present methods are configured to have complementarity specificity to target molecules of interest. Thus, the target sample molecule comprises the genomic DNA or RNA regions associated with an infectious disease's DNA or RNA, then the primers will have complementarity to the target region of the target sample sequence. Primers can also help facilitate amplification of the spike-in mixture, creating a co-amplified spike-in mixture (e.g., amplicon products of the spike in mixture molecules).

5.1.4.2 Fluorescently Labeled Primers

In some embodiments, the plurality of primers comprises one or more sets of fluorescently labeled primers.

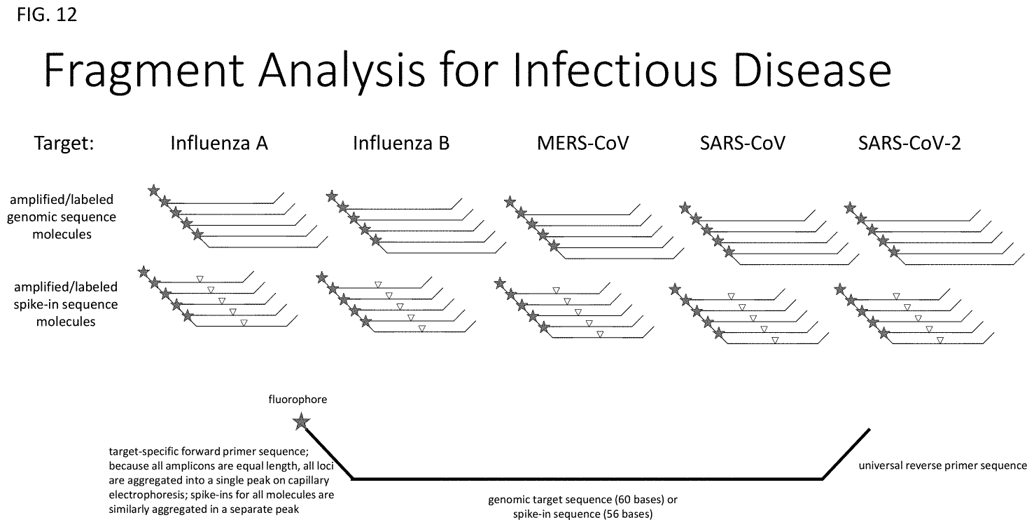

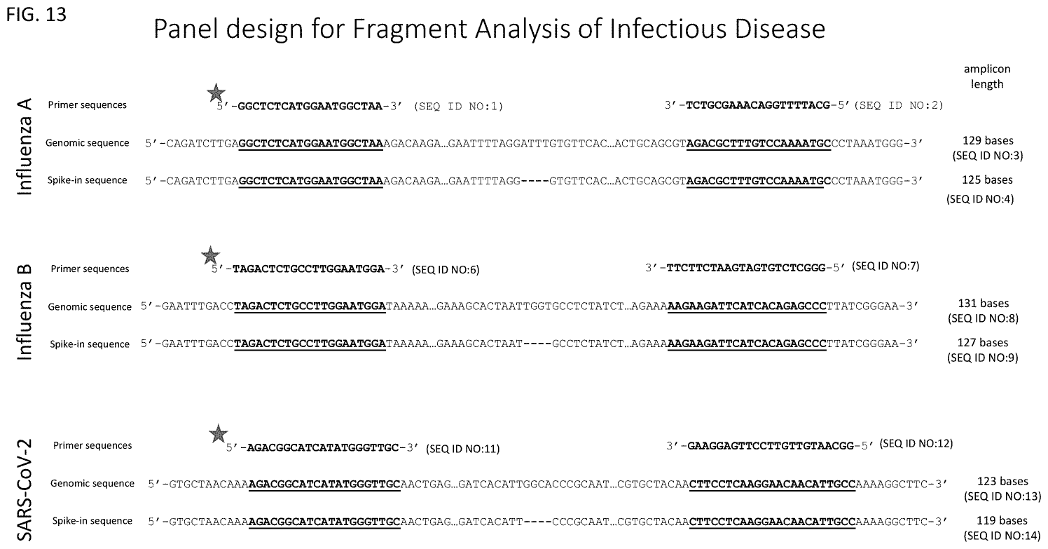

As shown in FIG. 12-13, fluorescently labeled primers can be used to selectively amplify capture molecules for each infectious disease in a multiplexed manner. In some embodiments, each genomic target sequence of interest of the infectious disease can have the same fluorophore and then be analyzed on different capillaries during capillary electrophoresis, or multiple sized labels can be used in the same color to co-label or multiple sizes can be reused to resample the same molecules.

In certain embodiments, the plurality of primer sequences can further include universal primer sequences (e.g. universal tailed sequences) to increase the length of one or more target molecules and/or for facilitating fluorescent labeling. For example, tails primer sequences can be added to aggregate e.g., specific synthetic target-associated molecules, specific target sample molecules, for labeling. Alternatively, capture mechanisms can also be pre-labeled (e.g., using fluorescently labeled primers) and therefore no additional labeling step would be required.

Additionally, amplified spike-in molecules can be resampled during capillary electrophoresis by introducing different sized labeling primers. Thus, in some embodiments, the plurality of primer sequences comprises sets of primers with various nucleotide lengths. For example, fluorescently (FAM) labeled primers specifically capture each genomic sequence but can generate different length amplicons for e.g., Influenza A, Influenza B, SARS-CoV-2 (129 bases, 131 bases, and 123 bases respectively); and spike-in amplicons are correspondingly 4 bases shorter (125 bases, 127 bases, and 119 bases). Similarly for FIG. 2D, non-fluorescent primers specifically capture each genomic sequence but can generate different length amplicons for e.g., Influenza A, Influenza B, SARS-CoV-2 (129 bases, 131 bases, and 123 bases respectively); and spike-in amplicons are correspondingly 4 bases shorter (125 bases, 127 bases, and 119 bases).

In certain embodiments, the plurality of primers comprises hairpin primer sequences.

5.1.4.3 Tail Primers

In some embodiments, the plurality of primers further include tail sequencing primers. Tail sequencing primers can be of any length of interest.

In certain embodiments, the tail sequencing primers comprise universal tail primers.

In some embodiments, tail sequence primers can be used to change the length of the co-amplified spike in mixtures. For example, tail sequences can be used to add additional nucleotides to the amplified synthetic target-associated molecules or the target sample molecule. Inserted nucleotides can include 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more nucleotides. The nucleotides can be added to the 5′ end or the 3′ end of the synthetic target-associated molecules and/or the target sample molecules.

In certain embodiments, the tail sequence primers can facilitate fluorescent labeling of the fluorescently labeled primers.

In some embodiments, tail sequence primers can modify the length of the synthetic target-associated molecules or amplicon products and the sample molecules or amplicon products in a secondary amplification step.

5.1.4.4 Sanger Primers

In some embodiments where sanger sequencing is performed during the capillary electrophoresis process, the method comprises sanger primers (see e.g, FIG. 2D).

In some embodiments, the sanger sequencing primers can add and amplify sanger nucleotide sequences to the synthetic-target associated molecules and the target sample molecule to facilitate sanger sequencing.

For example, in some embodiments, the synthetic target-associated molecules can include one or more sequencing regions (e.g., of sequencing molecules; etc.) configured to aid in sequencing operations (e.g., operation of sequencing systems; determination of sequencing outputs, such as of increased accuracy and/or of a form enabling quantitative comparison and/or quantification; etc.), determining abundance metrics, and/or any suitable portions of the method (e.g., facilitating characterizations and/or facilitating treatment; etc.). In certain embodiments, a synthetic target-associated molecule (e.g., a target-associated sequence of a target-associated molecule; etc.) can include (e.g., through addition of, etc.) one or more Sanger-associated sequence regions (e.g., configured to improve Sanger sequencing outputs, etc.) and/or any suitable sequencing regions, which can include any one or more of additional target-associated regions (e.g., with sequence similarity to additional target sequence regions of one or more target sequences, such as the same or different target sequences, of one or more biological targets, such as the same or different biological targets; etc.); sequence repeats (e.g., of any suitable regions of target-associated molecules, target molecules, reference-associated molecules, reference molecules, any suitable sequences, regions, and/or molecules described herein; etc.); and/or any suitable sequence regions (e.g., sequencing regions described herein in relation to being added to one or more molecules; etc.).

5.1.5. Capillary Electrophoresis

Aspects of the present methods include performing capillary electrophoresis on the co-amplified spike-in mixture to generate a chromatogram-related output comprising a plurality of chromatogram intensities.

In some embodiments, the intensities include a peak intensity associated with synthetic target-associated molecules and sample molecules of the subject.

In some embodiments, the intensities include an intensity associated with: the target-matching region of the synthetic target-associated molecules; the target-variation region of the synthetic target-associated molecules; and a region of the sample molecules of the subject that corresponds to the target-variation region of the synthetic target-associated molecules.

In some embodiments, the plurality of chromatogram intensities include an intensity associated with: amplicon products having the target-associated nucleotide length; and amplicon products having the sample nucleotide length, wherein the synthetic target-associated amplicon products have a target-associated nucleotide length that is different by a predetermined amount than a sample nucleotide length of the sample amplicon products.

Capillary electrophoresis refers to capillary electrophoresis methods containing at least 1 capillary, at least 2 capillaries, at least 3 capillaries, at least 4 capillaries, at least 5 capillaries, at least 6 capillaries, at least 7 capillaries, at least 8 capillaries, at least 9 capillaries, or at least 10 capillaries. The methods of the present disclosure are particularly well-suited for use in capillary electrophoresis systems.

In capillary electrophoresis, a buffer-filled capillary is suspended between two reservoirs filled with buffer. An electric field is applied across the two ends of the capillary. The electrical potential that generates the electric field is in the range of kilovolts. Samples containing one or more components or species are typically introduced at the high potential end and under the influence of the electrical field. Alternatively, the sample is injected using pressure or vacuum. The same sample can be introduced into many capillaries, or a different sample can be introduced into each capillary. In some embodiments, an array of capillaries is held in a guide and the intake ends of the capillaries are dipped into vials that contain samples. After the samples are taken in by the capillaries, the ends of the capillaries are removed from the sample vials and submerged in a buffer which can be in a common container or in separate vials. The samples migrate toward the low potential end. During the migration, components of the sample are electrophoretically separated. After separation, the components are detected by a detector. Detection may be effected while the samples are still in the capillaries or after they have exited the capillaries.

The channel length for capillary electrophoresis is selected such that it is effective for achieving proper separation of species. Generally, the longer the channel, the greater the time a sample will take in migrating through the capillary. Thus, the species may be separated from one another with greater distances. However, longer channels contribute to the band broadening and lead to excessive separation time. In some embodiments, for capillary electrophoresis, the capillaries are about 10 cm to about 5 meters long, or about 20 cm to about 200 cm long. In capillary gel electrophoresis, where typically a polymer separation matrix is used, in some embodiments, the channel length is about 10 cm to about 100 cm long.

The internal diameter (i.e., bore size) of the capillaries is not critical, although small bore capillaries are more useful in highly multiplexed applications. In some embodiments, to a wide range of capillary sizes can be used in the present methods. In general, capillaries can range from about 5-300 micrometers in internal diameter, with about 20-100 micrometers preferred. The length of the capillary can generally range from about 100-3000 mm, or about 300-1000 mm.

The use of machined channels instead of capillaries has recently been reported (R. A. Mathies et al., Abstract #133, DOE Human Genome Workshop IV, Santa Fe, N. Mex., Nov. 13-17, 1994; J. Balch et al., Abstract #134, DOE Human Genome Workshop IV, Santa Fe, N. Mex., Nov. 13-17, 1994). With conventional technology, however, multiple capillaries are still the more developed format for multiplexed CE runs. However, technologies developed for capillaries, such as those disclosed herein, are readily transferable to machined channels when that technology becomes more developed.

A suitable capillary is constructed of material that is sturdy and durable so that it can maintain its physical integrity through repeated use under normal conditions for capillary electrophoresis. It is typically constructed of nonconductive material so that high voltages can be applied across the capillary without generating excessive heat. Inorganic materials such as quartz, glass, fused silica, and organic materials such as polytetrafluoroethylene, fluorinated ethylene/propylene polymers, polyfluoroethylene, aramide, nylon (i.e., polyamide), polyvinyl chloride, polyvinyl fluoride, polystyrene, polyethylene and the like can be advantageously used to make capillaries.

Where excitation and/or detection are effected through the capillary wall, a particularly advantageous capillary is one that is constructed of transparent material, as described in more detail below. A transparent capillary that exhibits substantially no fluorescence, i.e., that exhibits fluorescence lower than background level, when exposed to the light used to irradiate a target species is especially useful in cases where excitation is effected through the capillary wall. Such a capillary is available from Polymicro Technologies (Phoenix, Ariz.). Alternatively, a transparent, non-fluorescing portion can be formed in the wall of an otherwise nontransparent or fluorescing capillary so as to enable excitation and/or detection to be carried out through the capillary wall. For example, fused silica capillaries are generally supplied with a polyimide coating on the outer capillary surface to enhance its resistance to breakage. This coating is known to emit a broad fluorescence when exposed to wavelengths of light under 600 nm. If a through-the-wall excitation scheme is used without first removing this coating, the fluorescence background can mask a weak analyte signal. Thus, a portion of the fluorescing polymer coating can be removed by any convenient method, for example, by boiling in sulfuric acid, by oxidation using a heated probe such as an electrified wire, or by scraping with a knife. In a capillary of approximately 0.1 mm inner diameter or less, a useful transparent portion is about 0.01 mm to about 1.0 mm in width.

In electrophoresis, the separation buffer is typically selected so that it aids in the solubilization or suspension of the species that are present in the sample. Typically the liquid is an electrolyte which contains both anionic and cationic species. Preferably the electrolyte contains about 0.005-10 moles per liter of ionic species, more preferably about 0.01-0.5 mole per liter of ionic species. Examples of an electrolyte for a typical electrophoresis system include mixtures of water with organic solvents and salts. Representative materials that can be mixed with water to produce appropriate electrolytes includes inorganic salts such as phosphates, bicarbonates and borates; organic acids such as acetic acids, propionic acids, citric acids, chloroacetic acids and their corresponding salts and the like; alkyl amines such as methyl amines; alcohols such as ethanol, methanol, and propanol; polyols such as alkane diols; nitrogen containing solvents such as acetonitrile, pyridine, and the like; ketones such as acetone and methyl ethyl ketone; and alkyl amides such as dimethyl formamide, N-methyl and N-ethyl formamide, and the like. The above ionic and electrolyte species are given for illustrative purposes only. A researcher skilled in the art is able to formulate electrolytes from the above-mentioned species and optionally species such an amino acids, salts, alkalis, etc., to produce suitable support electrolytes for using capillary electrophoresis systems.

The voltage used for electrophoretic separations is not critical to the methods, and may vary widely. Typical voltages are about 500 V-30,000 V, or about 1,000-20,000 V.

Electrophoretic separation can be conducted with or without using a molecular matrix (also referred to herein as a sieving matrix or medium as well as a separation matrix or medium) to effect separation. Where no matrix is used, the technique is commonly termed capillary zone electrophoresis (CZE). Where a matrix is used, the technique is commonly termed capillary gel electrophoresis (CGE). In some embodiments, the separation matrix that can be used in CGE is a linear polymer solution, such as a poly(ethyleneoxide) solution. However, other separation matrices commonly used in capillary electrophoresis, such as cross-linked polyacrylamide, can also be used in various aspects of the methods. Suitable matrices can be in the form of liquid, gel, or granules.

The present methods may be used for the separation, detection and measurement of the amplified spike-in molecules of the present methods.

In some embodiments, nucleic acids and oligonucleotides such as RNA, DNA, their fragments and combinations, chromosomes, genes, sequence regions, as well as fragments and combinations thereof can be detected using capillary electrophoresis. Capillary electrophoresis can be used for DNA or RNA diagnostics, such as DNA or RNA sequencing, DNA or RNA fragment analysis, and DNA or RNA fingerprinting. Sequence variations as small as one base or base pair difference between a sample and a control can be detected.

5.1.5.1 Sanger Sequencing

In some embodiments of the present methods, performing capillary electrophoresis on the co-amplified spike-in mixture comprises sanger sequencing the co-amplified spike-in mixture.

Following sanger sequencing, a chromatogram-related output is generated.

In some embodiments when using a sanger sequencing mode during capillary electrophoresis, one or more peaks are generated by the sequence of the sample molecule and one or more peaks are generated by the sequence of the synthetic target-associated molecule, which are used to detect the presence or absence of an infectious disease.

In some embodiments, the chromatogram-related output comprises peak data associated with a plurality of chromatogram intensities, the intensities including one or more peak intensities associated with synthetic target-associated molecules and sample molecules of the subject. In some embodiments, the method includes determining the presence or absence of at least one infectious disease by comparing the chromatogram intensities associated with the synthetic target-associated molecules and the sample molecules of the subject.

In some embodiments, the method includes determining the presence or absence of the infectious disease by comparing the peak intensity position associated with the synthetic target-associated molecules and the peak intensity position of the sample molecules of the subject, wherein the peak intensity position of the synthetic target-associated molecules is offset as compared to the peak intensity position of the sample molecules.

In certain embodiments, the peak intensity position of the sample molecules is offset by a distance away (e.g., shifted left or right, a distance away by a number of nucleotides such as 1 or more nucleotides, 2 or more nucleotides, 3 or more nucleotides, 4 or more nucleotides, 5 or more nucleotides, 6 or more nucleotides, 7 or more nucleotides, 8 or more nucleotides, 9 or more nucleotides, 10 or more nucleotides away, 15 or more nucleotides away, 20 or more nucleotides away, 25 or more nucleotides away, 30 or more nucleotides away, and the like) from the peak intensity of the synthetic target-associated molecule.

In some embodiments, the peak intensity position of the sample molecule is offset by one or more nucleotides associated with the insertion or deletion of the target-variation region of the synthetic target-associated molecules.

In some embodiments, the peak intensity of the region of the sample molecules that corresponds to the target-variation region of the synthetic target-associated molecules includes a peak intensity position that is offset as compared to the peak intensity position of the target-variation region, where the peak intensity position is offset by one or more nucleotides associated with the insertion or deletion of the target-variation region.

In some embodiments, the method further comprises calculating the ratio of peak intensities of the region of the sample molecules that corresponds to the target-variation region of the synthetic target-associated molecules, to the peak intensities of the target variation region of the synthetic target-associated molecules.

In some embodiments, the method further includes: determining a first set of target-associated abundance metrics, wherein each target-associated abundance metric corresponds to a different pair of a base between a base of a nucleotide sequence of the synthetic target-associated molecules and a base of a nucleotide sequence of the sample molecules.

In certain embodiments, determining the first set of synthetic target-associated abundance metrics comprises, for each of the different pairs: determining a peak intensity metric for the base of the synthetic target-associated nucleotide sequence of the pair, based on the chromatogram-related output; determining a peak intensity metric for the base of the nucleotide sequence of the sample molecule of the pair, based on the chromatogram-related output; determining a target-associated abundance metric of the first set of target-associated abundance metrics, based on the peak intensity metric for the base of the synthetic target-associated nucleotide sequence and the peak intensity metric for the base of the nucleotide sequence of the sample; and determining an overall target-associated abundance metric based on the first set of target-associated abundance metrics; and

In some embodiments, the method further comprises determining the presence or absence of the infectious disease based on a comparison between the overall synthetic target-associated abundance metric and a reference-associated overall abundance metric describing abundance of a biological reference relative reference-associated molecules.

In certain embodiments, the method does not consist of RNA extraction of the from the sample molecules of the subject.

In some embodiments, the chromatogram-related output comprises alignment positions corresponding to the chromatogram intensities.

In certain embodiments, the chromatogram intensities comprise first peaks associated with: the target-matching region of the synthetic target-associated molecules; the target-variation region of the synthetic target-associated molecules; the region of the sample molecules of the subject that corresponds to the target-variation region of the synthetic target-associated molecules.

In certain embodiments, for each of the different pairs, the base of the nucleotide sequence of the synthetic target-associated molecule corresponds to a first alignment position that is different from a second alignment position corresponding to the base of the nucleotide sequence of the sample molecule, and wherein the alignment positions of the chromatogram-related output comprise the first and the second alignment positions.

In a non-limiting example, the amplification products of the co-amplified mixture are purified and Sanger sequenced by automated capillary electrophoresis. Synthetic DNA in RT-PCR master mix prior to PCR amplification can serve as an internal control that enables specimens to be readily identified as either positive or negative for an infectious disease such as COVID-19 (FIG. 2B-C). Quantitative analysis of the Sanger sequence chromatogram gives qSanger an extremely high sensitivity and specificity for all positive results with a limit of detection of 10-20 genome copy equivalents (GCE), equivalent to gold-standard qPCR methods. Furthermore, the presence of a spike-in as an intra-sample control in the qSanger assay allows for easy interpretation of results and determination of sources of error (e.g. extraction or amplification or sequencing failure), and also allows population-level analyses such as mutational analysis and contact tracing. In addition, the ratio of the amplitudes of corresponding bases between the endogenous and spike-in sequences at offset positions reflects the ratio of the molecular abundances of the two sequences. Computationally combining the amplitude ratios of multiple corresponding bases can then be used to estimate the viral load over a 400-fold dynamic range with Poisson-limited coefficient of variation.

In some embodiments, qsanger sequencing detects as low as 10-20 viral genome copy equivalents, even when VTM is added directly into the reaction mix without RNA extraction. In certain embodiments, the methods of the present disclosure does not consist of RNA extraction of the sample molecule. Thus, qSanger comprises an end-point PCR reaction with an internal spike-in control, it is more robust to inhibitors that can exist in VTM, and failures in amplification result in undetermined results that require a repeat reaction, as opposed to false negatives that would be obtained by qPCR. In some embodiments, the sequencing information obtained from sanger sequencing (e.g., qsanger) can be used to distinguish similar viruses and rule out false-positives due to non-specific amplifications.

In some embodiments, longer sequences of the synthetic target-associated molecules can be designed to capture a wide range of mutations in the qSanger reaction, as an infectious virus mutates and creates sub-strains with different clinical implications.

In some embodiments, absolute measurements of viral load are obtained in qSanger due to the known molecular count of the spike-in synthetic target-associated molecule.

In some embodiments, the method of the present disclosure can include adding at least one sequence repeat (e.g., for facilitating multiple-pass sequencing, such as sequencing sequences a plurality of times, such as in the same or different sequencing runs, such as for increasing sequencing output data, such that the sequencing output data and/or or associated abundance metrics can be averaged and/or otherwise combined, such as to reduce noise; etc.) to one or more target-associated molecules (e.g., one or more regions of a target-associated sequence of the target-associated molecules; etc.) and/or one or more target molecules (e.g., one or more regions of a target sequence of the target molecules; etc.), such as where the first set of peaks of the at least one chromatogram-related output (e.g., chromatogram, peak intensities, other peak data, etc.) correspond to a first sequencing (e.g., from a Sanger sequencing operation; etc.) for the target-associated region, the target sequence region of the biological target, the target variation region, and the sequence region of the biological target, where the at least one chromatogram-related output includes a second set of peaks corresponding to a second sequencing (e.g., from the same Sanger sequencing operation, etc.) for the target-associated region, the target sequence region of the biological target, the target variation region, and the sequence region of the biological target, and where determining a set of target-associated abundance ratios can be based on the first set of peaks and the second set of peaks (e.g., based on individual abundance ratios of peak intensities, from the first and the second set of peaks, for pairs of bases of the target sequence and the target-associated sequence; etc.).

In a non-limiting example, if a hairpin is used only at one end of the sequence (e.g., of a PCR primer sequence), a two-pass Sanger sequence is obtained (e.g., a chromatogram-related outputs including peak results for two passes of the sequence). In an example, a significant contribution of two-pass Sanger sequence is that the second sequence (e.g., second set of peak data and/or suitable chromatogram-related outputs for the sequence, etc.) can be more informative and cleaner in chromatogram content than the first-pass sequence (e.g., first set of peak data and/or suitable chromatogram-related outputs for the sequence, etc.) because of the decreased effect of primer-dimers at this increased length and because of the improved quality at longer lengths with Big Dye 3.1 chemistry. In examples, if a hairpin is used at both-ends, a plurality (e.g., many, multiple; etc.) rather than two (e.g., a plurality greater than two), Sanger chromatograms for the same sequence would be obtained; while this can significantly decrease any noise associated with abundance measurement due to averaging, it may not similarly decrease the effects of primer-dimers.

In certain embodiments, hairpin sequences (e.g., of primers, etc.) can be configured for, generated for, used for, and/or otherwise processed without target-associated molecules and reference-associated molecules. For example, one or more sequence repeats (e.g., generated through amplification with PCR primers including one or more hairpin sequences) can be added to target molecules, reference molecules, and/or suitable molecules for enabling Sanger sequencing (and/or suitable sequencing technologies) of a particular sequence for a plurality of instances (e.g., multiple pass sequencing to enable multiple sets of data to be generated for the same sequence in a single sequencing run; etc.). In an example, the ratio of a major allele peak (e.g., peak intensity metric) (and/or suitable chromatogram-related output; etc.) to a minor allele peak (e.g., peak intensity metric) (and/or suitable chromatogram-related output; etc.) can be determined a plurality of times based on outputs from Sanger sequencing the sequence repeats (e.g., generated from use of hairpin sequences; etc.) to determine an overall abundance ratio. Additionally or alternatively, adding one or more sequence regions can be performed without processing of target-associated molecules and/or reference-associated molecules, such as a where adding the one or more sequence regions can independently improve (e.g., accuracy of; reduction of bias regarding; reduction of noise regarding; etc.) chromatogram-related outputs, abundance metrics, characterizations, and/or treatments.

However, hairpin sequences can be configured in any suitable manner, and adding one or more sequence regions based on hairpin sequences can be performed in any suitable manner.

In some embodiments, adding sequence regions includes adding sequence regions to a mixture of amplicons including target molecule-based amplicons (e.g., amplicons generated from endogenous target molecules) and target-associated molecule-based amplicons (e.g., amplicons generated from spike-in target-associated molecules). Alternatively, adding sequence regions can be performed on the target-associated molecules separately from the target molecules. Additionally, or alternatively sequence regions can be initially generated (e.g., during generation of target-associated molecules, reference-associated molecules, etc.), such as to be part of the initial target-associated sequence and/or reference-associated sequence. Adding sequence regions can include performing any of the sample processing operations described herein, and/or other suitable operations. However, sequence regions can be configured in any suitable manner, and adding sequence regions can be performed in any suitable manner.

5.1.5.1.1 Performing a Sequencing Operation.

In certain embodiments, when sanger sequencing is performed, the method includes performing one or more sequencing operations (e.g., on the one or more spike-in mixtures, etc.), which can function to sequence one or more components (e.g., one or more spike-in mixtures; etc.) and/or generate one or more sequencing outputs. In some embodiments, performing sequencing operations preferably includes performing Sanger sequencing (e.g., on a spike-in mixture, on target molecules separately, on target-associated molecules separately, etc.). Sanger sequencing includes chain-termination approaches and/or any suitable operations related to Sanger sequencing (e.g., using labeled dideoxynucleotides and DNA polymerase, such as during in vitro DNA replication; generating a set of nucleic acid fragments covering base positions for bases of target-associated sequences, target sequences, reference-associated molecule sequences, reference sequences, any suitable sequences; performing analysis of the nucleic acid fragments, such as through capillary gel electrophoresis, laser detection of labelled bases; performing any suitable Sanger sequencing-related operations such as dye-terminator sequencing, automation and/or sample preparation associated with Sanger sequencing, microfluidic Sanger sequencing, computational processes to determine sequencing outputs; etc.). However, Sanger sequencing can be performed in any suitable manner.

In some embodiments, performing sequencing operations includes sequencing one or more co-amplified spike-in mixtures (e.g., a spike-in mixture including co-amplified target-associated molecules and nucleic acids including an associated target sequence region; etc.), but can additionally or alternatively sequence any suitable components (e.g., separately sequencing target-associated molecules from a first sample and target molecules from a second sample; spike-in mixtures; samples from users; samples including reference-associated molecules and/or reference molecules; etc.) with any number of sequencing operations (e.g., any number of Sanger sequencing runs, etc.).

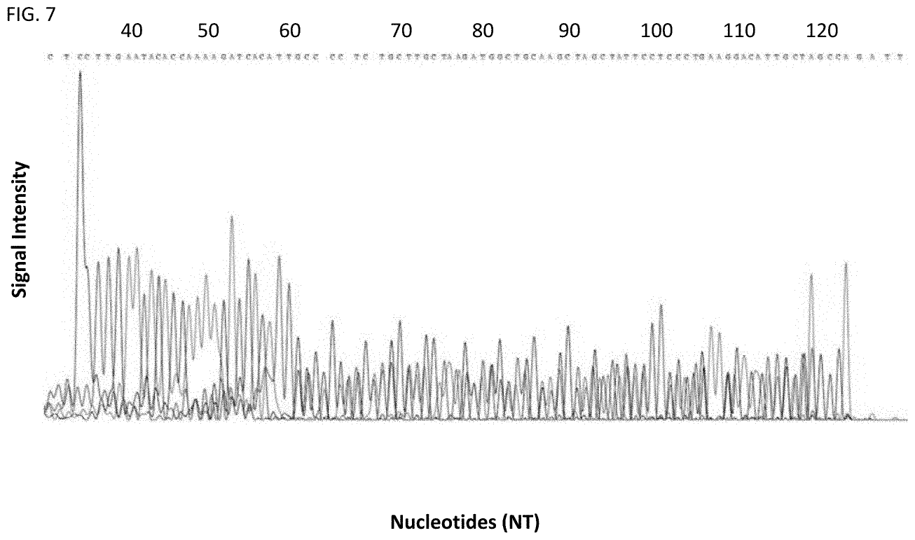

Sequencing operations preferably can be performed to determine one or more sequencing outputs (e.g., quantitative sequencing outputs upon which abundance metrics can be determined; etc.). Sequencing outputs can include any one or more of: chromatogram-related outputs, sequence reads, high throughput sequencing outputs, text data, alignments, and/or any other suitable outputs from any suitable sequencing technologies. Chromatogram-related outputs can include any one or more of chromatograms (e.g., including peaks for sequenced bases of target-associated molecule sequences, target sequences, reference-associated molecule sequences, reference sequences, of any suitable associated regions, of any suitable molecules described herein; etc.), alignment positions (e.g., corresponding to peaks and/or bases sequenced by Sanger sequencing; each alignment position corresponding to one or more peaks and/or one or more bases; corresponding to bases of a plurality of aligned sequences, such as bases of a target-associated sequence and a target sequence; as shown in FIGS. 6A and 7A; etc.), any suitable sequencing-related positions, peak intensities, peak areas, peak similarities, peak differences, peak metrics relative peaks for the same or different base type; average intensity; median intensity; heights; widths; overlap and/or other comparisons between peaks of target-associated base and a target base at the same position or at a different position, text data (e.g., text results from the Sanger sequencing, etc.), and/or any other suitable outputs (e.g., related to Sanger sequencing, etc.).

Sequencing outputs (e.g., in relation to peaks for Sanger sequencing) for one or more particular bases can include a dependency on the number and/or type of bases preceding (and/or are otherwise related to) the particular base, where addition of sequence regions (e.g., sequence repeats) in generating the spike-in mixture can account for such dependencies. Additionally or alternatively, determining a variation region sequence for a target-associated sequence and/or a reference-associated molecule sequence can be based on the dependencies (e.g., on the number and/or type of preceding bases in the sequence, etc.) and/or other suitable sequencing parameters (e.g., characteristics of the sequencing technologies, such as characteristics of Sanger sequencing, etc.), such as where predetermined insertion and/or deletions for variation regions can enable calibration (e.g., auto-calibration) for being able to accurately compare peak intensities and/or other suitable chromatogram-related outputs in facilitating abundance metric determination. For example, the target variation region includes at least one of one or more insertions and one or more deletions, where the at least one chromatogram-related output includes alignment positions corresponding to the peaks (e.g., associated with bases of the first target-associated region, the target sequence region of the biological target, the target variation region, and the sequence region of the biological target, etc.), where, for each of the different pairs (e.g., a base of the target-associated sequence and a base of the target sequence, etc.) the base of the first target-associated sequence corresponds to a first alignment position that is different from a second alignment position corresponding to the base of the first target sequence (e.g., as shown in FIGS. 6A and 7A), and where the alignment positions of the at least one chromatogram-related output include the first and the second alignment positions.

In an example, the variation region can include predetermined shuffled bases (e.g., base substitutions, etc.) that can enable calibration and/or suitable processing operations (e.g., deconvolution, correction factor determination and application, etc.) for improved accuracy in abundance metric determination. However, determination of any suitable region and/or sequence can be based on any suitable sequencing parameters in any suitable manner. Additionally or alternatively, sequencing outputs for a particular base and/or other sequence region, and/or determination of any suitable region and/or sequence can be independent of other sequence regions and/or suitable sequencing parameters.

In variations, performing one or more sequencing operations can be for one or more products (e.g., components of spike-in mixtures; target-associated molecules, target molecules and/or other suitable molecules; etc.) with added sequence regions (e.g., sequence repeats; etc.), such as one or more products generated based on hairpin sequences (e.g., based on amplification with PCR primers including one or more hairpin sequences; etc.). Performing sequencing operations on products with sequence repeats can function to sequence one or more sequences and/or sequence regions a plurality of times, for generating additional sequencing outputs, which can reduce noise, be used to determine additional abundance metrics (e.g., for determining an overall abundance metric of improved accuracy; etc.), and/or for any suitable purposes (e.g., facilitating characterizations and/or treatments; etc.).

However, performing sequencing operations can be performed in any suitable manner.

In some aspects of the present methods, the method can include determining one or more abundance metrics (e.g., for one or more samples; based on outputs of the one or more sequencing operations for the one or more spike-in mixtures, etc.), which can function to accurately determine abundance metrics, such as abundance metrics that can be meaningfully analyzed and compared (e.g., comparing individual abundances for a target molecule and a target-associated molecule to generate an abundance ratio, comparing abundance ratios for targets versus references; etc.), such as abundance metrics that can be used in facilitating characterizations and/or treatments. Abundance metrics can include any one or more of: abundance ratios (e.g., a ratio of first peak intensity metric for a first peak to a second peak intensity metric for a second peak, such as where the first and second peaks correspond to same or different alignment positions; ratios of any suitable sequencing output; a count ratio of an endogenous target molecule count to a target-associated molecule count; a sequencing output ratio of endogenous to spike-in, such as peak intensity metric ratio for a target sequence base and a corresponding target-associated sequence base; ratios with any suitable numerator and denominator; individual abundance ratios, such as usable in determining an overall abundance ratio and/or abundance metric; etc.), but can additionally or alternatively include individual abundances (e.g., individual peak intensities; counts; etc.), relative abundances, absolute abundances, and/or other suitable abundance metric. In a specific example, a ratio of endogenous molecules to spike-in molecules (e.g., ratio between endogenous DNA and spike-in DNA, etc.) can be calculated based on a sequencing output ratio (e.g., ratio of peak intensities for an endogenous-associated peak to a spike-in-associated peak) and a known abundance of spike-in molecules (e.g., used for generating the spike-in mixture).

In some embodiments, determining abundance metrics is based on one or more sequencing outputs, but can additionally or alternatively be based on any suitable data (e.g., supplementary data including known abundances, biometric data, medical history data, demographic data, genetic history, survey data, dietary data, behavioral data, environmental data, sample type, and/or other suitable contextual data). For example, determining abundance ratios (e.g., target-associated abundance ratios; etc.) (and/or any suitable abundance metrics) can be based on one or more chromatogram-related outputs including one or more peak intensities, peak areas, peak metrics for bases sharing a base type, peak metrics for bases with different base types and/or any other suitable chromatogram-related outputs and/or sequencing outputs. Determining abundance metrics can include computational processing (e.g., with a remote computing system such as a cloud computing system, with a local computing system, etc.), such as computationally processing one or more sequencing outputs (e.g., chromatograms, peak data, etc.) and/or other suitable data, but can additionally or alternatively include any suitable processing (e.g., manual processing; etc.). Processing (e.g., for determining abundance metrics; etc.) and/or suitable portions of embodiments of the method 100 (e.g., facilitating characterizations and/or treatments, etc.) can include any one or more of can include any one or more of: performing statistical estimation on data (e.g. ordinary least squares regression, non-negative least squares regression, principal components analysis, ridge regression, etc.), deconvolving (e.g., of overlapping peaks from a chromatogram, of peaks with inadequate resolution, of any suitable peaks; Fourier deconvolution; Gaussian function-based deconvolution; Lucy-Richardson deconvolution etc.), extracting features (e.g., for any suitable number of peaks of a chromatogram, etc.), performing pattern recognition on data, fusing data from multiple sources, combination of values (e.g., averaging values, etc.), compression, conversion (e.g., digital-to-analog conversion, analog-to-digital conversion), wave modulation, normalization, updating, ranking, validating, filtering (e.g., for baseline correction, data cropping, etc.), noise reduction, smoothing, filling (e.g., gap filling), aligning, model fitting, windowing, clipping, transformations, mathematical operations (e.g., derivatives, moving averages, summing, subtracting, multiplying, dividing, etc.), multiplexing, demultiplexing, interpolating, extrapolating, clustering, other signal processing operations, other image processing operations, visualizing, and/or any other suitable processing operations.

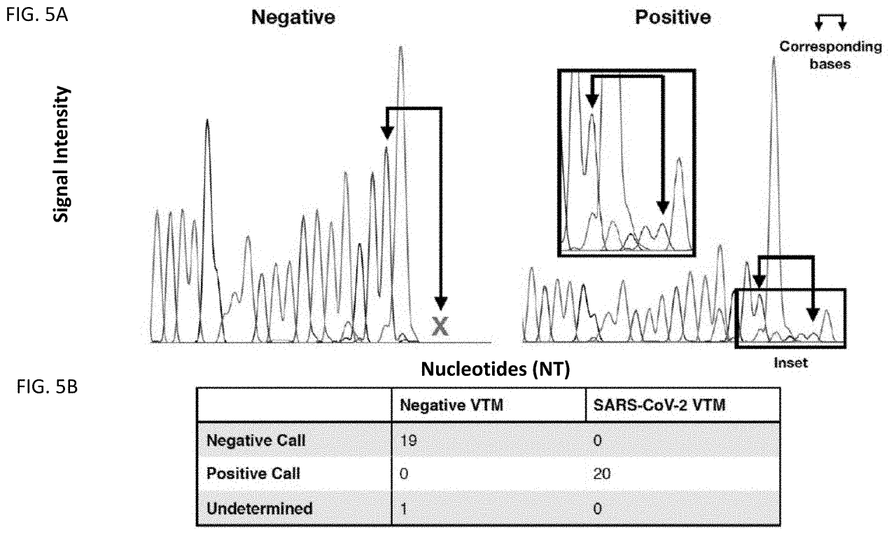

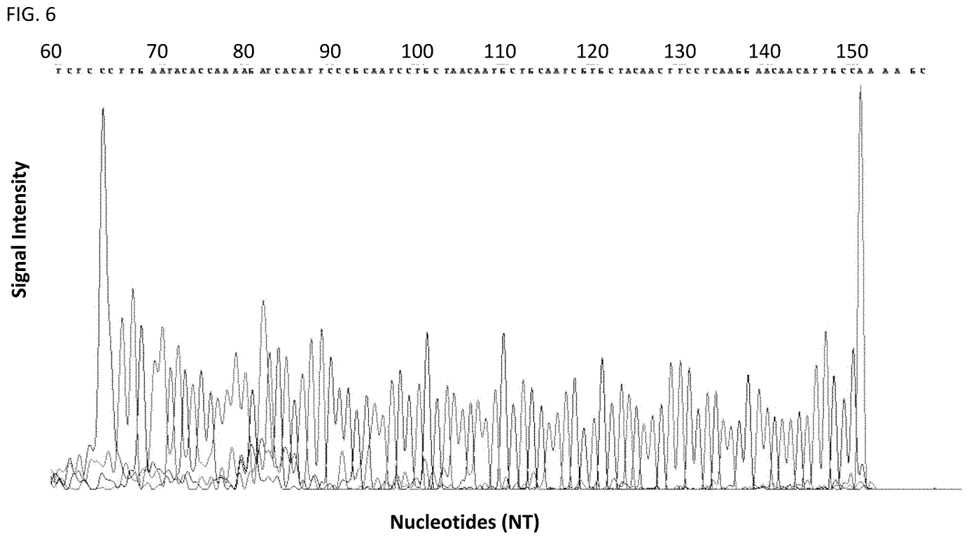

In variations, determining abundance metrics can be based on and/or otherwise associated with one or more sequencing outputs associated with synthetic target-associated sequences (and/or reference-associated sequences) including variation regions with one or more insertions (e.g., nucleotide insertions; etc.) and/or one or more deletions (e.g., nucleotide deletions; etc.) and/or any suitable modifications. For example, determining a set of target-associated abundance ratios can be based on a set of peaks (e.g., peak intensity data for peaks corresponding to sequenced bases of a target sequence and a target-associated sequence; etc.) and at least one of the substitution, the insertion, and the deletion (e.g., characteristics of the one or more substitutions, insertions, and/or deletions; such as size of the modification, in relation to number of nucleotides; types of modification such as in relation to base type changes; positions of where the modifications are applied; etc.). As shown in FIG. 5, variation regions including one or more insertions and/or deletions can result in shifted alignment positions (e.g., for bases of the target-associated sequence, relative bases a target sequence; such as where the sequence similarity between bases of a target-associated region and a target sequence region can be shifted in relation to position due to the one or more insertions and/or deletions; etc.). In an example, the target variation region (e.g., of a target-associated sequence; etc.) can include at least one of an insertion and a deletion, where the one or more chromatogram-related outputs can include alignment positions corresponding to a set of peaks (e.g., peak data for and/or associated with the target-associated region of the target-associated molecules, the target sequence region of the biological target, the target variation region of the target-associated molecules, and the sequence region of the biological target, etc.), and where determining the set of target-associated abundance ratios (and/or suitable abundance metrics) includes, for each of the different pairs (e.g., of a base of the target-associated sequence and a base of the target sequence; etc.): determining a peak intensity metric (e.g., a maximum intensity for the peak; an overall intensity for the peak; etc.), at a first alignment position of the alignment positions, for the base of the target-associated sequence of the pair, based on the at least one chromatogram-related output (e.g., based on peak intensity data for the sequenced bases; based on a chromatogram; etc.); determining a peak intensity metric, at a second alignment position of the alignment positions, for the base of the target sequence of the pair, based on the at least one chromatogram-related output (e.g., based on peak intensity data for the sequenced bases; based on a chromatogram; etc.), where the first alignment position is different from the second alignment position, and where the alignment positions include the first and the second alignment positions; and/or determining a target-associated abundance ratio (and/or suitable abundance metric; etc.) of the set of target-associated abundance ratios (and/or set of suitable abundance metrics; etc.), based on the peak intensity metric for the base of the target-associated sequence and the peak intensity metric for the base of the first target sequence. In an example, the first alignment position can correspond to a first peak and a second peak (e.g., overlapping peaks corresponding to the same alignment position; corresponding to same or different base types; etc.) of the first set of peaks, where the first peak corresponds to an overlapping base of the target-associated sequence, where the first peak corresponds to a first target-associated abundance ratio (e.g., for a pair of the overlapping base of the target-associated sequence and a corresponding base, shifted in alignment position, of the target sequence, where the amount of shift in alignment position is based on the characteristics of the one or more insertions and/or deletions, such as the sizes of the one or more insertions and/or deletions; etc.) of the set of target-associated abundance ratios, where the second peak corresponds to an overlapping base of the target sequence, and/or where the second peak corresponds to a second target-associated abundance ratio (e.g., distinct from the first target-associated abundance ratio; for a pair of the overlapping base of the target sequence and a corresponding base, shifted in alignment portion, of the target-associated sequence; etc.) of the set of target-associated abundance ratios.

However, determining abundance metrics based on and/or otherwise associated with sequencing outputs associated with variation regions including one or more insertions, deletions, and/or suitable modifications, can be performed in any suitable manner.

In certain embodiments, the method includes extracting abundance metrics. Extracting abundance metrics can include deconvolving overlapping peaks (e.g., chromatogram peaks, etc.) including a first peak corresponding to a target-associated sequence base (e.g., an “A” base) at a variation region position, and a second peak corresponding to a target sequence base (e.g., a “T” base) at the position; and calculating sequencing outputs and/or abundance metrics for the deconvolved peaks (e.g., a ratio of peak intensities for the “T” base to the “A” base). In a variation, deconvolution can be for overlapping peaks between a base of a first gene associated with e.g., Influenza A and a base of a second gene associated with Influenza B.

Relative and absolute abundance metrics is also described in U.S. Patent Application publication No.: 20190095577, which is hereby incorporated by reference in its entirety.

5.1.5.2 Fragment Analysis

In certain aspects of the present disclosure, the methods of the present disclosure include fragment analysis methods during the capillary electrophoresis step.

In some embodiments, fragment analysis is as an alternative approach for detection of infectious diseases. In certain embodiments, fragment analysis is similar to Sanger in that it is run via capillary electrophoresis (CE) using the same DNA Analyzer instrument. For example, the use of CE results in a measurable size separation of signals. Rather than labeling each nucleotide base as in Sanger sequencing, fragment analysis, in some embodiments, uses fluorescent end-point labeling wherein fluorescent dyes are attached to labeling primers and incorporated into samples through a PCR reaction.

In some embodiments, fragment analysis allows for target sample molecules to be separated by both size and/or color space. For example, a single injection can generate data for many independent loci. In certain embodiments, fragment analysis requires no more than two PCR reactions (amplification and labeling) and does not involve any bead purification as labeled product is directly diluted and denatured in a fixative (e.g., formamide) for injection during capillary electrophoresis.

In certain embodiments, fragment analysis allows for a multiplexed reaction to test for more than one infectious disease, and the co-amplified mixtures can be run on a single capillary.

In certain embodiments, fragment analysis allows for a singleplexed reaction to test for a single infectious disease.

In certain embodiments, the fragment analysis after capillary electrophoresis allows for testing the presence or absence of about 1-50 infectious diseases, such as 1-10, 1-5, 30-50, 10-20, 1-10, 5-10, 1-3, 1-4, 1-5, 1-25, or 25-50 infectious diseases. In certain embodiments, performing capillary electrophoresis can be carried out on a single capillary. In certain embodiments, performing capillary electrophoresis can be carried out on a 1 or more, 2 or more, 3 or more, 4 or more, 5 or more, 6 or more, 7 or more, 8 or more, 9 or more, or 10 or more capillaries.

In certain embodiments where the method includes detecting the presence or absence of more than one infectious disease, the synthetic target-associated molecules will also include two or more, three or more, four or more, five or more, six or more, seven or more, eight or more, nine or more, or ten or more target-variation regions, where each target-variation region is distinguishable from a second region of a different infectious disease's RNA or DNA. In certain embodiments, the synthetic target-associated molecules will include 1-50, 30-50, 10-20, 1-10, 5-10, 1-3, 1-4, 1-5, 1-25, or 25-50 target-variation regions, where each target-variation region is distinguishable from a second region of a different infectious disease's RNA or DNA. Thus, in one aspect, the methods of the present disclosure can test for about 1-50 pathogens or infectious diseases. For example, a first target-variation region can have a nucleotide sequence with an insertion or deletion as compared to a corresponding nucleotide sequence in the second region of the Influenza A virus's RNA or DNA RNA, a second target-variation region can have a nucleotide sequence with an insertion or deletion as compared to a corresponding nucleotide sequence in the second region of the Influenza B virus's RNA or DNA RNA, a third target-variation region can have a nucleotide sequence with an insertion or deletion as compared to a corresponding nucleotide sequence in the second region of the SARS-CoV-2 virus's RNA or DNA RNA.

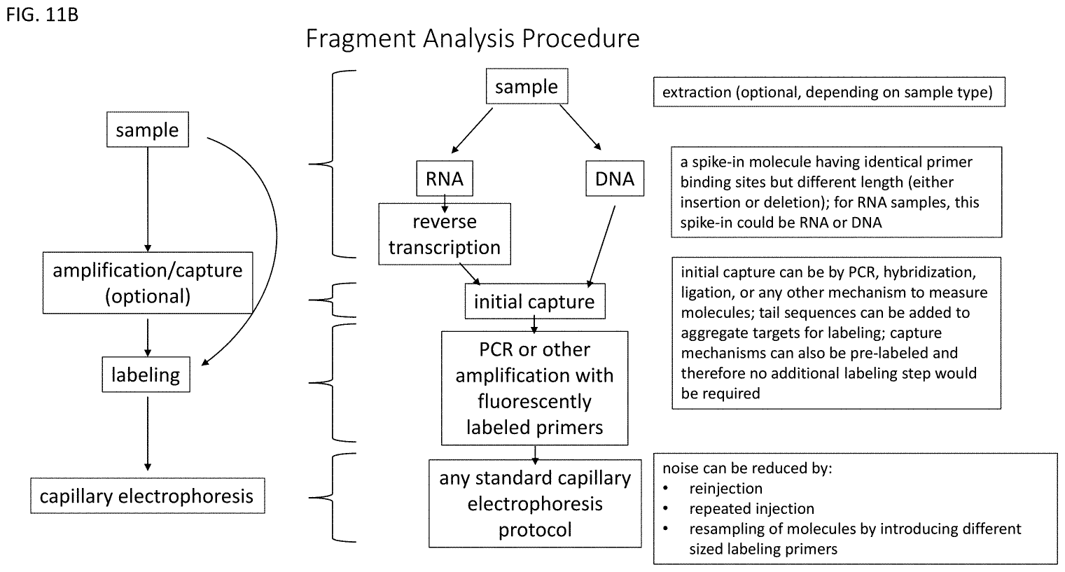

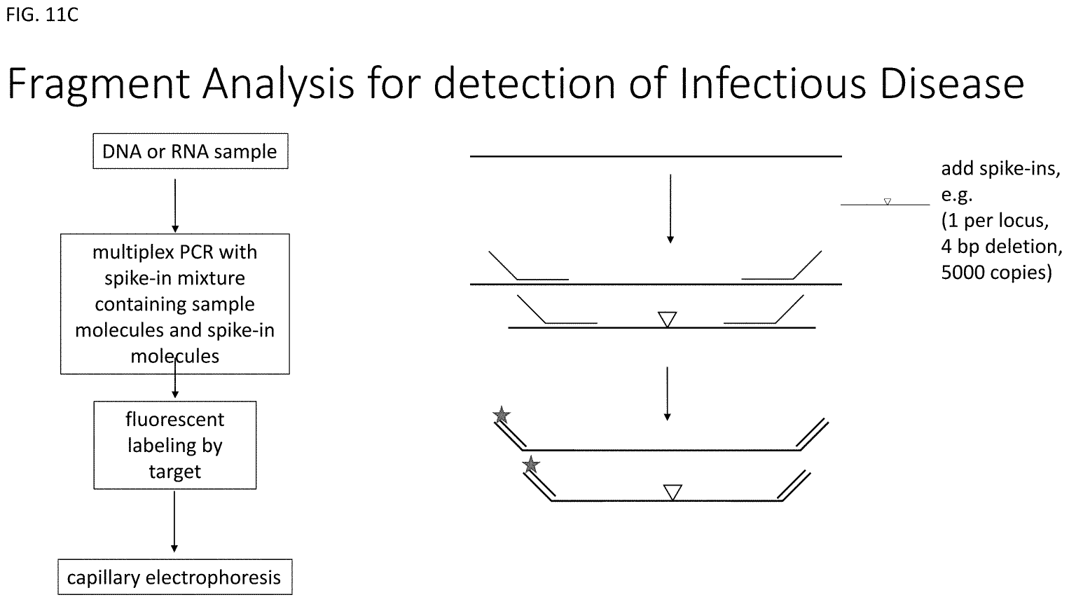

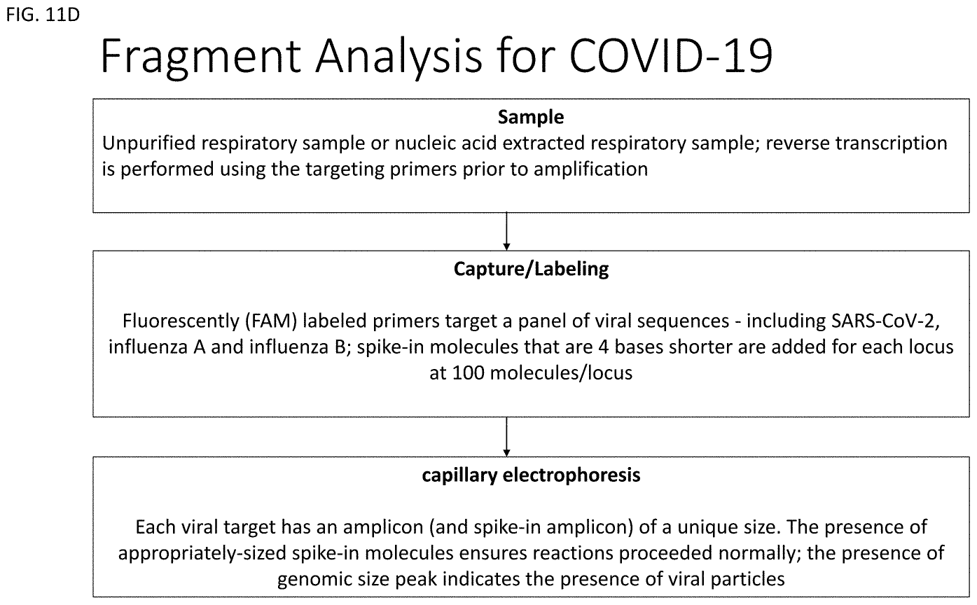

In the method shown in FIG. 11B, a genomic sample is optionally extracted, depending on the sample type. The sample may be a DNA sample or an RNA sample. If the sample is an RNA sample, a reverse transcriptase (RT) may be used to generate DNA (DNA) from the RNA. Genomic samples are extracted through any appropriate sample extraction mechanism. A spike-in molecule synthetic target-associated molecule is associated/matches with a biological target (infectious disease DNA or RNA) and is mixed with the extracted sample. The mixture of the genomic sample and the spike-in molecule are captured (using a plurality of forward and reverse primers complementary to the target-region of the synthetic target associated sequence and complementary to a region of the infectious disease's RNA or DNA) and are amplified. Amplification may be performed via any suitable mechanism, such as polymerase chain reaction (PCR), singleplexed PCR, multiplexed PCR, multiplexed tailed PCR, reverse-transcription PCR (PT-PCR), hybridization, ligation, or any other mechanism to measure molecules (e.g., “initial capture” and amplification of FIG. 11B). Initial capture can be performed by any suitable mechanism, such as PCR, reverse-transcription PCR (PT-PCR), hybridization capture, ligation, or any other mechanism to measure molecules.

In some embodiments, during the initial capture, tail primer sequences may be added, to the co-amplified spike-in mixture, e.g., to re-use and/or resample amplicons, measure multiple amplicons simultaneously, aggregate amplicons of the same type of length (e.g., synthetic target associated molecules, first set of target sample molecules, second set of target sample molecules, etc.), facilitate labeling of fluorescent primers, or the like, which may help reduce noise. For example, fluorescently labeled primers may be used to tag amplicons with different fluorophores such that the same amplicon may be measured across different color channels. Data can then be aggregated for the same amplicon across the different channels to reduce noise. Similarly, primers may be used to add tail-end sequences of different lengths to an amplicon such that the same amplicon may be measured multiple times across one or more color channels. For example, tails with a length of 1-10 nucleotides, 10-20 nucleotides, 20-30 nucleotides, 30-50 nucleotides, and the like, may be added to the amplicons of the target sequence and the spike-in synthetic target-associated sequence.

Moreover, primers of various lengths may be used to measure multiple separate amplicons simultaneously. In one embodiment, multiple separate amplicons may be measured simultaneously by labeling separate amplicons with different fluorophores. For example, for a first infectious disease, the target sequences and corresponding spike-in sequences may be labeled with a fluorophore that emits blue light. For a second infectious disease, the target sequences and corresponding spike-in sequences may be labeled with a fluorophore that emits red light. Alternatively, or additionally, tails of various lengths may be added to the amplicons corresponding to each infectious disease's DNA or RNA, each of which has been tagged with a different fluorophore. Thus, the amplicons of various sizes may be aggregated across each size but within a color channel. This enables multiple separate amplicons to be measured simultaneously while resampling, which may reduce noise.

In one aspect of the present methods where fragment analysis is used as a mode of capillary electrophoresis, the method includes co-amplifying the spike in mixture with primers that are complementary to the target-matching region of the synthetic target-associated molecules and that hybridize to the first region of the infectious disease's RNA or DNA. In certain embodiments, the set of primers further include universal tailed-primers. In certain embodiments, the set of primers further comprise fluorescently labeled primers that include one or more fluorescently labeled tags. In certain embodiments, the fluorescently labeled tags are attached to the 5′ or 3′ end of the primers. In certain embodiments, the fluorescently labeled tags are attached to the 5′ end of the primers. In certain embodiments, the fluorescently labeled tags are attached to the 3′ end of the primers. In other embodiments, the set of primers comprise primers that are complementary to the target-matching region of the synthetic target-associated molecules and that hybridize to the first region of the infectious disease's RNA or DNA, wherein the primers further comprise a fluorescently labeled tag.

In some embodiments, after co-amplification of the spike in mixture, the spike-in mixture is co-amplified and generates amplicon products of the synthetic target associated molecules and amplicon products of the sample molecule.

In some embodiments, an amplicon product generated by amplifying a given infectious disease's RNA or DNA differs by a predetermined length from an amplicon product generated by amplifying the corresponding target matching and target variation regions of the synthetic target-associated molecules.

In some embodiments, sample amplicon products associated with a first infectious disease have a sample nucleotide length that is different by a predetermined amount than a sample nucleotide length of the sample amplicon products associated with a second infectious disease and the sample amplicon products associated with a third infectious disease.

In some embodiments, the synthetic target-associated amplicon products comprise a first set of target-associated amplicon products comprising the first target-matching region and the first target-variation region. In certain embodiments, the synthetic target-associated amplicon products comprise a second set of target-associated amplicon products comprising the second target-matching region and the second target-variation region, wherein the first set of target-associated amplicon products comprise a fluorescent label that is distinct from a fluorescent label of the second set of target-associated amplicon products.

In some embodiments, the sample amplicon products comprise a first set of sample amplicon products for detecting a first infectious disease and a second set of sample amplicon products for detecting a second infectious disease, where the first set of sample amplicon products comprise a fluorescent label that is distinct from a fluorescent label of the second set of sample amplicon products.

In some embodiments, the first set of sample amplicon products and the first set of target-associated amplicon products comprise the same type of fluorescent label. In some embodiments, the second set of sample amplicon products and the second set of target-associated amplicon products comprise the same type of fluorescent label. In some embodiments, the first set of sample amplicon products and the first set of target-associated amplicon products comprise the same type of fluorescent label. In some embodiments, the second set of sample amplicon products and the second set of target-associated amplicon products comprise the same type of fluorescent label.

In certain embodiments, the synthetic target-associated amplicon products comprising a nucleotide length that is shorter than the nucleotide length of the nucleotide sequence of the first infectious disease or the nucleotide sequence of the sample molecule. In some embodiments, the nucleotide length of the synthetic target-associated amplicon products is shorter by 1-50 nucleotides (e.g., shorter by 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 20 or more, 25 or more, 30 or more, 35 or more, 40 or more, 45 or more, or 50 or more nucleotides). In some embodiments, the co-amplified mixture comprises synthetic target-associated amplicon products comprising a nucleotide length that is longer than the nucleotide length of the nucleotide sequence of the first infectious disease or the nucleotide sequence of the sample molecule. In certain embodiments, the nucleotide length of the synthetic target-associated amplicon products is longer by 1-50 nucleotides (e.g., longer by 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 20 or more, 25 or more, 30 or more, 35 or more, 40 or more, 45 or more, or 50 or more nucleotides).

In some embodiments, co-amplifying occurs in a single amplification step. In certain embodiments, co-amplifying occurs in two or more amplification steps. In certain embodiments, an initial capture prior to co-amplification using a hybridization capture approach.

Following co-amplification, capillary electrophoresis is performed on the amplified and labeled spike-in mixture. Any suitable capillary electrophoresis protocol may be used.

In some embodiments, the methods of the present disclosure include performing capillary electrophoresis on the co-amplified spike-in mixture to determine a chromatogram-related output comprising one or more chromatogram intensities.

In some embodiments, the method includes determining the presence or absence of at least one infectious disease by comparing the chromatogram intensity associated with the amplicon product generated by amplifying the at least one infectious disease's RNA or DNA and a chromatogram intensity associated with an amplicon product having a length that differs by the predetermined length from the amplicon product generated by amplifying the at least one infectious disease's RNA or DNA.

In some embodiments, the method includes determining the presence or absence of first infectious disease by comparing the chromatogram intensities associated with the amplicon products having the target-associated nucleotide length and amplicon products having the sample nucleotide length.

In some embodiments, the plurality of chromatogram intensities include an intensity peak associated with: the target-matching region of the synthetic target-associated molecules; the target-variation region of the synthetic target-associated molecules; and a region of the sample molecules of the subject that corresponds to the target-variation region of the synthetic target-associated molecules. In some embodiments, the method further comprises determining the presence or absence of first infectious disease by comparing the chromatogram intensity peaks associated with the first target-variation region of the synthetic target-associated molecules and the region of the sample molecules of the subject.

In certain embodiments, intensity can include intensity or amplitude height, intensity or amplitude depth, intensity or amplitude area, intensity or amplitude area under the curve, intensity or amplitude peaks, or a combination thereof.

In some embodiments, the chromatogram intensities comprise one or more intensity peaks. In some embodiments, the chromatogram intensities comprise one or more fluorescence intensity peaks.

In some embodiments, the one or more intensity peaks of the synthetic target-associated amplicon products is associated with a nucleotide length of the synthetic target-associated amplicon products, and wherein the one or more intensity peaks of the sample amplicon products is associated with a nucleotide length of the sample amplicon products.

In some embodiments, each chromatogram intensity comprises one or more peak intensities associated with: the target-associated region of the target associated amplicon product; the target variation region of the target associated amplicon product; or the target region of the sample amplicon product of the subject. In some embodiments, the method further comprises comparing comprises calculating the ratio of peak intensities of the sample amplicon products to the peak intensities of the synthetic target-associated amplicon products.

In certain embodiments, comparing the chromatogram intensities further comprises calculating/computing the ratio between the intensity peak associated with the first target-variation region of the synthetic target-associated amplicon products and intensity peak of the region of the sample amplicon products of the subject.

In some embodiments, intensity peaks associated with a region of the target sample amplicon products include intensity peaks that match the target-variation region, e.g. if an infectious disease's RNA or DNA is present in the target sample amplicon products, but are offset (e.g., shifted left or right, being a predetermined nucleotide distance away) as compared the intensity peaks of the target-variation region because of the insertion or deletion of the target-variation region. Relative or absolute abundances of the target-associated molecules and the infectious disease's RNA or DNA can be determined from the relative size of the offset peaks. In some embodiments, peak intensity of the region of the sample molecules that corresponds to the target-variation region of the synthetic target-associated molecules includes a peak intensity position that is offset as compared to the peak intensity position of the target-variation region, wherein the peak intensity position is offset by one or more nucleotides associated with the insertion or deletion of the target-variation region.

In certain embodiments, the method further comprises: aggregating peak intensities across each synthetic target-associated amplicon products of the same nucleotide length; aggregating peak intensities across each sample amplicon products of the same nucleotide length. In some embodiments, the method further comprises computing a ratio between the aggregated sample amplicon product peak intensity and the aggregated synthetic target-associated amplicon product peak intensity.

Data, such as intensity (e.g., intensity or amplitude height, intensity or amplitude depth, intensity or amplitude area, intensity or amplitude area under the curve, intensity or amplitude peaks, and the like) is received from the capillary electrophoresis. Data may be aggregated in any suitable manner across size and/or color channels. The data of both the target sample molecule and the spike-in synthetic target-associated molecules may be used to determine absolute and/or relative abundances of the biological target in the genomic sample. Absolute abundances may be estimated by comparing the data of the target sample molecule peaks to spike-in synthetic associated-molecule peaks.

Relative abundances of alleles may be estimated if the alleles differ in length. The ratio of the spike-in synthetic target-molecule peaks and the target sample molecule peaks may be used to estimate dosage, discussed in detail below.

Data may then be aggregated based on the length of the amplicon products of the co-amplified spike in mixture during amplification to compute ratios between the respective target sample molecule peak intensity of the target sequence and the respective spike-in peak intensity of the spike-in synthetic target-associated sequence for each target-variation region. The presence or absence of the infectious disease is determined based on the computed ratios.

In some embodiments, presence or absence of the infectious disease is determined by computing the ratio of an infectious disease-specific ratio to each of the other infectious disease-specific ratios. For example, the ratio of the target sequence to the spike in sequence is computed for each infectious disease, such Influenza A, Influenza B, and SARS-CoV-2. Then, ratios between a particular disease-specific ratio and each of the other disease-specific ratios are computed. As a non-limiting example, in determining the presence or absence of an infectious disease, the Influenza A: Influenza B ratio, Influenza A: SARS-CoV-2 ratio, Influenza A: MERS-CoV ratio, and Influenza A: SARS-CoV ratio are computed. As another non-limiting example, in determining the presence or absence of an infectious disease, the SARS-CoV: SARS-CoV-2 ratio, and SARS-CoV: MERS-CoV ratio are computed. The presence or absence of the infectious disease may be predicted based on a comparison of these ratios.

### 6. EXAMPLES

### 6.1. Example 1: Pilot Study for a qSanger Assay Approach for COVID-19

Described herein is a molecular diagnostic for COVID-19 based on Sanger sequencing. This assay used the addition of a frame-shifted spike-in, a modified PCR master mix, and custom Sanger sequencing data analysis to detect and quantify SARS-CoV-2 RNA at a limit of detection comparable to existing qPCR-based assays, at 10-20 genome copy equivalents. The assay was able to detect SARS-CoV-2 RNA from viral particles suspended in transport media that was directly added to the PCR master mix, suggesting that RNA extraction can be skipped entirely without any degradation of test performance. Since Sanger sequencing instruments are widespread in clinical laboratories and commonly have built-in liquid handling automation to support up to 3840 samples per instrument per day, the widespread adoption of qSanger COVID-19 diagnostics can unlock more than 1,000,000 tests per day in the US.

The workflow of qSanger-based COVID-19 is distinguished from Sanger sequencing of reverse transcription (RT)-PCR amplicons (FIG. 2), by including the addition of a frame-shifted synthetic COVID-19 spike-in DNA in the reaction master mix. qSanger COVID-19 is designed to support one-step reverse-transcription (RT)-PCR directly from viral transport media (VTM) of specimens, without an RNA purification step (FIG. 1A). The amplification products are then purified, and Sanger sequenced by automated capillary electrophoresis. Synthetic DNA included in RT-PCR master mix prior to PCR amplification serves as an internal control that enables specimens to be readily identified as either positive or negative for COVID-19 (FIG. 2B-2C). Quantitative analysis of the Sanger sequence chromatogram gave qSanger an extremely high sensitivity and specificity for all positive results with a limit of detection of 10-20 genome copy equivalents (GCE), equivalent to gold-standard qPCR methods. Furthermore, the presence of a spike-in as an intra-sample control in the qSanger assay allows for easy interpretation of results and determination of sources of error (e.g. extraction or amplification or sequencing failure), and also allows population-level analyses such as mutational analysis and contact tracing. In addition, the ratio of the amplitudes of corresponding bases between the endogenous and spike-in sequences at offset positions reflects the ratio of the molecular abundances of the two sequences. Computationally combining the amplitude ratios of multiple corresponding bases can then be used to estimate the viral load over a 400-fold dynamic range with Poisson-limited coefficient of variation.

Results

qSanger COVID-19 Limit of Detection is comparable to RT-qPCR.

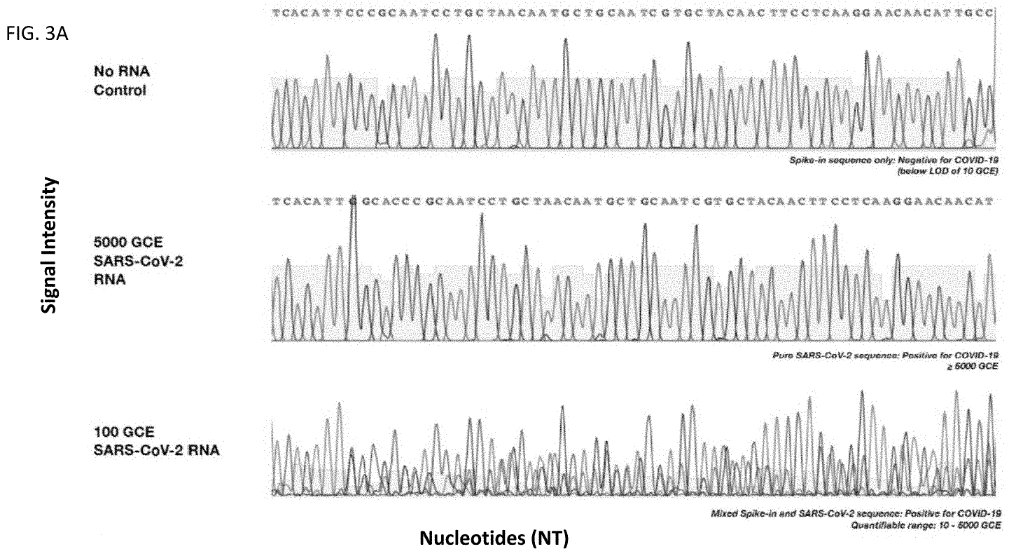

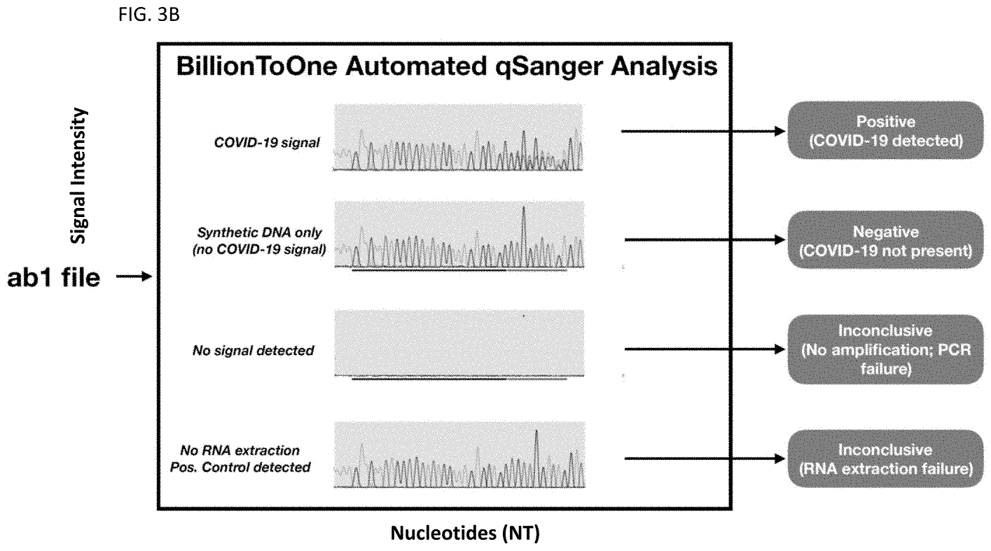

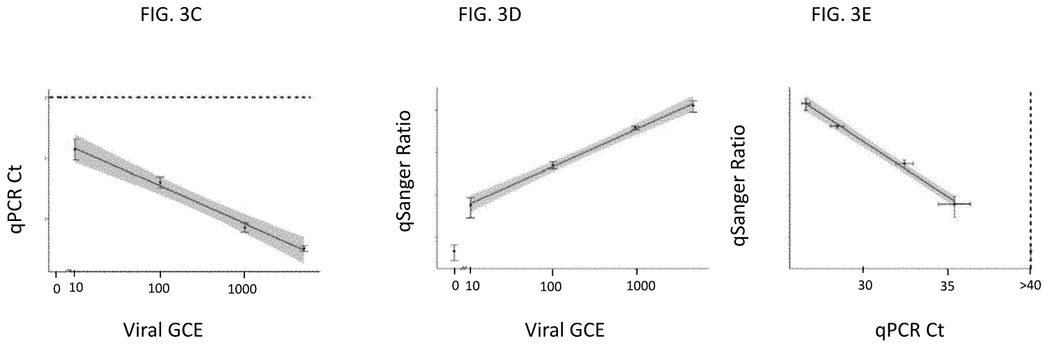

As an initial demonstration of qualitative detection of COVID-19 by qSanger PCR primers and synthetic DNA spike-in were designed to target SARS-CoV-2 N protein (FIG. 2B). A one-step RT-PCR mix (NEB) containing both was used to perform reverse-transcription of SARS-CoV-2 RNA and subsequent PCR amplification in one pot. In each RT-PCR, either nuclease-free water as a no-template control (NTC) or 100-5000 GCE of synthetic SARS-CoV-2 RNA (Twist Biosciences) was added. All reactions also contained ˜200 GCE of spike-in in the RT-PCR master mix. After RT-PCR and Sanger sequencing, a qualitatively clean chromatogram was observed for the spike-in sequence for the NTC condition in which no SARS-CoV-2 RNA was added (FIG. 3A). At the 100 GCE level, the Sanger chromatogram showed clear mixed bases corresponding to approximately equal abundance of spike-in DNA and SARS-CoV-2 RNA. At 5000 GCE SARS-CoV-2 RNA, the chromatogram exhibited a relatively pure trace for the SARS-CoV-2 target sequence, suggesting that the SARS-CoV-2 signal overwhelmed the spike-in signal when it was present at 50-fold greater abundance.

To determine the limit of detection of qSanger COVID-19, assays were performed on dilutions of SARS-COV-2 RNA corresponding to 0 GCE, 10 GCE, 100 GCE, 1000 GCE, or 5000 GCE. Four replicates at each dilution were assayed by both qSanger and qPCR. As expected, all four replicates of 0 GCE were negative for COVID-19 by qPCR, and addition of 10 or more SARS-CoV-2 GCE exhibited a clear logarithmic decrease in qPCR cycle threshold (Ct) (FIG. 3C). Similarly, no SARS-CoV-2 sequence was apparent on Sanger chromatograms for the NTC condition. No spike-in sequence was qualitatively discernable at the 5000 GCE dilution. Mixed bases were obviously present for both the 10 GCE and 100 GCE conditions suggesting that the limit of detection is about 10 GCE SARS-CoV-2.

A custom bioinformatic analyses was developed to extract the relative abundance of SARS-CoV-2 and spike-in amplified products from Sanger chromatograms and automate analysis of qSanger chromatograms (see Methods). Briefly, peak amplitudes were assigned to either the spike-in or SARS-CoV-2 sequence at each base position, and a linear regression analysis was performed to determine the qSanger ratio between SARS-CoV-2 and spike-in trace intensities. qSanger ratios near 0 were recovered in the samples with 0 GCE, indicating the complete absence of SARS-CoV-2 RNA. (FIG. 2C). Since all of the SARS-CoV-2 RNA at 10 GCE or qSanger ratios of 3% or greater, this provided further evidence that the limit of detection of qSanger COVID-19 is ˜10 GCE or fewer. Quantitative analysis of chromatogram peak heights was able to recover a qSanger ratio for even the 5000 GCE condition, and the qSanger ratios had excellent linearity over 10-5000 GCE (FIG. 3D). Furthermore, qSanger ratios were in good agreement qPCR Ct values (FIG. 3E).

qSanger Detects SARS-CoV-2 RNA without RNA Purification

Since a major limitation for increasing testing capacity has been supply chain and lab workflow bottlenecks related to RNA extraction, it was next attempted to detect SARS-CoV-2 directly from the specimen matrix (viral transport medium). There has been a previous report that RT-qPCR can be successfully performed when up to 3-7 ul (total reaction volume of 20 ul) of VTM without extraction is used as the template for RT-PCR [9]. It was hypothesized that qSanger could be a more reliable method for detection of COVID-19 without RNA extraction because of i) increased robustness against PCR inhibitors in the specimen matrix since quantification of SARS-CoV-2 is performed via comparison with the spike-in internally control; ii) an improved limit of detection by adding more VTM to a correspondingly larger reaction size; and, iii) avoidance of false-positives by examining sequencing data for the correct spike-in or SARS-CoV-2 sequence. To test this hypothesis, reference materials were obtained in which either SARS-CoV-2 (positive control) or human RNA (negative control) is packaged inside of viral particles and suspended in VTM (Seracare). Since polymerase mixes can have varying resiliency to PCR inhibitors, both Luna RT-qPCR and OneTaq RT-PCR kits were evaluated.

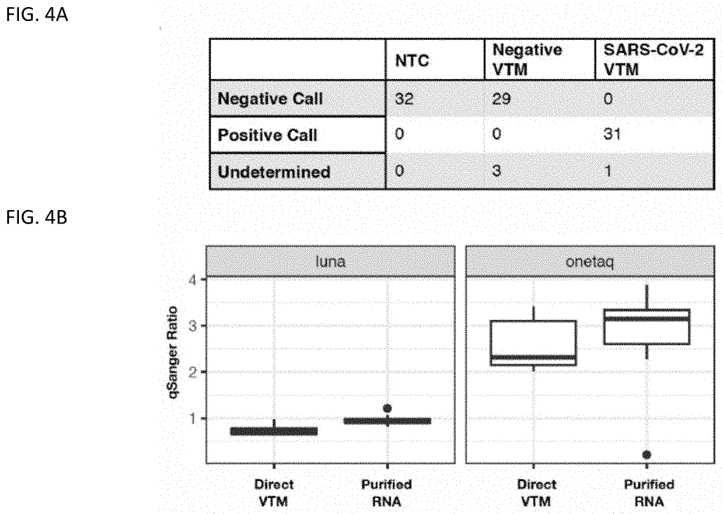

For both Seracare negative and positive control specimens, eight replicates each were performed on the cross-product of conditions for Luna vs OneTaq polymerases and direct VTM vs purified RNA, for 64 reactions total. 25 ul of the Seracare specimen (corresponding to 125 GCE) was added to a 100 ul total reaction volume. Additionally, 16 replicates of no-template controls were performed for each polymerase wherein nuclease free water was added to the reaction. All 32 NTC samples across all conditions were negative by qSanger assay and analysis (FIG. 4A). Nearly all Seracare negative control samples were determined to be negative by qSanger; indeterminate results were obtained from two purified RNA Luna specimens and one purified OneTaq specimen. All Seracare positive controls were identified by qSanger except for a OneTaq direct VTM specimen (FIG. 4A). Samples were classified as undetermined due to low chromatogram signal intensity (Signal to noise ratio <500) or lack of sequence alignment. Possible reasons for undetermined chromatograms could be failure in RNA extraction, PCR amplification, or cycle sequencing. Since the majority of undetermined specimens used purified RNA, the possibility that the majority of assay failures were due to the RNA extraction process itself, perhaps by carryover of high salt buffers, should be considered.

To further evaluate the feasibility of a direct VTM, extraction-free method for Sars-CoV-2 detection, the ability of qSanger was also examined to quantify the amount of viral particles in the Seracare positive control specimens (FIG. 4B). Since each reaction contained 125 GCE of Sars-CoV-2 and 200 GCE of spike-in, it was expected to measure a qSanger ratio of 0.625.

OneTaq results yielded a qSanger ratio consistently around 2-3.5. This discrepancy could be due to a slightly more efficient amplification of the SARS-CoV-2 sequence compared to spike-in sequence. Remarkably, Luna polymerase mix yielded a qSanger ratio of 0.74±0.04 s.e.m. for VTM and a qSanger ratio of 0.97±0.04 s.e.m. for purified RNA, which is very close to the expected ratio. The ˜20% difference is on par with typical imprecisions for pipetting and DNA quantification. The coefficient of variation (CV) for the VTM and RNA purified Luna assays were 12% and 16%, respectively. Notably, this is in good agreement with the imprecision associated with measuring ˜125 molecules at the Poisson limit. Since Luna exhibited better accuracy and precision compared to OneTaq, and the Luna direct VTM method resulted in correct calls for all NTC, Seracare positive, and negative specimens without any failed reactions, subsequent experiments were performed with Luna polymerase mix.

Finally, it was demonstrated that omitting RNA extraction does not adversely affect qSanger sensitivity. 20 GCE (corresponding to 2× the LOD in FIG. 2) of viral particles was added in VTM containing either negative control or SARS-CoV-2 RNA (FIG. 5). Sanger chromatograms clearly showed the absence and presence of SARS-CoV-2 signal in the negative and positive controls, respectively (FIG. 5A). The qSanger assay correctly identified the negative and positive samples in 39 out of the 40 samples tested, with one negative control specimen returning an undetermined result due to sequencing failure (FIG. 5B). Overall, the excellent performance shown by our qSanger results on unextracted VTM vs. purified RNA with respect to absolute quantification accuracy, Poisson-limited coefficient of variation, and limit of detection that is comparable to gold-standard RT-qPCR, suggests that qSanger can be performed on unprocessed specimen matrix without any loss in performance. In fact, it might be possible that RNA-extraction free methods are more reliable because it eliminates the carryover risk of high-salt, PCR-inhibiting buffers used in RNA extraction procedures.

Discussion

The disclosed qSanger assay can detect COVID-19 without RNA extraction. The qSanger assay performs as well as qPCR in estimates of viral RNA abundance and consistently detects as low as 10-20 viral genome copy equivalents, even when VTM is added directly into the reaction mix without RNA extraction. Because qSanger is an end-point PCR reaction with an internal spike-in control, it is more robust to inhibitors that can exist in VTM, and failures in amplification result in undetermined results that require a repeat reaction, as opposed to false negatives that would be obtained by qPCR. It also has higher specificity than qPCR, as the examination of the sequencing information can be used to distinguish similar viruses and rule out false-positives due to non-specific amplifications.

Since qSanger can have an extremely high specificity enabled by the sequencing information, it can be used for routine testing of asymptomatic individuals with high positive predictive value (PPV). This can be a new paradigm of routine and repeated testing of individuals who are at high risk for contracting disease, e.g. hospital staff or those who are older or with comorbidities. Early detection can improve individual healthcare outcomes and also enable relaxation of population scale non-pharmaceutical interventions like social distancing measures.

In addition not requiring RNA extraction kits, the qSanger-based COVID-19 assay has a number of additional advantages compared to existing qPCR-based tests for COVID-19. QSanger thermal cycling occurs in higher-throughput end-point PCR instruments, rather than specialized qPCR instruments, and the sequencing can be run in automated Sanger sequencers with plate feeders such as Applied Biosystems 3730xl DNA Analyzers which have the capacity to sequence 3840 samples per day. The large existing install-base of end-point PCR and high-throughput Sanger instruments throughout the US and the world [14] supports rapid scale-up of qSanger-COVID-19 assays without requiring any new device or instrument manufacturing. Given that Sanger sequencing is still the most widely used method of clinical sequencing worldwide, the widespread adoption of qSanger-COVID-19 assay described here can create >1M COVID-19 testing capacity per day.

More broadly, qSanger can enable an even higher volume of population-scale testing if clinical laboratories and Sanger sequencing centers are allowed to collaborate for COVID-19 testing. While Sanger sequencers exist in all molecular diagnostic laboratories, they are most commonly utilized in high volume in genome centers, academic sequencing core facilities, and commercial Sanger sequencing service laboratories. In this model, clinical laboratories could buy 96-well master-mix reaction plates that simply require the addition of each patient sample into a reaction well in a BioSafety Cabinet and PCR thermocycling, whereas the sequencing service laboratory would sequence the samples for next-day results. This would enable a rapidly deployed and distributed population-scale testing.

qSanger also has a number of other advantages that may prove to be invaluable as more information is learned about SARS-CoV-2 and other infectious diseases. Since SARS-CoV-2 mutates quickly, the availability of sequence information can be used to identify growing clusters of mutations and aid with contact tracing via phylogenetic analysis of the mutation data. Longer sequences can be designed to capture a wide range of mutations in the qSanger reaction, as the virus mutates and creates sub-strains with different clinical implications.

Moreover, as opposed to relative measurements obtained by qPCR, absolute measurements of viral load are obtained in qSanger due to the known molecular count of the spike-in. Quantification of viral abundance in a sample may prove to be useful for determining who is infectious, as well as for more accurate environmental monitoring. The quantitative dynamic range of qSanger can be broadened from 0-5000 GCE to 10-2,500,000 GCE by employing two qSanger reactions with different molecular levels of spike-ins.

Methods

Primer and Spike-in

Spike-in sequences were designed using the viral genomic region corresponding to the CDC designed N3 qPCR assay. Spike-in molecules have sequences identical to SARS-CoV-2 sequence (LC528232) including base positions 28216 to 29280 but lacking bases 28715-28718.

Primers flanking the deletion were used for amplification. Sequencing was performed using a primer containing the forward amplification binding region. See forward and reverse spike in sanger sequencing primer in Table 1.

TABLE 1
Primers (Top to Bottom: SEQ ID NO: 16-18)

| Name | Purpose | Sequence |
|---|---|---|
| short_N_spk_2_F | PCR F | 5′-AAGACGGCATCATATGGG |
| | | TTGC-3′ |
| short_N_spk_1_R | PCR R | 5′-GGCAATGTTGTTCCTTGA |
| | | GGAAG-3′ |
| short_N_spk_2_ | Spanger | 5′-CCGTAACGTGGCACTGGA |
| F_wBarcode_ | Sequencing | CCACTACTAGGCGTTACAGCT |
| CCGTAA | | TCAACACCTGGAAGACGGCAT |
| | | CATATGGGTTGC-3′ |

Samples

Synthetic SARS-CoV-2 genomic RNA from Twist Biosciences was used for RNA detection linearity experiments. AccuPlex SARS-CoV-2 Reference Material Kit manufactured by Seracare (cat. #0505-0126) was used as a proxy for clinical samples.

Viral Purification

Viral purification was performed using PureLink Viral RNA/DNA Mini Kit from ThermoFisher Scientific using 500 μL (at 5000 GCE/mL) of Accuplex Positive or Negative samples. GCE input estimates from purified RNA were estimated by corresponding fraction of eluent assuming 100% recovery.

qPCR

qPCR were performed using the N1 primers and TaqMan probes provided in the 2019-nCoV RUO Kit manufactured by IDT (cat. #10006605). Amplification was performed as described in the CDC EUA protocol. Briefly, 2 μL of synthetic RNA template diluted in RNAse-free TE+0.05% Tween-20 to 5, 50, 500, or 2500 GCE/μL were added to each reaction. RNA samples were combined with water, TaqPath 1-Step RT-qPCR Master Mix, primers (1.5 μL to a final concentration of 500 nM), and probes to a total final volume of 20 μL. The reaction mixture was amplified and probe fluorescence was detected using a Mastercycler ep realplex Real-time PCR System. The first cycle above threshold was estimated (Ct) was performed with default settings using realplex software.

qSanger Amplification

Reverse transcription and amplification for FIG. 3 was performed using OneTaq One-Step RT-PCR Kit from NEB (cat. #E5315 S). Both the OneTaq One-Step RT-PCR Kit and Luna Universal One-step RT-qPCR Kit (cat. #E3005E) were used for FIG. 4. FIG. 5 was performed exclusively with the Luna Universal One-step RT-qPCR Kit. Buffer and enzyme were used according to manufacturer recommendations for 100 μL total volume. All reactions contained Tween-20 at a final concentration of 1% v/v, 500 nM final concentration of each amplification primer, and 100 GCE of synthetic dsDNA spike-in molecules. Synthetic RNA samples were added at 2 μL/reaction to achieve the appropriate number of viral particles. Thermocycling was performed using an Applied Biosystems Veriti Thermal Cycler with the following cycling programs shown in Table 2:

TABLE 2
Cycling programs

| | | |
|---|---|---|
| # of Cycles | QneTaq | Luna |

| | | | | |
|---|---|---|---|---|
| 1x | 48° C. | 20:00 | 55° C. | 20:00 |
| 1x | 94° C. | 1:00 | 95° C. | 1:00 |
| 40x | 94° C. | 0:20 | 95° C. | 0:20 |
| | 55° C. | 1:00 | 55° C. | 1:00 |
| | 68° C. | 1:00 | 60° C. | 1:00 |
| 1x | 68° C. | 5:00 | 68° C. | 5:00 |

Sanger Sequencing

Sanger sequencing was performed by Sequetech Corporation using the BigDye Terminator Cycle Sequencing Kit and capillary electrophoresis was performed using a Applied Biosystems 3730xl DNA Analyzer.

Data Analysis

For concordance calls, Sanger sequencing was analyzed using the following procedure. The primary base sequence based on automatic calling were aligned to the viral genome. If the aligned sequence matched the viral genomic sequence without any deletion, then the sample is called positive for viral RNA. If the sequence does not match the reference, then the signal to noise ratio is checked with any less than 500 indicating insufficient signal which returns an indeterminate result. If signal to noise is greater than 500, then the ratio of genomic sequence to spike-in sequence is quantified by performing robust linear regression of genomic peak heights to spike-in peak heights.

Quantitation for FIG. 3 was performed with the same quality check as above but quantifying the terminal 6 bases of reference and spike-in sequence for all primary sequences, regardless of whether genomic, spike-in, or mixed sequence dominates. All analysis was performed using custom scripts in R, employing the seqinr and tidyverse packages.

### 6.2. Example 2: qSanger-COVID-19 Assay

The qSanger-COVID-19 Assay is a Sanger sequencing-based test for detection of SARS-CoV-2 RNA. The SARS-CoV-2 sequences and a spike-in sequence serving as an internal control are amplified with a primer pair designed to detect RNA from SARS-CoV-2 in upper respiratory swab specimens collected from patients who are suspected of COVID-19. Instruments employed to perform the test from sample collection to result include a thermal cycler (e.g. Applied Biosystems Veriti Thermal Cycler) and Sanger sequencing instrument (e.g. Applied Biosystems 3730xl DNA Analyzer).

6.2.1. Sample Collection

Patient samples is collected according to appropriate laboratory guidelines. All testing for COVID-19 is conducted in consultation with a healthcare provider. CDC guidelines for sample collection of upper respiratory swab specimens and sample storage is recommended.

Specimens are processed within 48 hours from collection and stored at 2-25° C. during that time as per the manufacturer's instructions. If the specimen is not tested within 48 hours samples should be stored frozen at −70° C. or colder.

Upper respiratory swab Collection. Once the swabs are collected as per the CDC guidelines above, it is recommended to use Universal Transport Medium (UTM) System for transportation and storage of swabs.

The qSanger-COVID-19 Assay does not require RNA extraction for normal assay performance. VTM from NP/OP swabs can be added directly to the reactions.

6.2.2. Amplification

1. Obtain and label a PCR plate for PCR Amplification.

2. Carefully clean the workspace with RNAse Away.

3. Remove reagents from −20° C. storage and allow to thaw on ice.

Briefly vortex the Reagent A1 (Primer and Spike-in Mix) tube and centrifuge to collect liquid. Return to ice for reaction assembly. Do not vortex the Luna reagents. Invert Enzyme A3 (Luna® Universal Probe One-Step Reaction Mix) and Enzyme A2 (NEB Luna® RT Enzyme) tubes to mix. Briefly spin down the tube and centrifuge to collect liquid. Return to ice for reaction assembly.

4. In an RNase-free conical tube, combine the following reagents at the listed volumes to prepare the Assembled Reaction Master Mix. Invert the tube to mix well and briefly centrifuge to collect liquid. Note: calculated volumes account for 10% excess for pipetting error.

TABLE 3
Assembled Reaction Master mix
Assembled Reaction Master Mix

| | | Volume (μL) |
|---|---|---|
| | | For full 96- |
| | Volume (μL) | well plate |
| Reagent | For N unknown samples | (N = 93) |
| Enzyme A3 (Luna ® Universal | =12.5*(N + 3)*1.1 | 1320 |
| Probe One-Step Reaction Mix) | | |
| Reagent A1 (Primer and Spike | =5*(N + 3)*1.1 | 528 |
| in Mix) | | |
| Enzyme A2 (NEB Luna RT | =1.25*(N + 3)*1.1 | 132 |
| Enzyme | | |
| Total | =18.75*(N + 3)*1.1 | 1980 |

5. Add 18.75 μL Assembled Master Mix to each well to be tested.

Tip: Use a reagent trough and a multichannel pipette to fill the PCR Amplification Plate.

6. Add 6.25 μL of control sample to each appropriate well, as detailed below. Gently pipette up and down to mix.

A01: Positive Control

B01: RNase-free water

7. Add 12.5 μL of Enzyme A3, 5 μL of Reagent C1, 1.25 μL of Enzyme A2, and 6.25 μL of RNase-free water to the well C01 for the no-template, no-spike-in control.

8. Add 6.25 μL unknown sample to each remaining well. Gently pipette up and down to mix.

9. Carefully apply plate seal to the PCR Amplification Plate such that it is airtight. Press each well to make sure it is sealed.

10. Briefly spin down the PCR Amplification Plate using the short spin feature on a plate centrifuge.

11. Load the PCR Amplification Plate on the thermal cycler and start the PCR Amplification Program.

12. Add the following PCR Amplification program to the thermal cycler. Ensure a heated lid is used.

6.2.3. PCR Clean-Up, Using ExoSAP

Prepare Reagents and Instrument

1. Obtain and label a PCR plate for PCR Clean-up.

2. Obtain reagents from −20° C. storage. Flick and invert tube to mix. Briefly spin down to collect liquid.

3. Obtain the PCR Amplification plate containing PCR Amplification products. Ensure reaction wells are well mixed. Briefly spin down with plate centrifuge to collect liquid.

4. Add the following PCR Clean-up program to the thermal cycler. Ensure a heated lid is used.

TABLE 4
PCR Clean-up Program
PCR Clean-Up Program

| Temperature | Time (mm:ss) | Cycles |
|---|---|---|
| 37° C. | 15:00 | 1x |
| 80° C. | 15:00 | |
| 4° C. | ∞ | |

Assemble and Run PCR-Clean Up Reaction

1. In a labeled 1.5 mL microcentrifuge tube, combine the following reagents at the listed volumes to prepare the ExoSAP Master Mix. Invert the tube to mix well and briefly centrifuge to collect liquid. Note: calculated volumes account for 10% excess for pipetting error.

TABLE 5
ExoSAP Master Mix
ExoSAP Master Mix (Full 96-well Plate)

| | Volume (μL) | Volume (μL) |
|---|---|---|
| | For n PCR Amplified | For full 96-well plate |
| Reagent | samples | (n = 96) |
| Nuclease-free water | =3 n*1.1 | 316.8 |
| ExoSAP-IT ™ PCR | =2*n*1.1 | 211.2 |
| Product Cleanup Reagent | | |
| Total | =5*n | 528 |

2. Add 5 μL of ExoSAP Master Mix to each appropriate well.

Tip: Use a reagent trough and a multichannel pipette to fill the PCR Clean-up Plate.

3. Add 2 μL of PCR Amplification product to each appropriate well.

Tip: Use a multichannel pipette to fill the PCR Clean-up Plate.

4. Carefully apply plate seal to the PCR Clean-up Plate such that it is airtight. Press each well to make sure it is sealed.

5. Briefly spin down the PCR Clean-up Plate using the short spin feature on a plate centrifuge.

6. Load the PCR Clean-up Plate on the thermal cycler and start the PCR Clean-up Program.

6.2.4. Cycle Sequencing, Using BigDye v3.1 Kit

Prepare Reagents and Instrument for Sequencing Reaction

1. Obtain and label a PCR plate for Cycle Sequencing.

2. Obtain reagents from −20° C. storage. Flick and invert tube to mix. Briefly spin down to collect liquid.

3. Obtain PCR Clean-up Plate containing PCR Clean-up products. Ensure reaction wells are well mixed. Briefly spin down with plate centrifuge to collect liquid.

4. Add the following Cycle Sequencing program to the thermal cycler. Ensure a heated lid is used.

Important note: Adjust the ramp rate such that it is <1° C./sec during cycling steps.

TABLE 6
Cycle Sequencing Program

| Temperature | Time (mm:ss) | Ramp Rate* | Cycles |
|---|---|---|---|
| 96° C. | 1:00 | 100%* | 1x |
| 96° C. | 0:10 | 37%* | 30x |
| 50° C. | 0:05 | 42%* | |
| 60° C. | 4:00 | 37%* | |
| 60° C. | 4:00 | 100%* | 1x |
| 4° C. | ∞ | 100%* | |

*Ramp rate setting used for validation runs on the Veriti Thermal Cycler

Assemble and Run Sequencing Reaction

1. In a labeled 1.5 mL microcentrifuge tube, combine the following reagents at the listed volumes to prepare the Cycle Sequencing Master Mix. Invert the tube to mix well and briefly centrifuge to collect liquid.

Note: calculated volumes account for 10% excess for pipetting error.

TABLE 7
Cycle Sequencing Master Mix
Cycle Sequencing Master Mix (Full 96-well Plate)

| | Volume (μL) | Volume (μL) |
|---|---|---|
| | for n cycle | For full 96-well plate |
| Reagent | sequenced sample | (n = 96) |
| Nuclease-free water | =2*n*1.1 | 211.2 |
| Reagent B1 (Sanger | =1*n*1.1 | 105.6 |
| Sequencing Primer) | | |
| BigDye Ready Reaction | =2*n*1.1 | 211.2 |
| Mix | | |
| Total | =5*n*1.1 | 3960 |

2. Add 5 μL of Cycle Sequencing Master Mix to each appropriate well.

Tip: Use a reagent trough and a multichannel pipette to fill the Cycle Sequencing Plate.

3. Add 5 μL of PCR Clean-up Product from PCR Clean-up Plate to each appropriate well.

Tip: Use a multichannel pipette to fill the Cycle Sequencing Plate.

4. Carefully apply plate seal to the Cycle Sequencing Plate such that it is airtight. Press each well to make sure it is sealed.

5. Spin down Cycle Sequencing Plate using the short spin feature on a plate centrifuge.

6. Load the Cycle Sequencing Plate in the thermal cycler and start the Cycle Sequencing program.

6.2.5. Dye-Terminator Clean-Up, Using CleanSEQ Kit

Prepare Reagents and Instrument for the Dye Terminator Clean-Up

1. Obtain and label a PCR plate for Sanger Sequencing.

2. Obtain Hi-Di Formamide from −20° C. storage. Allow to thaw at room temperature. Briefly spin down to collect liquid.

3. Obtain CleanSEQ Beads from 4° C. storage. Vortex the bottle until the beads are well mixed and in suspension.

4. Obtain Cycle Sequencing Plate containing Cycle Sequencing products. Ensure reaction wells are well mixed. Briefly spin down with plate centrifuge to collect liquid. Add “+Clean-up” to the plate label.

5. Prepare Sanger Sequencing Plate by adding 10 μL of Hi-Di Formamide to sample wells. Add 20 μL of nuclease-free water to any remaining wells in the plate.

Tip: Preparation of the Sanger Sequencing Plate can be done while waiting for elution incubations to complete.

Assemble and Run Dye-Terminator Clean-Up reaction.

1. In a conical tube, combine V Water mL of water and VEthanol mL of absolute ethanol to create 85% Ethanol. Vortex to mix and briefly centrifuge to collect liquid.

TABLE 8
Run Dye Terminator Clean-Up reagents
85% Ethanol*

| Reagent | Volume (mL) |
|---|---|
| Nuclease-free Water | 4.2 |
| Absolute Ethanol | 23.8 |
| Total | 28.0 |

*Note:
the above quantity of 85% Ethanol is sufficient for 1 full 96-well plate. The 85% Ethanol should be made fresh for each reaction and used within 24 hrs of creation.

2. Pipette 10 μL of CleanSeq Beads to each reaction well in the Cycle Sequencing Plate.

3. Pipette 42 μL of 85% Ethanol into each well. Pipette up and down until well mixed.

4. Position Cycle Sequencing+Clean-up Plate on the magnetic bead plate. Incubate at room temperature for 3 minutes or until the solution is clear.

Important Note: Maintain Cycle Sequencing+Clean-up Plate on the magnetic bead plate for subsequent steps.

5. Remove supernatant using a pipette.

Tip: Use a multichannel pipette to remove the supernatant.

6. Perform Wash 1:

1. Add 100 μL of 85% Ethanol to each well. Incubate for 30 seconds.

Tip: Use a multichannel pipette to add 85% Ethanol to each well.

2. Remove supernatant using a pipette.

Tip: Use a multichannel pipette to remove the supernatant from each well.

7. Perform Wash 2:

1. Add 100 μL of 85% Ethanol to each well. Incubate for 30 seconds.

Tip: Use a multichannel pipette to add 85% Ethanol to each well.

2. Remove supernatant using a pipette.

Tip: Use a multichannel pipette to remove the supernatant from each well.

8. Incubate at room temperature for 10 minutes or until dry.

Tip: Prepare the Sanger Sequencing Plate while waiting for elution incubations to complete.

9. Add 40 μL of nuclease free water to the sample. Pipet up and down to mix. Incubate at room temperature for 5 minutes to elute the sample.

Tip: Prepare the Sanger Sequencing Plate while waiting for elution incubations to complete.

10. Transfer 10 μL of eluted samples to the appropriate wells in the Sanger Sequencing plate.

11. Carefully apply plate seals to the Cycle Sequencing+Clean-up and Sanger Sequencing plates such that they are airtight. Press each well to make sure it is sealed.

6.2.6. Capillary Electrophoresis

Set-Up

1. Add COVID-19 qSanger run module.

2. Add COVID-19 qSanger analysis settings.

3. For each run, create a new Sequencing Analysis Plate Record.

a. Open the 3730xl Data Collection Software. Navigate to the Plate Manager.

b. Create a New Plate then complete the Sequencing Analysis Plate record by inputting the appropriate number of samples and selecting the COVID-19 Instrument and Analysis Protocols.

c. Add desired plates to the Run Scheduler.

4. Ensure that the DNA Analyzer is ready for a run. Complete all required maintenance activities prior to loading.

Protocol

1. Obtain Sanger Sequencing Plate containing Cycle Sequencing products that have undergone Dye-Terminator Clean-up. Centrifuge at 1000 g for 1 minute in a plate centrifuge. Ensure that no large bubbles are present in any of the wells.

2. Remove plate seal from Sanger Sequencing Plate and replace with a plate septa. Prepare 3730xl plate assembly by placing the septa-capped plate into a plate retainer.

3. Load the plate assembly onto the 3730xl instrument and begin the scheduled run.

6.2.7. Assessment of qSanger Results

Open ab1 files and inspect for the presence and absence of spike-in and native SARS-CoV-2 RNA sequences.

Processed ab1 files should include chromatograms clear of noise and show base calls for the entire length of the amplicon. Examples of types of traces for controls is shown in FIGS. 6-8.

6.2.8. Analysis of Sample Results

Positive Results

The electropherogram for positive samples depends on the relative amount of viral RNA to spike-in DNA in the initial RT-PCR reaction.

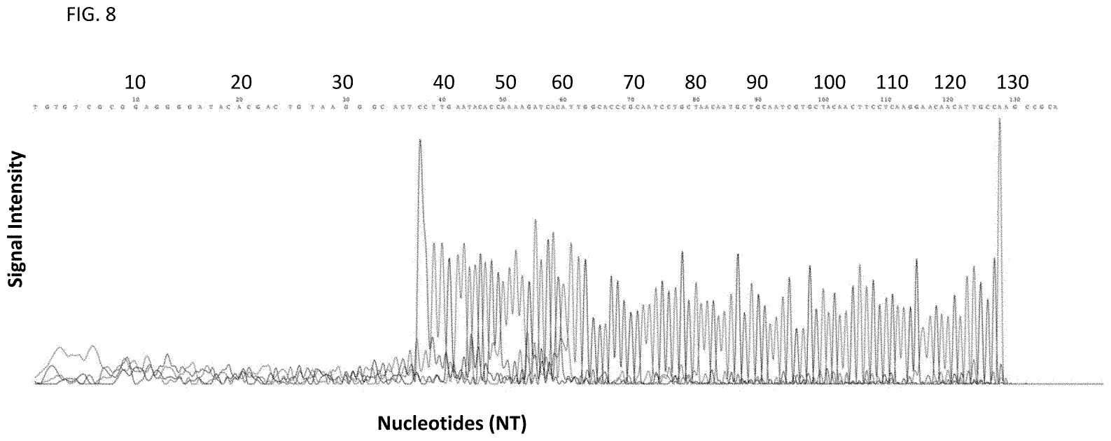

SARS-CoV-2 (COVID-19) RNA detected (strongly positive, see e.g. FIG. 8): Samples with relatively high viral RNA input result in electropherograms where the dominant signal is generated by the SARS-CoV-2 genomic sequence and the spike-in sequence is not visible.

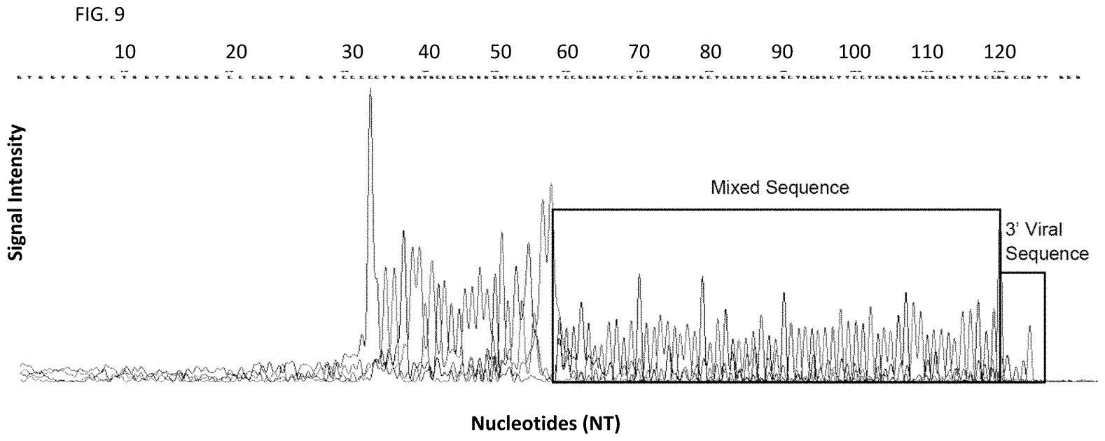

SARS-CoV-2 (COVID-19) RNA detected (weakly positive, see e.g., FIG. 9):

Samples with moderate or relatively low concentrations of viral RNA result in electropherograms where both the endogenous and spike-in sequence are visible. The example shown in FIG. 9 had relatively low RNA input, resulting in spike-in signal that is greater than viral sequence signal. The signal from the viral sequence, which is a longer genomic product, is visible at the 3′ end as well as in the mixed signal for the overlapping sequence. Note that the “PCR stop” setting on the sequencing instrument is turned off to obtain this data. Consequently, the base-caller may continue to make calls even if the sequence completely ends, so the base-calls at the 3′ end that are indicated at the top of the chromatogram may not provide meaningful data. The chromatogram itself should be inspected for the repeat sequence that would indicate the presence of viral SARS-CoV-2 RNA.

Negative Results

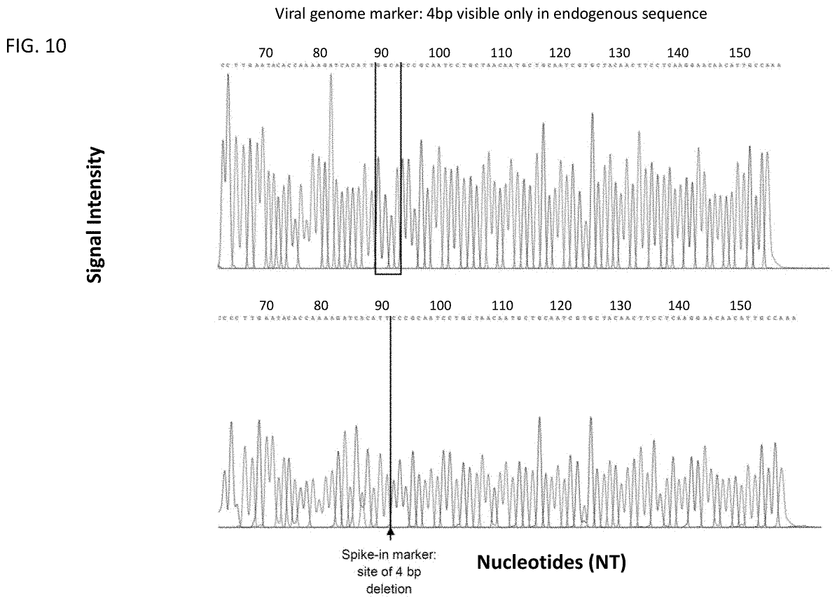

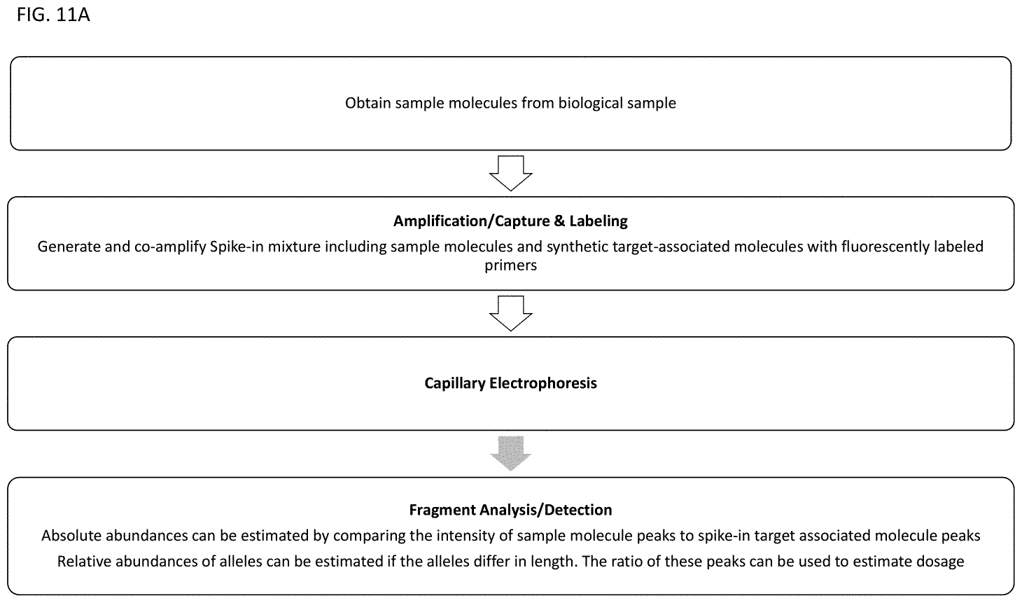

In negative samples, signal is produced only by the spike-in sequence. Negative samples show unmixed sequence matching the SARS-CoV-2 genome, differing by a 4 bp deletion. A comparison of highly positive (top image) vs. negative (spike-in only, lower image) electropherograms can be seen in FIG. 10.

6.2.9. Result Interpretation

a. qSanger-COVID-19 Assay Controls—Positive, Negative and Internal

All test controls should be examined prior to interpretation of patient results. If the controls are not valid, the patient results cannot be interpreted. Specifically, if the positive control is negative or invalid, the whole batch is reported as “INVALID”. If either of the two negative controls (No-RNA negative control or No-Template—No Spike-in control) is positive or invalid, the whole batch is reported as “INVALID”.

TABLE 9
Expected performance of AccuPlex reference materials
and negative and positive controls when valid

Expected Values

| | SARS-CoV-2 | Spike-In |
|---|---|---|
| Control | (N) | (IC) |
| No-RNA negative control | − | + |
| Positive Control: | + | + |
| Accuplex SARS-CoV-2 | | |
| Positive Reference | | |
| Material | | |
| No Template - no Spike- | − | − |
| in Control | | |

If any of the above controls do not exhibit the expected performance as described, the assay may have been improperly set up and/or executed improperly, or reagent or equipment malfunction could have occurred. Invalidate the run and re-test.

If all controls have the expected results, the patient specimens will be reported out as “POSITIVE” when SARS-CoV-2 alignment is detected, “NEGATIVE” when only spike-in alignment is detected, or “INVALID” when neither SARS-CoV-2 nor spike-in alignment is detected (assay failure) or when signal-to-noise in chromatogram QC is not sufficiently high (sequencing failure). “INVALID” specimens are retested once. If the retest result remains “INVALID”, then specimen recollection is recommended.

b. Examination and Interpretation of Patient Specimen Results:

Assessment of clinical specimen test results should be performed after the positive and negative controls have been examined and determined to be valid and acceptable. If the controls are not valid, the patient results cannot be interpreted.

TABLE 10
Patient Specimen Result Interpretation

| SARS- | Spike- | | |
|---|---|---|---|
| CoV-2 | In | Result | |
| (N) | (IC) | Interpretation | Patient Report Verbiage |
| + | −/+* | SARS-CoV-2 | SARS-coV-2 RNA detected |
| | | RNA detected | |
| − | + | SARS-CoV-2 | SARS-CoV-2 RNA NOT |
| | | RNA NOT | detected. |
| | | detected. | Negative results do not preclude |
| | | | SARS-CoV-2 (COVID-19) |
| | | | infection and should not be used |
| | | | as the sole basis for treatment |
| | | | or other patient management |
| | | | decisions. |
| − | − | Results are invalid. | Invalid |
| | | Repeat testing if the | This specimen resulted in assay |
| | | result is still invalid, | failure. The specimen might |
| | | a new specimen | have contained an inadequate |
| | | should be obtained. | amount of clinical material. |
| | | | Repeat testing if required with |
| | | | a newly collected specimen |

*The absence of the internal spike-in control is acceptable in positive samples because under high viral load (i.e. >5000 molecules/reaction), the signal for viral RNA is so much greater than that for the spike-in internal control, that the control may not be readily observed. This is expected behavior and does not indicate any assay failure as long as there is observable viral sequence.

The sequencing results must be manually inspected by trained personnel, to see if they align to both the spike-in and SARS-CoV-2 sequences. If the Sanger sequencing chromatogram aligns to the SARS-CoV-2 sequence alone, then this indicates that SARS-CoV-2 RNA was abundant in much higher level than the spike-in, and a POSITIVE result should be returned. If both SARS-CoV-2 and spike-in sequence alignments are found (mixed sequence), then SARS-CoV-2 RNA was present in the specimen at a comparable abundance to the spike-in, and as before, a POSITIVE result should be returned (weakly positive). If the spike-in alignment is recovered without a SARS-CoV-2 alignment (no final 4 bp tail), then SARS-CoV-2 RNA was not detected by the assay, and a NEGATIVE result is returned. If both spike-in and SARS-CoV-2 alignments are missing, then an assay failure occurred, and an INVALID result is returned.

6.2.10. Analytic Sensitivity and Limit of Detection (LOD)

The limit of detection was evaluated by spiking the Accuplex SARS-CoV-2 material (Seracare) into a pool of SARS-CoV-2 negative clinical NP swab matrix. The negative NP-swab pool was made from samples collected from individuals confirmed SARS-CoV-2 negative and were collected in viral transport media (VTM, Becton-Dickinson Viral Transport). A dilution series ranging from 50 copies/reaction (8000 copies/mL) to 4 copies/reaction (640 copies/mL) was prepared. Each concentration was tested with 20 replicates. The LOD was determined as the lowest concentration where the percentage of detected samples was 95% or above (Table 10). The LoD of the qSanger-COVID-19 Assay is 3200 copies/ml of sample.

| Accuplex | Copies/μL | Detected/ | |
|---|---|---|---|
| Copies/Reaction | of Sample | Tested | % Detected |
| 50 | 8 | 20/20 | 100% |
| 20 | 3.2 | 19/20 | 95% |
| 10 | 1.6 | 14/20 | 70% |
| 4 | 0.64 | 10/40 | 50% |

6.2.11. 11.2. Inclusivity

Spike-in sequences were designed using the viral genomic region approximately corresponding to the N3 region amplified by the CDC published N3 primer and probe sets. Spike-in molecules have sequences identical to SARS-CoV-2 sequence (LC528232) including base positions 28216 to 29280 but lacking 4 bases 28715-28718, in order to create a frameshift that can be detected in data analysis. Primers that co-amplify both SARS-CoV-2 and spike-in were used for amplification. Sequencing was performed using a nested forward primer to increase specificity in human specimens.

An in silico analysis of the test's primer binding sequences was performed with 4635 SARS-CoV-2 full-length sequences deposited in NCBI. Of these sequences, more than 99% of sequences are identical to the reverse primer and 98.5% are identical to the forward primer. 1.4% of sequences exhibit single SNPs in at position 5 from the 5′ end of the forward primer, accounting for a homology of 95.5%. Given the location of this SNP and the limited impact on melting temperature of the primers, it is anticipated that this SARS-CoV-2 sequence would still be detected by this assay. 98.5% of the sequences have predicted melting temperatures greater than or equal to the annealing temperature of the thermocycling reaction.

6.2.12. Cross-Reactivity

An in silico analysis of the test primer sequences was performed with the following organisms as shown in Table 11 below.

TABLE 11
Test Primer Sequence Data

| | | F Primer % | | R Primer % | |
|---|---|---|---|---|---|
| | | Homology | | Homology | |
| Accession | Description | (n/total bases) | | (n/total bases) | |
| NC_002645.1 | Human coronavirus 229E, complete genome | 55% | (12/22) | 52% | (12/23) |
| NC_006213.1 | Human coronavirus OC43 strain ATCC VR- | 41% | (9/22) | 43% | (10/23) |
| | 759, complete genome | | | | |
| NC_006577.2 | Human coronavirus HKU1, complete genome | 45% | (10/22) | 48% | (11/23) |
| NC_005831.2 | Human Coronavirus NL63, complete genome | 45% | (10/22) | 52% | (12/23) |
| NC_004718.3 | SARS coronavirus Tor2, complete genome | 91% | (20/22) | 100% | (23/23) |
| NC_019843.3 | Middle East respiratory syndrome | 64% | (14/22) | 43% | (10/23) |
| | coronavirus, complete genome | | | | |
| AC_000017.1 | Human adenovirus type 1, complete genome | 41% | (9/22) | 39% | (9/23) |
| NC_039199.1 | Human metapneumovirus isolate 00-1, | 50% | (11/22) | 39% | (9/23) |
| | complete genome | | | | |
| NC_003461.1 | Human parainfluenza virus 1, complete | 41% | (9/22) | 39% | (9/23) |
| | genome | | | | |
| NC_003443.1 | Human rubulavirus 2, complete genome | 41% | (9/22) | 39% | (9/23) |
| NC_001796.2 | Human parainfluenza virus 3, complete | 41% | (9/22) | 52% | (12/23) |
| | genome | | | | |
| NC_021928.1 | Human parainfluenza virus 4a viral cRNA, | 36% | (8/22) | 43% | (10/23) |
| | complete genome, strain: M-25 | | | | |
| NC_026423.1 | Influenza A virus | 50% | (11/22) | 52% | (12/23) |
| | (A/Shanghai/02/2013(H7N9)) segment 2 | | | | |
| | polymerase PB1 (PB1) and PB1-F2 protein | | | | |
| | (PB1-F2) genes, complete cds | | | | |
| NC_002204.1 | Influenza B virus RNA 1, complete sequence | 36% | (8/22) | 39% | (9/23) |
| NC_006309.2, | Influenza C virus (C/Ann Arbor/1/50) (all | 45% | (10/22) | 43% | (10/23) |
| NC_006308.2 | accessions) | | | | |
| NC_038308.1 | Human enterovirus 68 strain Fermon, | 41% | (9/22) | 39% | (9/23) |
| | complete genome | | | | |
| NC_001803.1 | Respiratory syncytial virus, complete genome | 50% | (11/22) | 39% | (9/23) |
| NC_009996.1 | Human rhinovirus C, complete genome | 41% | (9/22) | 43% | (10/23) |
| NC_005043.1 | Chlamydia pneumoniae TW-183, complete | 50% | (11/22) | 57% | (13/23) |
| | sequence | | | | |
| NZ_QQLA01000002.1/ | Haemophilus influenzae strain M14791 | 59% | (13/22) | 57% | (13/23) |
| NZ_MZJN01000009.1 | M14791_HUY4654A129_cleaned_ctg_921, | | | | |
| | whole genome shotgun | | | | |
| | sequence/Haemophilus influenzae strain | | | | |
| | 48P45H1 N48P45H1_11_8, whole genome | | | | |
| | shotgun sequence | | | | |
| NZ_QFHP01000013.1/ | Legionella pneumophila strain HH56 | 64% | (14/22) | 61% | (14/23) |
| NZ_QFHP01000039.1 | NODE_13_length_107514_cov_50.6576, | | | | |
| | whole genome shotgun sequence/Legionella | | | | |
| | pneumophila strain HH56 | | | | |
| | NODE_39_length_871_cov_120.831, whole | | | | |
| | genome shotgun sequence | | | | |
| No Similarity | Mycobacterium tuberculosis (taxid: 1773) | NA | | NA | |
| Found | | | | | |
| (taxid: 1773) | | | | | |
| NZ_CGVP01000016.1 | Streptococcus pneumoniae strain SMRU22, | 64% | (14/22) | 65% | (15/23) |
| | whole genome shotgun sequence | | | | |
| NZ_CAAINE010000002.1/ | Streptococcus pyogenes strain NS678, whole | 64% | (14/22) | 61% | (14/23) |
| NZ_CAAHYZ010000009.1 | genome shotgun sequence/Streptococcus | | | | |
| | pyogenes strain 31089V2S1, whole genome | | | | |
| | shotgun sequence | | | | |
| NZ_CSNY01000165.1 | Bordetella pertussis strain B082 isolate | 55% | (12/22) | 57% | (13/23) |
| | 1977/3, whole genome shotgun sequence | | | | |
| NZ_BLHG01000007.1 | Mycoplasma pneumoniae strain KPI-131 | 50% | (11/22) | 52% | (12/23) |
| | contig_7, whole genome shotgun sequence | | | | |
| NW_017264788.1 | Pneumocystis jirovecii RU7 chromosome | 59% | (13/22) | 91% | (20/22) |
| | Unknown supercont1.14, whole genome | | | | |
| | shotgun sequence | | | | |
| NC_032090.1/ | Candida albicans SC5314 (all accessions) | 68% | (15/22) | 61% | (14/23) |
| NC_032096.1 | | | | | |
| NZ_CAADQY010000466.1 | Pseudomonas aeruginosa isolate XDR-PA, | 68% | (15/22) | 65% | (15/23) |
| | whole genome shotgun sequence | | | | |
| NZ_CP035288.1 | Staphylococcus epidermidis strain ATCC | 59% | (13/22) | 57% | (13/23) |
| | 14990 chromosome, complete genome | | | | |
| NZ_PKHZ01000004.1/ | Streptococcus salivarius strain | 59% | (13/22) | 70% | (16/23) |
| NZ_WMYP01000001.1 | UMB0028.21837_8_51.4, whole genome shotgun | | | | |
| | sequence/Streptococcus salivarius strain | | | | |
| | BIOML-A3 scaffold1_size599083, whole | | | | |
| | genome shotgun sequence | | | | |
| NC_001897.1 | Human parechovirus, genome | 45% | (10/22) | 43% | (10/23) |
| NZ_BEYJ01000010.1/ | Staphylococcus aureus strain GUATP 151, | 64% | (14/22) | 65% | (15/23) |
| NZ_PSZX01000019.1 | whole genome shotgun sequence/ | | | | |
| | Staphylococcus aureus strain | | | | |
| | SKY9-1 SKY9-1_R1_(paired)_contig_19, | | | | |
| | whole genome shotgun sequence | | | | |
| NC_007530.2 | Bacillus anthracis str. ‘Ames Ancestor’, | 55% | (12/22) | 61% | (14/23) |
| | complete sequence | | | | |
| NC_014147.1 | Moraxella catarrhalis BBH18, complete | 55% | (12/22) | 57% | (13/23) |
| | genome | | | | |
| NZ_CP031252.1 | Neisseria elongata strain M15911 | 64% | (14/22) | 52% | (12/23) |
| | chromosome, complete genome | | | | |
| NZ_OAAT01000028.1/ | Neisseria meningitidis strain Neisseria | 64% | (14/22) | 65% | (15/23) |
| NZ_OAAT01000003.1 | meningitidis isolate R575, whole genome | | | | |
| | shotgun sequence | | | | |
| NZRQHK01000017.1/ | Leptospira sp. (all accessions: taxid: 171) | 64% | (14/22) | 74% | (17/23) |
| NZ_NPEI01000001.1 | | | | | |
| NC_017287.1 | Chlamydia psittaci 6BC, complete sequence | 55% | (12/22) | 70% | (16/23) |

Among the tested organisms, only SARS-coronavirus (SARS-CoV) exhibited more than 80% homology for the primer sequences. The forward primer had 91% homology (corresponding to 2 mismatches) and the reverse primer exhibited 100% homology.

However, because this assay sequences the internal sequence of the amplicon, the assay is able to distinguish SARS-CoV from SARS-CoV-2 based on the 4 SNPs in the internal control sequence that differentiate these two sequences. Moreover, as SARS-coronavirus is not currently circulating in the population, any cross-reactivity is not expected to result in false positives.

BLAST analysis indicated all other species had less than 80% homology in both amplification primers with the exception of Pneumocystis jirovecii which exhibited 91% homology in the reverse primer but only 59% homology in the forward primer. These primers are separated by 20,000 base-pairs and as such extremely unlikely to result in an amplification product that could also be sequenced by the qSanger-COVID-19 Assay. In addition, the intervening sequence is not homologous to SARS-CoV-2 and because the test is sequencing based and thereby identifies the specific organism, no false positive result would be generated.

6.2.13. Interfering Substances

The qSanger-COVID-19 Assay does not require RNA purification and therefore, potentially interfering substances commonly found in NP swab samples were tested for potential interference. The indicated final concentration of each substance was added to pooled negative clinical sample matrix and samples were tested in the absence and presence of 2× LoD of Accuplex SARS-CoV-2 material (Seracare). All conditions were tested in triplicate and the results analyzed for detection of COVID19. Results are summarized below.

TABLE 12
Interfering Substances only

Detected/Tested

| Substance | Concentration | | Negative | Positive |
|---|---|---|---|---|
| Afrin | 10% | v/v | 0.3 | 3/3 |
| Blood | 5% | v/v | 0.3 | 3/3 |
| Capacol | 5 | mg/mL | 0.3 | 3/3 |
| Flonase | 5% | v/v | 0.3 | 3/3 |
| Mucin | 2.5 | mg/mL | 0.3 | 3/3 |
| Mupirocin | 5 | mg/mL | 0.3 | 3/3 |
| Tamiflu | 2.2 | μg/mL | 0.3 | 3/3 |
| Tobramycin | 4 | μg/mL | 0.3 | 3/3 |

| | | | |
|---|---|---|---|
| Matrix control | NA | 0.3 | 3/3 |

6.2.14. Clinical Evaluation

For the clinical validation, 30 SARS-CoV-2 positive Nasopharyngeal Swabs and 30 SARS-CoV-2 negative Nasopharyngeal Swabs collected in Becton Dickinson Universal Viral Transport (specifically these Catalog #'s BD 220527, 220529 and 220531) were tested. The samples were collected during standard clinical visits at an academic medical center and had prior SARS-CoV-2 RT-PCR results obtained with EUA authorized RT-PCR tests.

The qSanger-COVID-19 Assay for clinical validation was performed. Raw data was analyzed, and outcome determined. Positive Percent Agreement (PPA) and Negative Percent Agreement (NPA) were calculated in comparison to the prior result with the FDA authorized test.

TABLE 13
Clinical Validation

| | |
|---|---|
| NP Swabs | EUA authorized |
| qSanger- COVID-19 | Comparator Assays |

| Assay | | Positive | Negative | Total |
|---|---|---|---|---|
| qSanger | Positive | 27 | 0 | 27 |
| COVID-19 | Negative | 3* | 30** | 33 |
| Assay | | | | |

| | | | |
|---|---|---|---|
| Total | 30 | 30 | 60 |

*The missed samples were retested on an additional EUA-authorized test and had the following Ct values with that test: for the S-Gene: 31.8, 31.3, and 33.0; the N-gene: 29.3, 30.8, and 33.0; and ORF1Ab: 29.7, 29.3, and 29.6. The mean Cts of this additional EUA authorized test at its LoD for NP swab are 34.3 (S), 29.1 (N) and 30.7 (ORF1ab), indicating that all three samples were low positive samples and therefore likely below the LoD of the qSanger-COVID-19 Assay.
**4 out of 30 samples had assay failure (no sequence present in .ab1 file, i.e., invalid results), and upon repeat, they all resulted in negative calls.

PPA: 27/30=90% (95% CI: 74.4-96.5%); NPA: 30/30=100% (95% CI: 88.7-100%).

6.2.15. Assay Troubleshooting

The qSanger-COVID-19 troubleshooting guide is divided into two main sections: RT-PCR and sequencing. These chemistries can be treated largely independently, however successful sequencing is often diagnostic of RT-PCR issues. Therefore, sequencing failures are discussed first, followed by RT-PCR failures and additional troubleshooting steps.

Identifying Sequencing Failures

Sanger sequencing data generated by the qSanger COVID-19 assay is useful for diagnosing assay failures.

Sequencing Failures

DNA Quantification

Perform DNA quantification on the samples to determine the concentration of DNA product in the reactions. A normal range is 10-25 ng/μL.

Methods: Qubit Fluorometer (Thermo Fisher), NanoDrop (Thermo Fisher), or similar

Gel Electrophoresis

Perform gel electrophoresis on the samples to determine the amplicon size(s) of the DNA products in the reactions.

Methods: traditional agarose gel electrophoresis, Tapestation (Agilent), or similar

### 6.3. Example 3: Fragment Analysis

Fragment analysis was proposed as an additional alternative approach for sanger sequencing for infectious disease read out. Fragment analysis is similar to Sanger in that it is run via capillary electrophoresis (CE) using the same DNA Analyzer instrument. The use of CE results in a measurable size separation of signals, meaning applying a qSanger-like analysis is theoretically possible. Rather than labeling each base as in Sanger, fragment analysis uses fluorescent end-point labeling wherein fluorescent dyes are attached to labeling primers and incorporated into samples through a PCR reaction. Fragment analysis allows for target molecules to be separated by both size and color space, meaning a single injection can generate data for many independent loci. Additionally, fragment analysis requires only two PCR reactions (amplification and labeling) and does not involve any bead purification as labeled product is directly diluted and denatured in formamide for injection, meaning that fragment analysis requires less operator time and likely has a reduced reagent cost as compared to qSanger.

Proof of Concept

Amplification

Two loci were selected for amplification, differing by size; targets were either 60 bp or 80 bp. Although 2 specific loci were chosen for the proof of concept, any DNA or RNA molecule targets specific to any infectious diseases described in the present disclosure will apply to the proof of concept described herein.

Primers included a universal tailed sequence to be used for labeling. Two primer mixes where tested: a singleplex reaction with the 60 bp primers alone and a multiplex reaction with both the 60 bp and 80 bp primers. Sample (gDNA) and spike-in (gBlock) inputs were approximately 1:1. Samples were amplified under standard PCR conditions at 30 cycles with 8 replicates per condition. Amplification products were pooled for downstream use to eliminate amplification noise.

Universal Labeling

Two different labeling methods were tested: high cycle count with low product input (30 cycles, 0.1 ng) and low cycle count with high product input (6 cycles, 20 ng). Labeling primer mixes included a FAM labeled primer and an unlabeled primer, which were complementary to the universal sequences included in the amplification primer tails. Two different labeling primer mixes were tested to assess the labeling of the forward (“FAMF”) and the reverse (“FAMR”) strands. Reactions were run at 16 replicates each and products were retained in individual wells to assess the labeling noise. A pool was also made from two labeling reaction products (60 bp labeled at 30 cycles with FAMF and 60 bp labeled at 6 cycles with FAMF) to assess the shot noise.

Injection

Labeled products were combined with size standard and diluted with formamide at the manufacturer's recommended volumetric ratios (1:1:18). The formamide-diluted samples were plated for injection at a final volume of 104, in a honeycomb arrangement to avoid using adjacent capillaries due to the risk of signal cross-talk. All samples were plated with a single replicate with exception to the pooled samples, which were plated at 24 replicates each. Injection plates were heat denatured using a thermal cycler and loaded onto the 3730xl DNA Analyzer. Default GeneMapper injection settings for fragment analysis were used with a reduced injection time and voltage to avoid signal saturation. Plates with standard unpooled samples were reinjected three times for a total of four injections. Pooled sample plates were injected only once.

Data Processing

Data was processed using the online Microsatellite Analysis Software by Thermo Fisher. Processing parameters were kept at the default settings, with exception to the minimum signal for the FAM (blue) color channel which was increased to 500 RFU to remove residual noise.

Results and Discussion

Base Composition Affects Measured Size

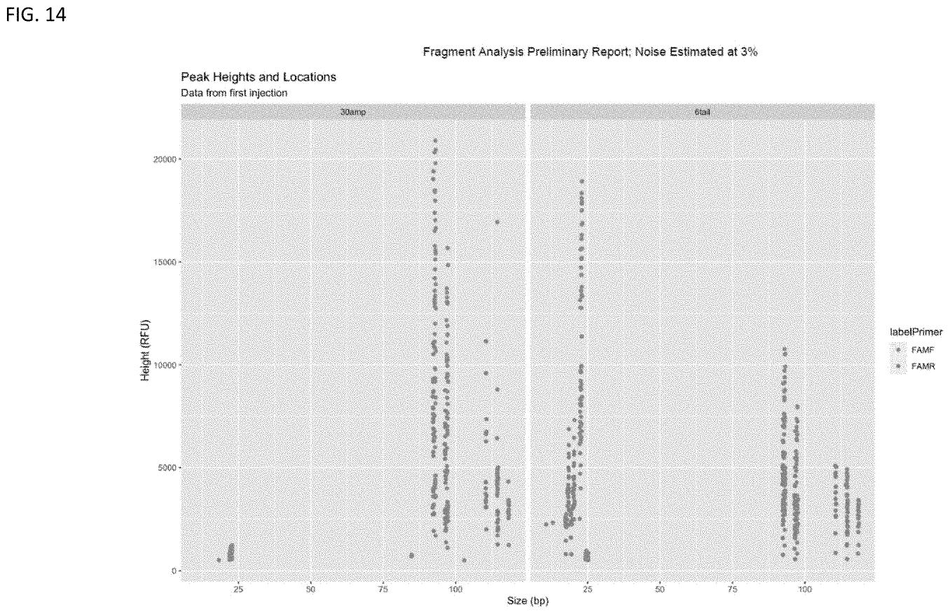

NA12878 gDNA samples were amplified with primers for either a 60 bp or a 60 bp and 80 bp amplicon and pooled prior to labeling. Pooled amplified samples were universally labeled using a primer set in which only one primer (either the forward “FAMF” or reverse “FAMR” primer) was labeled with a FAM fluorescent dye. Labeled samples were combined with a size standard, diluted 20× in formamide, heat denatured, and injected as single stranded DNA on the 3730xl DNA Analyzer. The resultant fsa files were processed using the online Microsatellite Analysis tool by Thermo Fisher to determine peak location and sizing. Processing parameters were kept at the default settings, with exception to the minimum signal for the FAM (blue) color channel which was increased to 500 RFU to remove residual noise. The peak data for the first injection with these described minimum height and size filters applied can be seen in FIG. 14.

FIG. 14 shows peaks in two main size groups: those less than 25 bp and those that are within the 90-120 bp range. The peaks at 25 bp or less are the result of residual unincorporated labeling primers, which are 20 bp each. The 6 cycle labeling method notably has a higher number of peaks measuring below 25 bp as compared to the 30 cycle labeling method. This means that the 6 cycle labeling approach has more residual labeling primers. For downstream analysis, peak data was filtered to remove peaks from the unincorporated primer.

Peaks measuring in the 90-120 bp range are from the labeled target molecules, which are designed to be 96-120 bp. For peaks within this range, the observed signal intensity varies per sample. Samples labeled with the 30 cycle method appeared to have higher maximum intensities than the 6 cycle labeling approach, which is expected given the trends in residual unincorporated labeling primer across these two methods. Note that the samples labeled with the FAMF primers appear to run at a higher molecular weight than those labeled with the FAMR primers, as can be seen particularly with the highest molecular weight peaks which correspond to the labeled 80 bp amplicon. The systematic difference in measured size is due to the varied base composition in the forward and reverse strands. Since labeled samples are denatured and run on capillary electrophoresis as ssDNA, molecular weight can vary for products of the same length.

Consecutive Peaks are Clearly Resolved

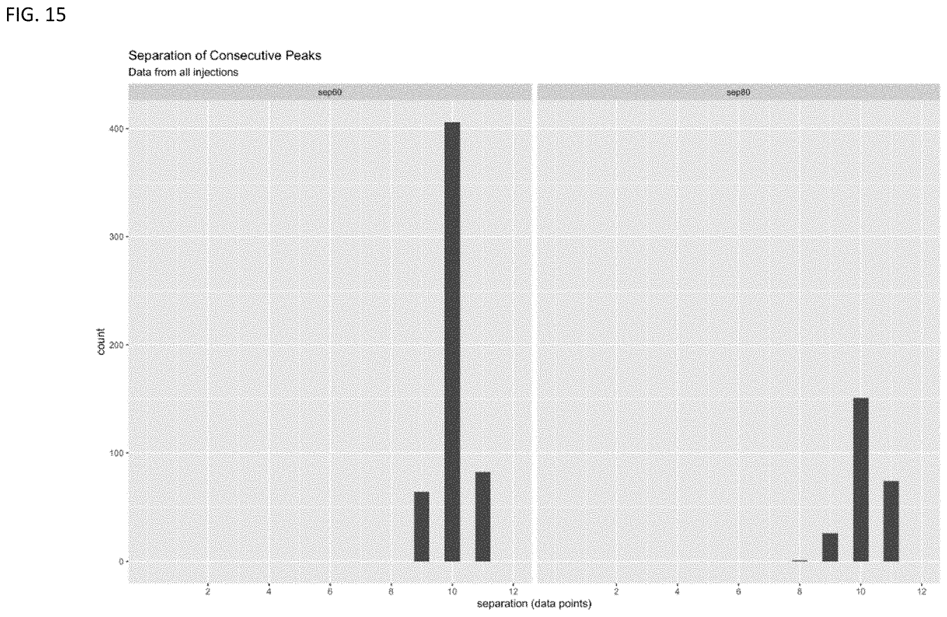

For all samples, peaks were distinguished and labeled by their size. Spike-ins differed from reference sequence by a 4 bp deletion, resulting in a staggered qSanger-like peak arrangement. The processed peak data outputs both the beginning and ending points of the detected peak, documented as data points or scan numbers where one base is approximately 7 data points. The difference in data points of consecutive peaks was calculated and can be seen in FIG. 15.

As shown in FIG. 15, consecutive peaks had a minimum separation of 8 data points, meaning none of the detected target peaks overlapped with another and a 4 bp deletion gives sufficient separation. The peaks are thus all clearly resolved and can be treated independently in downstream calculations.

Shot noise is around 2-2.5%

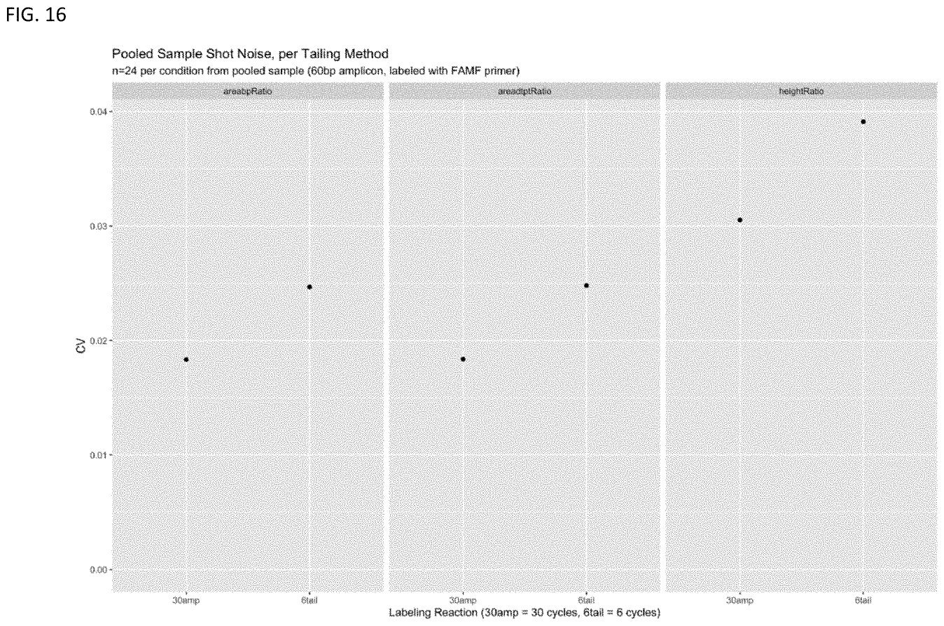

Shot noise was measured by injecting many technical replicates (n=24) on a single plate. Technical replicates were prepared by pooling products across all replicates of a labeling reaction condition. This pooled labeled product was combined with the size standard and diluted in formamide. The sample and size standard mixture was aliquotted into 24 wells of a plate for injection. The two different FAMF labeling reactions (30 cycle and 6 cycle) on the singleplex (60 bp amplicon) sample were used to assess the shot noise. The reference to spike-in ratios were calculated using three different peak values: peak area in base pairs, peak area in data points, and peak height. The CV for each of these reference to spike-in ratio types is shown in FIG. 16.

For the reference: spike-in ratios calculated by area, the CV is approximately 2-2.5%. The CV for the ratios calculated by peak height is higher, at 3-4%, suggesting that peak height is subject to more noise and is thus less indicative of fragment quantity than peak areas.

The two area types (base pairs and data points) did not appear to affect the observed noise. This is perhaps because base pairs are the calibrated version of data points. The calibration likely does not affect the observed noise because the assay read-out is an intrasample ratio of two peaks. The intrasample ratio itself serves as an internal control for any sample-to-sample variations that calibration would otherwise correct. To simplify downstream figures, only one area type (in base pairs) is displayed. Note that the 6 cycle labeling method appears to systematically have a higher shot noise than the 30 cycle method for all ratio type. This systematic difference suggests that shot noise is not constant but rather a function of parameters related to sample composition.

Labeling Techniques Vary in Absolute Intensity

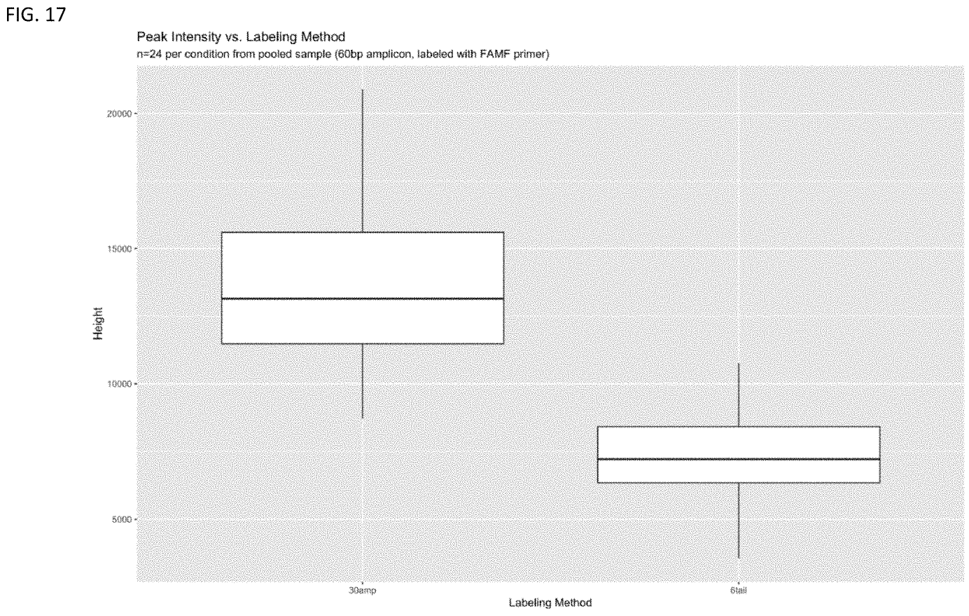

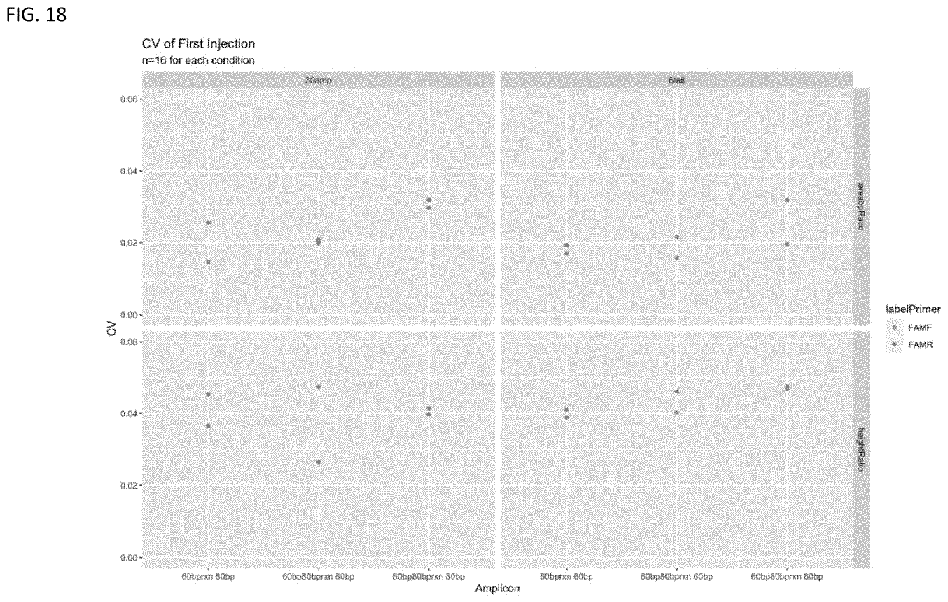

The higher shot noise in samples labeled using 6 cycles could potentially be explained by the difference in absolute intensities seen across the two labeling methods. The distribution of peak intensities for each labeling method is shown in FIG. 17.

The 30 cycle labeling method resulted in peaks of higher intensities as compared to the 6 cycle labeling method. If shot noise were a function of intensity, the systematically higher shot noise for lower signals would be explained. To confirm this hypothesis, and additional experiment would need to be run using a sample injected on a dilution gradient to control for sample composition.

Assay Noise is Around 2-3% for the First Injection

Assay noise was assessed using 16 replicates of samples labeled either with the forward or reverse FAM primer at 6 or 30 cycles. Labeling template consisted of amplified product containing amplicons of 60 bp or 60 bp and 80 bp in length. Amplified product was pooled prior to labeling to eliminate noise from the initial amplification reaction. Labeled products were combined with a size standard and denatured in formamide for injection. Reference to spike-in ratios were calculated using both area and height data for the detected peaks. The CV of the reference to spike-in ratio per tested condition is shown in FIG. 28.

For reference to spike-in ratios calculated using the peak area, the assay CV was estimated at around 2-3%. The assay CV for reference to spike-in ratios calculated using peak height was higher, at around 3-5%. All assay noise estimate values are very similar in value to the associated shot noise, meaning that shot noise likely dominates. Thus, using peak area to calculate the reference to spike-in ratio appears to be the best option to reduce noise.

The estimated assay noise was similar for the 60 bp amplicon across the two different amplification reactions. This means that adding an additional amplicon of a different size did not appear to significantly affect the CV.

For the data calculated using the peak area, there did not seem to be a major difference in assay noise between the two labeling reactions. Since the 30 cycle reaction had fewer residual labeling primers as seen in the “30 amp” of FIG. 28, meaning the labeling reaction conditions are closer to saturation than those in for the 6 cycle reaction. Reaching saturation in the labeling reaction could be useful if adding additional labeling primers of different color and sequence.

6.3.1. Conclusions

Fragment analysis is sensitive to molecular weights, since injected fragments are single stranded.

The 30-cycle labeling approach has less signal from residual labeling primers than the 6-cycle labeling method and thus is likely closer to saturation.

Consecutive peaks are clearly resolved for spike-ins containing a 4 bp deletion.

Shot noise is around 2-2.5% for ratios computed using peak area. Shot noise was higher (3-4%) for ratios computed using peak height.

Shot noise is lower for the 30-cycle labeling reaction as compared to the 6 cycle labeling reaction. This may be due to differences in absolute intensities.

Total assay noise is around 2-3% for ratios computed using peak area. Assay noise was higher (3-5%) for ratios computed using peak height. Thus, ratios should be computed using peak area to reduce noise.

For infectious disease application, fragment analysis can include multiplexed measurements to be taken from same sample to drive down the noise. Multiple measurements can theoretically be taken from the same injection if both color and size space is utilized:

### 6.4. Example 3: Fragment Analysis for Detecting Infectious Diseases

The use of fluorescently labeled primers to co-amplify a set of target associated DNA molecules corresponding to each pathogen that is tested with a sample containing an unknown amount of pathogen genome molecules is tested by preparing several suspensions of pathogen molecules (ranging from 0 to 10000 molecules/mL at logarithmic intervals).

A master mix of fluorescently labeled primers, pathogen-associated molecules (25 molecules), reverse transcriptase, and a DNA polymerase is prepared and pipetted into PCR tubes. To each tube, a sample of one of the suspensions of pathogen molecules is added. There is 20 tubes (replicates) at each concentration of suspended pathogen molecules. Each reaction should be amplified. Amplified samples are combined with formamide and size standard followed by injection using a capillary electrophoresis instrument.

The output data includes chromatograms containing peaks corresponding to the size each of the molecular species present in the co-amplified mixture. Samples containing pathogen molecules will have peak(s) appearing corresponding to those molecules. If no pathogen is present, peaks corresponding to each of the synthetic target-associated molecules should be present.

If the peaks corresponding to each of the synthetic target-associated molecules are not present and no pathogen peak is present, the reaction has not proceeded as expected and no result can be interpreted.

### 7. EQUIVALENTS AND INCORPORATION BY REFERENCE

While the (1) disclosure has been particularly shown and described with reference to a preferred embodiment and various alternate embodiments, it will be understood by persons skilled in the relevant art that various changes in form and details can be made therein without departing from the spirit and scope of the disclosure.

All referenced issued patents and patent applications cited within the body of the instant specification are hereby incorporated by reference in their entirety, for all purposes.

1. A method of detecting the presence or absence of a coronavirus in a sample obtained from a subject, the method comprising:
generating a spike-in mixture including sample molecules from the sample and synthetic target-associated molecules, wherein the synthetic target-associated molecules comprise:
a target-matching region having a nucleotide sequence that matches a corresponding nucleotide sequence in a first region of the coronavirus's nucleotide sequence; and
a target-variation region that is distinguishable from a second region of the coronavirus's nucleotide sequence, the target-variation region having a nucleotide sequence with an insertion or deletion as compared to a corresponding nucleotide sequence in the second region of the coronavirus's nucleotide sequence;
co-amplifying the spike-in mixture to generate a co-amplified spike-in mixture;
performing capillary electrophoresis on the co-amplified spike-in mixture to generate a chromatogram-related output comprising a plurality of chromatogram intensities, the intensities including one or more peaks, the one or more peaks including at least one of:
a peak associated with the synthetic target-associated molecules; or
a peak associated with the coronavirus nucleotide sequence; and
determining the presence or absence of the coronavirus based on the peaks, wherein a position of the peak associated with the synthetic target-associated molecules is offset as compared to an expected location of the peak associated with the coronavirus nucleotide sequence.

2. The method of claim 1, wherein generating a spike-in mixture including sample molecules from the sample and synthetic target-associated molecules comprises:
mixing the target-associated molecules with the sample molecules; and
performing reverse transcription on the spike-in mixture to convert the sample molecules into DNA.

3. The method of claim 1, wherein the method does not include RNA extraction of the sample molecules.

4. The method of claim 1, wherein the chromatogram-related output comprises alignment positions corresponding to the chromatogram intensities, wherein the chromatogram intensities comprise first peaks associated with:
the target-matching region of the synthetic target-associated molecules;
the target-variation region of the synthetic target-associated molecules; and
a region of the sample molecules of the subject that corresponds to the target-variation region of the synthetic target-associated molecules,
wherein, for each of the different pairs, the base of the nucleotide sequence of the synthetic target-associated molecule corresponds to a first alignment position that is different from a second alignment position corresponding to the base of the nucleotide sequence of the sample molecule, and wherein the alignment positions of the chromatogram-related output comprise the first and the second alignment positions.

5. The method of claim 1, wherein co-amplifying the spike-in mixture comprises amplifying the synthetic-target associated molecules and the sample molecules with a set of primers, wherein the set of primers include nucleotide sequences that are complementary or reverse complementary to the target matching region of the synthetic target-associated molecules and are complementary or reverse complementary to the first region of the coronavirus's nucleotide sequence.

6. (canceled)

7. (canceled)

8. (canceled)

9. (canceled)

10.

11. The method of claim 5, wherein the set of primers comprise forward and reverse primers and fluorescently labeled tags attached at the 5′ end of the primer sequences.

12. The method of claim 10, wherein the co-amplified mixture comprises synthetic target-associated amplicon products and, when coronavirus is present in the sample, coronavirus amplicon products, the synthetic target-associated amplicon products comprising a nucleotide length that is shorter or longer than a nucleotide length of the second region of the coronavirus's nucleotide sequence.

13. (canceled)

14. (canceled)

15. (canceled)

16. The method of claim 1, wherein each chromatogram peak comprises one or more peak intensities associated with at least one of:
the target-matching region of the synthetic target associated molecules;
the target variation region of the synthetic target associated molecules; or
the region of the coronavirus's nucleotide sequence that corresponds to the target-variation region of the synthetic target-associated molecules.

17. The method of claim 15, wherein:
the peak intensity of a region of the sample molecules that corresponds to the target-variation region of the synthetic target-associated molecules includes a peak intensity position that is offset as compared to a peak intensity position of the target-variation region, wherein the offset corresponds to the insertion or deletion of one or more nucleotides in the target-variation region; or
the peak intensity of the region of the sample molecules that corresponds to the target-variation region of the synthetic target-associated molecules includes a peak intensity position that is offset as compared to the peak intensity position of the target-variation region, wherein the peak intensity position is offset by a distance away from the peak intensity of the target-variation region of the synthetic target-associated molecule.

18. (canceled)

19. The method of claim 16, wherein the method further comprises determining an absolute abundance of coronavirus nucleotide molecules by comparing the peak intensities of the region of the coronavirus's nucleotide sequence that corresponds to the target-variation region of the synthetic target-associated molecules to the peak intensities of the target-variation region of the synthetic target-associated molecules, wherein the absolute abundance is determined based on a known number of synthetic target-associated molecules added to the sample spike-in mixture.

20. The method of claim 16, wherein the method further comprises calculating the ratio of peak intensities of the region of the coronavirus's nucleotide sequence that corresponds to the target-variation region of the synthetic target-associated molecules to the peak intensities of the target variation region of the synthetic target-associated molecules.

21. (canceled)

22. (canceled)

23. (canceled)

24. The method of claim 1, wherein the coronavirus is selected from the group consisting of: coronavirus OC43, coronavirus 229E, coronavirus NL63, coronavirus HKU1, middle east respiratory syndrome beta coronavirus (MERS-CoV), severe acute respiratory syndrome beta coronavirus (SARS-CoV), and SARS-CoV-2.

25. A method of detecting the presence or absence of one or more infectious diseases from a sample obtained from a subject, the method comprising:
generating a spike-in mixture including sample molecules from the sample and synthetic target-associated molecules, wherein the synthetic target-associated molecules comprise:
a target-matching region that matches a corresponding nucleotide sequence in a first region of the infectious disease's nucleotide sequence, and
a target-variation region that is distinguishable from a second region of the infectious disease's nucleotide sequence, the target-variation region having a nucleotide sequence with an insertion or deletion as compared to a corresponding nucleotide sequence in the second region of the infectious disease's nucleotide sequence;
co-amplifying the spike-in mixture to generate a co-amplified spike-in mixture;
performing capillary electrophoresis on the co-amplified spike-in mixture to generate a chromatogram-related output comprising a plurality of chromatogram intensities, the intensities including an intensity associated with:
the synthetic target-associated molecules; and
the sample molecules of the subject; and
determining the presence or absence of at least one infectious disease based on the chromatogram intensities associated with the synthetic target-associated molecules and the sample molecules.

26. The method of claim 24, wherein comparing the chromatogram intensities associated with the synthetic target-associated molecules and the sample molecules of the subject comprises comparing a peak intensity position associated with the synthetic target-associated molecules and a peak intensity position of the sample molecules of the subject, wherein the peak intensity position of the synthetic target-associated molecules is offset as compared to the peak intensity position of the sample molecules.

27. The method of claim 24, wherein said performing capillary electrophoresis on the co-amplified spike-in mixture comprises sanger sequencing the co-amplified spike-in mixture.

28. (canceled)

29. (canceled)

30. The method of claim of claim 24, wherein the chromatogram-related output comprises alignment positions corresponding to the chromatogram intensities, wherein the chromatogram intensities comprise peaks associated with:
the target-matching region of the synthetic target-associated molecules;
the target-variation region of the synthetic target-associated molecules; and
the second region of the infectious disease's nucleotide sequence,
wherein, for each of the different pairs, the base of the nucleotide sequence of the synthetic target-associated molecule corresponds to a first alignment position that is different from a second alignment position corresponding to the base of the nucleotide sequence of the sample molecule, and wherein the alignment positions of the chromatogram-related output comprise the first and the second alignment positions.

31. The method of claim 24, co-amplifying the spike-in mixture comprises amplifying the synthetic-target associated molecules and the sample molecules with a set of primers, wherein the set of primers include nucleotide sequences that are complementary or reverse complementary to the target matching region of the synthetic target-associated molecules and are complementary or reverse complementary to the first region of the infectious disease's nucleotide sequence.

32. (canceled)

33. (canceled)

34. (canceled)

35. (canceled)

36. The method of claim 30, wherein the primers further comprise one or more fluorescently labeled tags attached at the 5′ end of the primer sequences.

37. (canceled)

38. The method of claim 35, wherein the co-amplified mixture comprises synthetic target-associated amplicon products and, when the infectious disease is present in the sample, infectious disease amplicon products, the synthetic target-associated amplicon products comprising a nucleotide length that is shorter or longer than the nucleotide length of the second region of the infectious disease's nucleotide sequence.

39. (canceled)

40. (canceled)

41. (canceled)

42. The method of claim 25, wherein the peak associated with the second region of the infectious disease's nucleotide sequence includes a peak intensity position that is offset as compared to a peak intensity position of the target-variation region of the synthetic target-associated sample, the offset corresponding to the insertion or deletion of one or more nucleotides in the target-variation region of the synthetic target-associated sample.

43. (canceled)

44. (canceled)

45. (canceled)

46. The method of claim 24, wherein the synthetic target-associated molecule is a DNA or RNA molecule.

47. The method of claim 24, wherein the sample molecule is a DNA or RNA molecule.

48. The method of claim 24, wherein the infectious disease is: coronavirus, influenza virus, rhinovirus, respiratory syncytial virus, metapneumovirus, adenovirus, or boca virus.

49. (canceled)

50. (canceled)

51. A method of detecting the presence or absence of one or more infectious diseases in a sample obtained from a subject, the method comprising:
generating a spike-in mixture including sample molecules from the sample and synthetic target-associated molecules, wherein the synthetic target-associated molecules comprise:
a plurality of target-matching regions, each target matching region matching a corresponding nucleotide sequence in a first region of a corresponding infectious disease's RNA or DNA from a set of infectious diseases, and
a plurality of target-variation regions, each target-variation region is distinguishable from a second region of the corresponding infectious disease's RNA or DNA from the set of infectious diseases, the target-variation region having a nucleotide sequence with an insertion or deletion as compared to a corresponding nucleotide sequence in the second region of the corresponding infectious disease's RNA or DNA from the set of infectious diseases;
co-amplifying the synthetic target-associated molecules and sample molecules to generate a co-amplified spike-in mixture comprising amplicon products, wherein an amplicon product generated by amplifying a given infectious disease's RNA or DNA differs by a predetermined length from an amplicon product generated by amplifying the corresponding target matching and target variation regions of the synthetic target-associated molecules;
performing capillary electrophoresis on the co-amplified spike-in mixture to determine a chromatogram-related output comprising a plurality of chromatogram intensities corresponding to the amplicon products; and
determining the presence or absence of at least one infectious disease based on a chromatogram intensity associated with the amplicon product generated by amplifying the at least one infectious disease's RNA or DNA and a chromatogram intensity associated with an amplicon product having a length that differs by the predetermined length from the amplicon product generated by amplifying the at least one infectious disease's RNA or DNA.

52. The method of claim 50, wherein the synthetic target-associated molecules comprise:
a first target-matching region that matches a corresponding nucleotide sequence in a first region of a first infectious disease's RNA or DNA, and
a first target-variation region that is distinguishable from a second region of the first infectious disease's RNA or DNA, the target-variation region having a nucleotide sequence with an insertion or deletion as compared to a corresponding nucleotide sequence in the second region of the first infectious disease's RNA or DNA;
a second target-matching region that matches a corresponding nucleotide sequence in a first region of a second infectious disease's RNA or DNA, and
a second target-variation region that is distinguishable from a second region of the second infectious disease's RNA or DNA, the target-variation region having a nucleotide sequence with an insertion or deletion as compared to a corresponding nucleotide sequence in the second region of the second infectious disease's RNA or DNA.

53. The method of claim 51, wherein the synthetic target-associated molecules further comprise:
a third target-matching region that matches a corresponding nucleotide sequence in a first region of the third infectious disease's RNA or DNA, and
a third target-variation region that is distinguishable from a second region of the third infectious disease's RNA or DNA, the target-variation region having a nucleotide sequence with an insertion or deletion as compared to a corresponding nucleotide sequence in the second region of the third infectious disease's RNA or DNA.

54. The method of claim 51, wherein amplicon products associated with the first infectious disease have a sample nucleotide length that is different by a second predetermined amount than that of sample amplicon products associated with the second infectious disease.

55. The method of claim 51, wherein sets of primers used in co-amplification comprise a first set of primers including nucleotide sequences that are complementary to the first target matching region of the synthetic target-associated molecules and are complementary to the first region of the first infectious disease's RNA or DNA.

56. (canceled)

57. (canceled)

58. (canceled)

59. (canceled)

60. The method of claim 51, wherein the co-amplified spike-in mixture comprises amplicon products of the synthetic target associated molecules and, when the corresponding infectious disease is present in the sample, amplicon products of the infectious disease's RNA or DNA.

61. The method of claim 59, wherein the synthetic target-associated amplicon products have a shorter or longer nucleotide length as compared to a nucleotide length of the sample amplicon products by 1-100 nucleotides.

62. (canceled)

63. (canceled)

64. (canceled)

65. The method of claim 59, wherein the synthetic target-associated amplicon products comprise a fluorescent label that is distinct in color from a fluorescent label of the amplicon products of the infectious disease's RNA or DNA.

66. The method of claim 51, wherein the synthetic target-associated amplicon products comprise a first set of target-associated amplicon products comprising the first target-matching region and the first target-variation region, and a second set of target-associated amplicon products comprising the second target-matching region and the second target-variation region, wherein the first set of target-associated amplicon products comprise a fluorescent label that is distinct from a fluorescent label of the second set of target-associated amplicon products.

67. The method of claim 65, wherein the amplicon products further comprise a first set of sample amplicon products for detecting a first infectious disease and a second set of sample amplicon products for detecting a second infectious disease, wherein the first set of sample amplicon products comprise a fluorescent label that is distinct from a fluorescent label of the second set of sample amplicon products.

68. The method of claim 66, wherein the first set of sample amplicon products and the first set of target-associated amplicon products comprise the same type of fluorescent label.

69. The method of claim 66, wherein the second set of sample amplicon products and the second set of target-associated amplicon products comprise the same type of fluorescent label.

70. (canceled)

71. (canceled)

72. (canceled)

73. (canceled)

74. (canceled)

75. The method of claim 50, wherein the chromatogram intensities comprise one or more intensity peaks.

76. (canceled)

77. The method of claim 74, wherein the one or more intensity peaks of the synthetic target-associated amplicon products is associated with the synthetic target-associated molecule nucleotide length, and wherein the one or more intensity peaks of the sample amplicon products is associated with the sample nucleotide length.

78. The method of claim 76, further comprising calculating the ratio of intensity peaks of the sample amplicon products to the intensity peaks of the synthetic target-associated amplicon products.

79. The method of claim 77, wherein the intensity peak of the region of the sample amplicon products that corresponds to the target-variation region of the synthetic target-associated amplicon products includes a peak intensity position that is offset as compared to the peak intensity position of the target-variation region, wherein the peak intensity position is offset by one or more nucleotides associated with the insertion or deletion of the target-variation region.

80. The method of claim 78, wherein comparing the chromatogram intensities comprises:
comparing a location of the intensity peak associated with the first target-variation region of the synthetic target-associated amplicon products and a location of the intensity peak of the region of the sample amplicon products of the subject; or
calculating the ratio between the intensity peak associated with the first target-variation region of the synthetic target-associated amplicon products and intensity peak of the region of the sample amplicon products of the subject.

81. (canceled)

82. The method of claim 79, wherein the method further comprises:
aggregating peak intensities across each synthetic target-associated amplicon products of the same nucleotide length;
aggregating peak intensities across each sample amplicon product of the same nucleotide length; and
comparing the aggregated peak intensities of the target-associated amplicon products and the sample amplicon products.

83. The method of claim 81, wherein the method further comprises computing a ratio between the aggregated sample amplicon product peak intensity and the aggregated synthetic target-associated amplicon product peak intensity.

84. (canceled)

85. (canceled)

86. (canceled)

87. (canceled)

88. (canceled)

89. (canceled)

90. A method of detecting the presence or absence of one or more infectious diseases in a sample obtained from a subject, the method comprising:
generating a spike-in mixture including sample molecules from the sample and synthetic target-associated molecules, wherein the synthetic target-associated molecules comprise:
a first target-matching region that matches a corresponding nucleotide sequence in a first region of a first infectious disease's RNA or DNA; and
a target-variation region that is distinguishable from a second region of the first infectious disease's RNA or DNA, the target-variation region having a nucleotide sequence with an insertion or deletion as compared to a corresponding nucleotide sequence in the second region of the first infectious disease's RNA or DNA;
co-amplifying the synthetic target-associated molecules and sample molecules from a subject with a set of primers to generate a co-amplified mixture of synthetic target-associated amplicon products, and sample amplicon products when the infectious disease is present in the sample, wherein co-amplifying the spike-in mixture comprises amplifying the synthetic target-associated molecules and the sample molecules with a set of primer sequences, wherein the set of primer sequences include nucleotide sequences that are complementary or reverse complementary to the first target matching region of the synthetic target-associated molecules and are complementary or reverse complementary to the first region of the first infectious disease's RNA or DNA, wherein the synthetic target-associated amplicon products have a target-associated nucleotide length that is different by a predetermined amount than a sample nucleotide length of the sample amplicon products;
performing capillary electrophoresis on the co-amplified spike-in mixture to determine a chromatogram-related output comprising a plurality of chromatogram intensities, including an intensity associated with:
amplicon products having the target-associated nucleotide length; and
amplicon products having the sample nucleotide length; and
determining the presence or absence of first infectious disease by comparing the chromatogram intensities associated with the amplicon products having the target-associated nucleotide length and amplicon products having the sample nucleotide length.

91. (canceled)

92. (canceled)

93. The method of claim 89, wherein the amplicon products of the synthetic target-associated molecules have a shorter or longer nucleotide length as compared to amplicon products of the sample molecule by 1-100 nucleotides.

94. (canceled)

95. (canceled)

96. The method of claim 89, wherein the set of primers comprise one or more fluorescently labeled tags.

97. (canceled)

98. (canceled)

99. (canceled)

100. The method of claim 89, wherein the chromatogram intensities comprise one or more intensity peaks.

101. (canceled)

102. The method of claim 99, wherein the one or more intensity peaks of the synthetic target-associated amplicon products is associated with a nucleotide length of the synthetic target-associated amplicon products, and wherein the one or more intensity peaks of the sample amplicon products is associated with a nucleotide length of the sample amplicon products.

103. The method of claim 89, wherein the method further comprises calculating the ratio of intensity peaks of the sample amplicon products to the intensity peaks of the synthetic target-associated amplicon products.

104. The method of claim 102, wherein the intensity peak of the region of the sample molecules that corresponds to the target-variation region of the synthetic target-associated molecules includes a peak intensity position that is offset as compared to the peak intensity position of the target-variation region of the synthetic target-associated molecules, wherein the peak intensity position is offset by one or more nucleotides associated with the insertion or deletion of the target-variation region.

105. The method of claim 102, wherein comparing the chromatogram intensities comprises comparing a location of the intensity peak associated with the first target-variation region of the synthetic target-associated amplicon products and a location of the intensity peak of the region of the sample amplicon products of the subject.

106. The method of claim 89, wherein the method further comprises comparing the chromatogram intensities comprises calculating the ratio between the intensity peak associated with the first target-variation region of the synthetic target-associated amplicon products and intensity peak of the region of the sample amplicon products of the subject.

107. The method of claim 89, wherein said comparing further comprises:
aggregating peak intensities across each synthetic target-associated amplicon products of the same nucleotide length;
aggregating peak intensities across each sample amplicon product of the same nucleotide lengths, and
comparing the aggregated peaks intensities.

108. The method of claim 106, wherein the method further comprises computing a ratio between the aggregated sample amplicon product peak intensity and the aggregated synthetic target-associated amplicon product peak intensity.

109. (canceled)

110. (canceled)

111. (canceled)

112. (canceled)

113. (canceled)

114. (canceled)

115. (canceled)

116. (canceled)
